(19) United States
(12) Patent Application Publication
(10) Pub. No.: US 2024/0124864 A1
(43) Pub. Date: Apr. 18, 2024

(54) BLOOD GLUCOSE STABILIZING METHODS AND COMPOSITIONS

(71) Applicant: Digestiva, Inc., Davis, CA (US)

(72) Inventor: Wai Shun Mak, Sacramento, CA (US)

(21) Appl. No.: 18/537,715

(22) Filed: Dec. 12, 2023

Related U.S. Application Data
(63) Continuation of application No. 18/484,305, filed on Oct. 10, 2023.
Application No. 18/484,305 is a continuation of application No. 18/184,476, filed on Mar. 15, 2023, now Pat. No. 11,821,013.
Application No. 18/184,476 is a continuation of application No. PCT/US2023/061253, filed on Jan. 25, 2023.
(60) Provisional application No. 63/303,112, filed on Jan. 26, 2022.

(51) Int. Cl. C12N 9/64; A61K 9/00; A61K 36/48; A61P 3/08

(52) U.S. Cl. CPC C12N 9/6424; A61K 9/0056; A61K 36/48; A61P 3/08

(57) ABSTRACT

Provided and described herein are methods and compositions for decreasing blood glucose, reducing an increase in blood glucose, and/or lowering the glycemic index of a foodstuff.

### CROSS-REFERENCE

This application is a continuation U.S. patent application Ser. No. 18/484,305 filed Oct. 10, 2023, which is a continuation of U.S. patent application Ser. No. 18/184,476, filed Mar. 15, 2023 (now U.S. Pat. No. 11,821,013, issued Nov. 21, 2023), which is a continuation of International Application No. PCT/US2023/061253, filed Jan. 25, 2023, which claims the benefit of U.S. Provisional Application No. 63/303,112 filed Jan. 26, 2022, the entirety of which are hereby incorporated by reference herein.

### BACKGROUND

High blood glucose is well-known to have a plethora of negative health effects. Long-term complications from hyperglycemia can range from cardiovascular disease, neuropathy, damage to kidney and blood vessels, to problems in bone and joint. The most used method to control one's blood glucose level is through controlling dietary sugar intake. While this is a sound and proven strategy, it requires a lot of attention from the individual to understand and calculate the amount of carbohydrate consumed every meal. Medications such as insulin are widely used, however, an array of issues can be associated with effectively controlling blood glucoses levels through medication.

### SEQUENCE LISTING

The instant application contains a Sequence Listing which has been submitted electronically in XML format and is hereby incorporated by reference in its entirety. Said XML copy, created on Dec. 28, 2023, is named 062773-503C03US.xml and is 40,164 bytes bytes in size.

### SUMMARY

Despite a growing understanding of the biology and etiology of diseases associate with or arising because of high levels of blood glucose, a tremendous burden is nonetheless placed on individuals that cannot regulated blood glucoses levels independently of any intervention. For both medicinal treatments (e.g., insulin hormone) and non-medicinal treatments (e.g., dietary monitoring), both may be viewed as substantive interventions having innate barriers to effective treatment and management of high blood glucose-related disease. Such innate barriers include, for example, treatment adherence (e.g., resulting from treatment complexity), financial burden and access (e.g., out of pocket costs for both insured and uninsured individuals), and other patient-, prescription-, and prescriber-related factors. Such barriers and the resulting effects on the treatment of high blood glucose-related diseases, place a substantial health and economic burden upon individuals and healthcare systems.

Provided and described herein are compositions and methods useful for regulating (e.g., decreasing, stabilizing, etc.) high blood glucose. The provided method and compositions are based on, in part, the surprising discovery that the regulation of high blood glucose can be achieved by providing a legume protein (e.g., pea protein) and an S53 family protease to an individual. In some embodiments, the S53 family protease pro-Kumamolisin. In some embodiments, the S53 protease (e.g., pro-Kumamolisin) comprises an amino acid sequence having at least 85% sequence identity to any one of SEQ ID NOs: 1 and 3-11. In some embodiments, the S53 protease (e.g., pro-Kumamolisin) comprises an amino acid sequence having at least 95% sequence identity to any one of SEQ ID NOs: 1 and 3-11. In some embodiments, the S53 protease (e.g., pro-Kumamolisin) comprises an amino acid sequence having at least 98% sequence identity to any one of SEQ ID NOs: 1 and 3-11. In some embodiments, the active site of the S53 protease comprises amino acid residues E266, F295 or A295, S316, W317, G318, A349, A350 or S350, G351, D352, S353 or D353 or A353 or N353, D367 or E367, G462, G463, T464, S465, and A466 of SEQ ID NO:1.

In some embodiments, the S53 protease is active (e.g., as measure by protein digestion) at a pH less than about pH 5. In some embodiments, the S53 protease is active at a pH less than about pH 4.5. In some embodiments, the S53 protease is active at a pH less than about pH 5. In some embodiments, the S53 protease is active at a pH less than about pH 4. In some embodiments, the S53 protease is active at a pH less than about pH 3.5. In some embodiments, the S53 protease is active at a pH less than about pH 3.

In some embodiments, the S53 protease is active at a pH range between about pH 2 and pH 5. In some embodiments, the S53 protease is active at a pH range between about pH 2.5 and pH 4.5. In some embodiments, the S53 protease is at least 50% active (e.g., relative to its max activity) at a pH range between about pH 2.5 and pH 4.5.

In certain instances, the modulation of protein digestion patterns (e.g., by administering a legume protein and an S53 protease (e.g., pro-Kumamolisin)) increases the concentration of certain dietary amino acids in the blood. In such instances, the modulation protein digestion patterns can induce different physiological responses, such as the discovered lowering of blood glucose levels. In certain instances, digesting dietary proteins in the gut e.g., by administering a legume protein and an S53 protease (e.g., pro-Kumamolisin)) produces bioactive peptides that have impactful biological responses, including anti-diabetic effects (e.g., decreasing blood glucose levels). As described and provided herein, a foodstuff comprised of a protein food (e.g., legume protein) with an acid protease (e.g., pro-Kumamolisin) is capable of lowering the blood sugar level (e.g., when consumed with a sugar). Furthermore, in certain instances, the addition of a protein food (e.g., legume protein) with an acid protease (e.g., pro-Kumamolisin) lowers the glycemic index of a foodstuff (e.g., a foodstuff comprising a sugar).

Provided and described herein are methods of decreasing blood glucose in a subject, comprising: administering to the subject a composition comprising: a legume protein; and an S53 protease (e.g., pro-Kumamolisin). Also described and provided are methods of reducing an increase in blood glucose in a subject, comprising: administering to the subject a composition comprising a legume protein; and an S53 protease (e.g., pro-Kumamolisin).

In some embodiments, the S53 protease (e.g., pro-Kumamolisin) comprises an amino acid sequence having at least 85% sequence identity to SEQ ID NO: 1. In some embodiments, the S53 protease (e.g., pro-Kumamolisin) comprises an amino acid sequence having at least 95% sequence identity to SEQ ID NO: 1. In some embodiments, the S53 protease (e.g., pro-Kumamolisin) comprises an amino acid sequence having at least 98% sequence identity to SEQ ID NO: 1.

In some embodiments, wherein the S53 protease (e.g., pro-Kumamolisin) comprises an amino acid sequence of SEQ ID NO: 1. In some embodiments, the S53 protease (e.g., pro-Kumamolisin) comprises an active site comprising amino acid residues E266, F295, S316, W317, G318, A349, A350, G351, D352, S353, D367, G462, G463, T464, S465, and A466 of SEQ ID NO:1. In some embodiments, the S53 protease (e.g., pro-Kumamolisin) comprises an active site comprising one or more amino acid substitutions of residues E266, F295, S316, W317, G318, A349, A350, G351, D352, S353, D367, G462, G463, T464, S465, and A466 of SEQ ID NO:1. In some embodiments, the active site comprises between one and five amino acid substitutions. In some embodiments, the S53 protease (e.g., pro-Kumamolisin) comprises one or more truncations of SEQ ID NO:1, wherein the one or more truncations comprises an N-terminal truncation, a C-terminal truncation, or both an N-terminal and C-terminal truncation.

In some embodiments, the legume protein is a pea protein. In some embodiments, the pea protein is derived from a pea. In certain embodiments, the pea is a garden pea, a sugar pea, a field pea, or any combination thereof. In certain embodiments, the pea is a garden pea. In certain embodiments, the pea is a sugar pea. In certain embodiments, the pea is a field pea. In certain embodiments, the pea is any combination of a garden pea, a sugar pea, and/or a field pea. In some embodiments, the pea is a standard pea, a commoditized pea, a genetically modified pea, or a combination thereof. In certain embodiments, the pea is a smooth pea, a wrinkled pea, or a combination thereof.

In some embodiments, the administering occurs after the subject ingests a foodstuff comprising sugar. In some embodiments, the increase in blood sugar is reduced relative to administering a composition that does not comprise the S53 protease (e.g., pro-Kumamolisin). In some embodiments, the subject self-administers the composition.

In some embodiments, the subject is a mammal. In some embodiments, the mammal is a human. In some embodiments, the human has hyperglycemia. In some embodiments, the human has a condition associated with and/or caused by hyperglycemia. In some embodiments, the condition is cardiovascular disease or neuropathy or diabetic nephropathy or retinopathy or cataract or bone and joint problems or teeth and gum infections.

Also proved are compositions for use in decreasing blood sugar in a subject, the composition comprising a legume protein and an S53 protease (e.g., pro-Kumamolisin). Further provided are compositions for use in reducing an increase blood sugar in a subject, the composition comprising a legume protein and an S53 protease (e.g., pro-Kumamolisin).

In some embodiments, provided is a composition comprising a foodstuff, a legume protein (e.g., pea protein), and an S53 protease (e.g., pro-Kumamolisin) (e.g., any one of the S53 proteases (e.g., pro-Kumamolisin) described herein). In certain embodiments, the composition comprising the foodstuff, the legume protein, and the S53 protease (e.g., pro-Kumamolisin) results in a lower increase in blood sugar after consumption than a second composition comprising the foodstuff alone.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows lowering of blood glucose after administering a legume protein and S53 protease (e.g., pro-Kumamolisin).

FIG. 2 shows lowering of blood glucose after administering a low-carb protein drink and protease.

FIG. 3 shows lowering of blood glucose after administering a protein-fruit smoothie and protease.

FIG. 4 shows proteolytic activity of representative S53 proteases at low pH ranges.

### DETAILED DESCRIPTION

The dysregulation of blood glucose and/or the presence of high blood glucose sugar level is associated with and/or causal of a variety of diseases that affect human health and wellbeing. Such diseases (e.g., diabetes) tend to be complex conditions having burdensome symptoms associated therewith, and are further hallmarked by glucose dysregulation. In certain instances, the methods and compositions provided herein are useful in decreasing and/or reducing blood glucose levels in an individual. Notably, in certain instances, the methods and compositions provided herein are based on the discovery that administering a legume protein (e.g., pea protein) and an S53 protease (e.g., pro-Kumamolisin) lowers (e.g., decreases or reduces) blood sugar levels. Furthermore, in certain instances, the methods and compositions provided herein are useful in inhibiting, reducing, decreasing, and/or preventing an increase in blood glucose levels (e.g., the amount of glucose in the blood).

Legume Proteins

A legume is a plant of the family Fabaceae. A legume, as used herein, generally describes and refers to the fruit or seed of a legume plant. In certain embodiments, the compositions and methods utilized a legume protein. A legume protein, generally described a protein derived from (e.g., obtained from) the fruit or seed of a legume plant. Both intact legume protein and hydrolyzed legume protein sources can be used. In some embodiments, the legume protein is an intact legume protein. In some embodiments, the legume protein is a hydrolyzed legume protein. In some embodiments, the legume protein is provided in solid form. In some embodiments, the legume protein is provided in liquid form. In some embodiments, the legume protein concentrate (e.g., protein material that is obtained from pea upon removal of soluble carbohydrate, ash, and other minor constituents).

In some embodiments, the legume protein is a pea protein. A pea generally refers to and includes the seed or the seed-pod of the pod from a plant belonging to the genus Pisum sativum. A pea protein, generally described a protein derived from (e.g., obtained from) the fruit or seed of a pea plant. Both intact pea protein and hydrolyzed pea protein sources can be used. In some embodiments, the pea protein is an intact legume protein. In some embodiments, the pea protein is a hydrolyzed legume protein. In some embodiments, the pea protein is provided in solid form. In some embodiments, the pea protein is provided in liquid form. In some embodiments, the pea protein concentrate (e.g., protein material that is obtained from pea upon removal of soluble carbohydrate, ash, and other minor constituents). In certain embodiments, the pea is a garden pea, a sugar pea, a field pea, or any combination thereof. In certain embodiments, the pea is a garden pea. In certain embodiments, the pea is a sugar pea. In certain embodiments, the pea is a field pea. In certain embodiments, the pea is any combination of a garden pea, a sugar pea, and/or a field pea. In some embodiments, the pea is a standard pea, a commoditized pea, a genetically modified pea, or a combination thereof. In certain embodiments, the pea is a smooth pea, a wrinkled pea, or a combination thereof.

Proteases

The compositions and methods described herein generally utilize an acid protease. In some embodiments, the acid protease is an S53 family protease. S53 family proteases generally refer to and include the family of serine proteases found in prokaryotes and eukaryotes. In some embodiments, the S53 family proteases refer to and include proteases within and/or identified by MEROPS Accession MER0000995 (e.g., sedolisin, sedolisin-b, tripeptidyl-peptidase I, kumamolisin, kumamolisin-B, physarolisin, aorsin, physarolisin II, kumamolisin-As, grifolisin, scytalidolisin, among others). In some embodiments, the acid protease is an S53 protease (e.g., pro-Kumamolisin). Pro-Kumamolisin generally refers to and includes the thermostable calcium-dependent endopeptidase derived from an acid/thermophilic Bacillus (Bacillus novosp. MN-32). In some embodiments, pro-Kumamolisin refers to and includes NCBI Gene ID: 18765799 (NCBI Reference Sequence XP_007297753.1, XM_007297691.1 to XP_007297753, and/or NW_006763082.1 (137488.139728).

In some embodiments, the S53 protease comprises an amino acid sequence as set forth in SEQ ID NO: 1. In some embodiments, the S53 protease comprises an amino acid sequence having equal to or greater than 80% sequence identity to SEQ ID NO: 1. In some embodiments, the S53 protease comprises an amino acid sequence having equal to or greater than 85% sequence identity to SEQ ID NO: 1. In some embodiments, the S53 protease comprises an amino acid sequence having equal to or greater than 90% sequence identity to SEQ ID NO: 1. In some embodiments, the S53 protease comprises an amino acid sequence having equal to or greater than 95% sequence identity to SEQ ID NO: 1. In some embodiments, the S53 protease comprises an amino acid sequence having equal to or greater than 97% sequence identity to SEQ ID NO: 1. In some embodiments, the S53 protease comprises an amino acid sequence having equal to or greater than 98% sequence identity to SEQ ID NO: 1. In some embodiments, the S53 protease comprises an amino acid sequence having equal to or greater than 99% sequence identity to SEQ ID NO: 1.

In some embodiments, the S53 protease comprises an amino acid sequence as set forth in SEQ ID NO: 2. In some embodiments, the S53 protease comprises an amino acid sequence having equal to or greater than 80% sequence identity to SEQ ID NO: 2. In some embodiments, the S53 protease comprises an amino acid sequence having equal to or greater than 85% sequence identity to SEQ ID NO: 2. In some embodiments, the S53 protease comprises an amino acid sequence having equal to or greater than 90% sequence identity to SEQ ID NO: 2. In some embodiments, the S53 protease comprises an amino acid sequence having equal to or greater than 95% sequence identity to SEQ ID NO: 2. In some embodiments, the S53 protease comprises an amino acid sequence having equal to or greater than 97% sequence identity to SEQ ID NO: 2. In some embodiments, the S53 protease comprises an amino acid sequence having equal to or greater than 98% sequence identity to SEQ ID NO: 2. In some embodiments, the S53 protease comprises an amino acid sequence having equal to or greater than 99% sequence identity to SEQ ID NO: 2.

In some embodiments, the S53 protease comprises an amino acid sequence as set forth in SEQ ID NO: 3. In some embodiments, the S53 protease comprises an amino acid sequence having equal to or greater than 80% sequence identity to SEQ ID NO: 3. In some embodiments, the S53 protease comprises an amino acid sequence having equal to or greater than 85% sequence identity to SEQ ID NO: 3. In some embodiments, the S53 protease comprises an amino acid sequence having equal to or greater than 90% sequence identity to SEQ ID NO: 3. In some embodiments, the S53 protease comprises an amino acid sequence having equal to or greater than 95% sequence identity to SEQ ID NO: 3. In some embodiments, the S53 protease comprises an amino acid sequence having equal to or greater than 97% sequence identity to SEQ ID NO: 3. In some embodiments, the S53 protease comprises an amino acid sequence having equal to or greater than 98% sequence identity to SEQ ID NO: 3. In some embodiments, the S53 protease comprises an amino acid sequence having equal to or greater than 99% sequence identity to SEQ ID NO: 3.

In some embodiments, the S53 protease comprises an amino acid sequence as set forth in SEQ ID NO: 4. In some embodiments, the S53 protease comprises an amino acid sequence having equal to or greater than 80% sequence identity to SEQ ID NO: 4. In some embodiments, the S53 protease comprises an amino acid sequence having equal to or greater than 85% sequence identity to SEQ ID NO: 4. In some embodiments, the S53 protease comprises an amino acid sequence having equal to or greater than 90% sequence identity to SEQ ID NO: 4. In some embodiments, a pro-Kumamolisin comprises an amino acid sequence having equal to or greater than 95% sequence identity to SEQ ID NO: 4. In some embodiments, the S53 protease comprises an amino acid sequence having equal to or greater than 97% sequence identity to SEQ ID NO: 4. In some embodiments, the S53 protease comprises an amino acid sequence having equal to or greater than 98% sequence identity to SEQ ID NO: 4. In some embodiments, the S53 protease comprises an amino acid sequence having equal to or greater than 99% sequence identity to SEQ ID NO: 4.

In some embodiments, the S53 protease comprises an amino acid sequence as set forth in SEQ ID NO: 5. In some embodiments, the S53 protease comprises an amino acid sequence having equal to or greater than 80% sequence identity to SEQ ID NO: 5. In some embodiments, the S53 protease comprises an amino acid sequence having equal to or greater than 85% sequence identity to SEQ ID NO: 5. In some embodiments, the S53 protease comprises an amino acid sequence having equal to or greater than 90% sequence identity to SEQ ID NO: 5. In some embodiments, the S53 protease comprises an amino acid sequence having equal to or greater than 95% sequence identity to SEQ ID NO: 5. In some embodiments, the S53 protease comprises an amino acid sequence having equal to or greater than 97% sequence identity to SEQ ID NO: 5. In some embodiments, the S53 protease comprises an amino acid sequence having equal to or greater than 98% sequence identity to SEQ ID NO: 5. In some embodiments, the S53 protease comprises an amino acid sequence having equal to or greater than 99% sequence identity to SEQ ID NO: 5.

In some embodiments, the S53 protease comprises an amino acid sequence as set forth in SEQ ID NO: 6. In some embodiments, the S53 protease comprises an amino acid sequence having equal to or greater than 80% sequence identity to SEQ ID NO: 6. In some embodiments, the S53 protease comprises an amino acid sequence having equal to or greater than 85% sequence identity to SEQ ID NO: 6. In some embodiments, the S53 protease comprises an amino acid sequence having equal to or greater than 90% sequence identity to SEQ ID NO: 6. In some embodiments, the S53 protease comprises an amino acid sequence having equal to or greater than 95% sequence identity to SEQ ID NO: 6. In some embodiments, the S53 protease comprises an amino acid sequence having equal to or greater than 97% sequence identity to SEQ ID NO: 6. In some embodiments, the S53 protease comprises an amino acid sequence having equal to or greater than 98% sequence identity to SEQ ID NO: 6. In some embodiments, the S53 protease comprises an amino acid sequence having equal to or greater than 99% sequence identity to SEQ ID NO: 6.

In some embodiments, the S53 protease comprises an amino acid sequence as set forth in SEQ ID NO: 7. In some embodiments, the S53 protease comprises an amino acid sequence having equal to or greater than 80% sequence identity to SEQ ID NO: 7. In some embodiments, the S53 protease comprises an amino acid sequence having equal to or greater than 85% sequence identity to SEQ ID NO: 7. In some embodiments, the S53 protease comprises an amino acid sequence having equal to or greater than 90% sequence identity to SEQ ID NO: 7. In some embodiments, the S53 protease comprises an amino acid sequence having equal to or greater than 95% sequence identity to SEQ ID NO: 7. In some embodiments, the S53 protease comprises an amino acid sequence having equal to or greater than 97% sequence identity to SEQ ID NO: 7. In some embodiments, the S53 protease comprises an amino acid sequence having equal to or greater than 98% sequence identity to SEQ ID NO: 7. In some embodiments, the S53 protease comprises an amino acid sequence having equal to or greater than 99% sequence identity to SEQ ID NO: 7.

In some embodiments, the S53 protease comprises an amino acid sequence as set forth in SEQ ID NO: 8. In some embodiments, the S53 protease comprises an amino acid sequence having equal to or greater than 80% sequence identity to SEQ ID NO: 8. In some embodiments, the S53 protease comprises an amino acid sequence having equal to or greater than 85% sequence identity to SEQ ID NO: 8. In some embodiments, the S53 protease comprises an amino acid sequence having equal to or greater than 90% sequence identity to SEQ ID NO: 8. In some embodiments, the S53 protease comprises an amino acid sequence having equal to or greater than 95% sequence identity to SEQ ID NO: 8. In some embodiments, the S53 protease comprises an amino acid sequence having equal to or greater than 97% sequence identity to SEQ ID NO: 8. In some embodiments, the S53 protease comprises an amino acid sequence having equal to or greater than 98% sequence identity to SEQ ID NO: 8. In some embodiments, the S53 protease comprises an amino acid sequence having equal to or greater than 99% sequence identity to SEQ ID NO: 8.

In some embodiments, the S53 protease comprises an amino acid sequence as set forth in SEQ ID NO: 9. In some embodiments, the S53 protease comprises an amino acid sequence having equal to or greater than 80% sequence identity to SEQ ID NO: 9. In some embodiments, the S53 protease comprises an amino acid sequence having equal to or greater than 85% sequence identity to SEQ ID NO: 9. In some embodiments, the S53 protease comprises an amino acid sequence having equal to or greater than 90% sequence identity to SEQ ID NO: 9. In some embodiments, the S53 protease comprises an amino acid sequence having equal to or greater than 95% sequence identity to SEQ ID NO: 9. In some embodiments, the S53 protease comprises an amino acid sequence having equal to or greater than 97% sequence identity to SEQ ID NO: 9. In some embodiments, the S53 protease comprises an amino acid sequence having equal to or greater than 98% sequence identity to SEQ ID NO: 9. In some embodiments, the S53 protease comprises an amino acid sequence having equal to or greater than 99% sequence identity to SEQ ID NO: 9.

In some embodiments, the S53 protease comprises an amino acid sequence as set forth in SEQ ID NO: 10. In some embodiments, the S53 protease comprises an amino acid sequence having equal to or greater than 80% sequence identity to SEQ ID NO: 10. In some embodiments, the S53 protease comprises an amino acid sequence having equal to or greater than 85% sequence identity to SEQ ID NO: 10. In some embodiments, the S53 protease comprises an amino acid sequence having equal to or greater than 90% sequence identity to SEQ ID NO: 10. In some embodiments, the S53 protease comprises an amino acid sequence having equal to or greater than 95% sequence identity to SEQ ID NO: 10. In some embodiments, the S53 protease comprises an amino acid sequence having equal to or greater than 97% sequence identity to SEQ ID NO: 10. In some embodiments, the S53 protease comprises an amino acid sequence having equal to or greater than 98% sequence identity to SEQ ID NO: 10. In some embodiments, the S53 protease comprises an amino acid sequence having equal to or greater than 99% sequence identity to SEQ ID NO: 10.

In some embodiments, the S53 protease comprises an amino acid sequence as set forth in SEQ ID NO: 11. In some embodiments, the S53 protease comprises an amino acid sequence having equal to or greater than 80% sequence identity to SEQ ID NO: 11. In some embodiments, the S53 protease comprises an amino acid sequence having equal to or greater than 85% sequence identity to SEQ ID NO: 11. In some embodiments, the S53 protease comprises an amino acid sequence having equal to or greater than 90% sequence identity to SEQ ID NO: 11. In some embodiments, the S53 protease comprises an amino acid sequence having equal to or greater than 95% sequence identity to SEQ ID NO: 11. In some embodiments, the S53 protease comprises an amino acid sequence having equal to or greater than 97% sequence identity to SEQ ID NO: 11. In some embodiments, the S53 protease comprises an amino acid sequence having equal to or greater than 98% sequence identity to SEQ ID NO: 11. In some embodiments, the S53 protease comprises an amino acid sequence having equal to or greater than 99% sequence identity to SEQ ID NO: 11.

In some embodiments, the active site of the S53 protease comprises amino acid residues E266, F295 or A295, S316, W317, G318, A349, A350 or S350, G351, D352, S353 or D353 or A353 or N353, D367 or E367, G462, G463, T464, S465, and A466 of SEQ ID NO:1.

The determination of percent identity or percent similarity between two sequences can be accomplished using a mathematical algorithm. In some embodiments, a non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. Alternatively, in some embodiments, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. In some embodiments, a non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. In some embodiments, sequence alignment is be carried out using the CLUSTAL algorithm, as described by Higgins et al., 1996, Methods Enzymol. 266:383-402.

In some embodiments, the active site of an S53 protease (e.g., pro-Kumamolisin) comprises amino acid residues E266, F295, S316, W317, G318, A349, A350, G351, D352, S353, D367, G462, G463, T464, S465, and A466 of SEQ ID NO:1. In some embodiments, the S53 protease (e.g., pro-Kumamolisin) comprises an active site comprising one or more amino acid substitutions of residues E266, F295, S316, W317, G318, A349, A350, G351, D352, S353, D367, G462, G463, T464, S465, and A466 of SEQ ID NO:1. In some embodiments, the active site comprises between one and five amino acid substitutions.

In some embodiments, the active site comprises between one or more amino acid substitutions. In some embodiments, the active site comprises between two or more amino acid substitutions. In some embodiments, the active site comprises between three or more amino acid substitutions. In some embodiments, the active site comprises between four or more amino acid substitutions.

In some embodiments, the active site comprises one amino acid substitution. In some embodiments, the active site comprises two amino acid substitutions. In some embodiments, the active site comprises three amino acid substitutions. In some embodiments, the active site comprises four amino acid substitutions. In some embodiments, the active site comprises five amino acid substitutions.

An amino acid generally refers to and/or includes naturally occurring amino acids, unnatural amino acids, amino acid analogues and amino acid mimetics that function in a manner similar to a naturally occurring amino acids. Amino acids are generally referred to herein by either their name, the commonly known three letter symbols, or by the one-letter symbols recommended by the IUPAC-IUB Biochemical Nomenclature Commission. As used herein, naturally occurring amino acids include and/or refer to amino acids which are generally found in nature and are not manipulated by man. In some embodiments, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (Por Pro), glutamine (Q or Gln), arginine (R or Arg), serine (S or Ser), threonine (T or Thr), valine (V or Val), tryptophan (W or Trp), and tyrosine (Y or Tyr).

In some embodiments, a non-polar amino acid can be substituted and replaced with another non-polar amino acid, wherein non-polar amino acids include alanine, leucine, isoleucine, valine, glycine, proline, phenylalanine, tryptophan and methionine. In some embodiments, a neutrally charged polar amino acids can be substituted and replaced with another neutrally charged polar amino acid, wherein neutrally charged polar amino acids include serine, threonine, cysteine, tyrosine, asparagine, and glutamine. In some embodiments, a positively charged amino acid can be substituted and replaced with another positively charged amino acid, wherein positively charged amino acids include arginine, lysine and histidine. In some embodiments, a negatively charged amino acid can be substituted and replaced with another negatively charged amino acid, wherein negatively charged amino acids include aspartic acid and glutamic acid. As used herein, a peptide includes and/or refers to any of various natural or synthetic compounds containing two or more amino acids joined by a peptide bond that link the carboxyl group of one amino acid to the amino group of another. As also used herein, amino acid refers to and/or includes naturally occurring amino acids, unnatural amino acids, amino acid analogues and amino acid mimetics that function in a manner similar to a naturally occurring amino acids. Amino acids are generally referred to herein by either their name, the commonly known three letter symbols, or by the one-letter symbols recommended by the IUPAC-IUB Biochemical Nomenclature Commission.

In some embodiments, the active site comprises between one and five amino acid substitutions. In some embodiments, the S53 protease (e.g., pro-Kumamolisin) comprises one or more truncations of SEQ ID NO:1, wherein the one or more truncations comprises an N-terminal truncation, a C-terminal truncation, or both an N-terminal and C-terminal truncation.

In some embodiments, the S53 protease is active (e.g., as measure by protein digestion) at a pH less than about pH 5. In some embodiments, the S53 protease is active at a pH less than about pH 4.5. In some embodiments, the S53 protease is active at a pH less than about pH 5. In some embodiments, the S53 protease is active at a pH less than about pH 4. In some embodiments, the S53 protease is active at a pH less than about pH 3.5. In some embodiments, the S53 protease is active at a pH less than about pH 3.

In some embodiments, the S53 protease is active at a pH range between about pH 2 and pH 5 In some embodiments, the S53 protease is active at a pH range between about pH 2.5 and pH 4.5. In some embodiments, the S53 protease is at least 50% active (e.g., relative to its max activity) at a pH range between about pH 2.5 and pH 4.5.

The S53 protease (e.g., pro-Kumamolisin) can be administered as part of a composition comprising the S53 protease (e.g., pro-Kumamolisin). The composition can further comprise the legume protein. In some embodiments, provided is a composition comprising the S53 protease (e.g., pro-Kumamolisin). In certain embodiments, the composition further comprises a legume protein (e.g., pea protein).

In some embodiments, provided is a composition comprising a foodstuff, a legume protein (e.g., pea protein), and an S53 protease (e.g., pro-Kumamolisin) (e.g., any one of the S53 proteases (e.g., pro-Kumamolisin) described herein). In certain embodiments, the foodstuff comprises a sugar. In certain embodiments, the sugar comprises sucrose, lactose, maltose, or another di-, tri-, or polysaccharides that contain glucose as a monomer. In certain embodiments, the sugar comprises sucrose. In certain embodiments, the sugar comprises lactose. In certain embodiments, the sugar comprises maltose. In certain embodiments, the sugar comprises di-, tri-, or polysaccharides that contain glucose as a monomer. In certain instances, the addition of the legume protein (e.g., pea protein) and the S53 protease (e.g., pro-Kumamolisin) lowers the glycemic index of the composition comprising the foodstuff. In certain embodiments, the composition comprising the foodstuff, the legume protein, and the S53 protease (e.g., pro-Kumamolisin) results in a lower increase in blood sugar after consumption than a second composition comprising the foodstuff alone.

In some embodiments, composition comprises about 5 grams (g) of legume protein (e.g., pea protein) to about 60 grams (g) of legume protein (e.g., pea protein). In some embodiments, composition comprises about 5 grams (g) of legume protein (e.g., pea protein) to about 10 grams (g) of legume protein (e.g., pea protein), about 5 grams (g) of legume protein (e.g., pea protein) to about 15 grams (g) of legume protein (e.g., pea protein), about 5 grams (g) of legume protein (e.g., pea protein) to about 20 grams (g) of legume protein (e.g., pea protein), about 5 grams (g) of legume protein (e.g., pea protein) to about 25 grams (g) of legume protein (e.g., pea protein), about 5 grams (g) of legume protein (e.g., pea protein) to about 30 grams (g) of legume protein (e.g., pea protein), about 5 grams (g) of legume protein (e.g., pea protein) to about 35 grams (g) of legume protein (e.g., pea protein), about 5 grams (g) of legume protein (e.g., pea protein) to about 40 grams (g) of legume protein (e.g., pea protein), about 5 grams (g) of legume protein (e.g., pea protein) to about 45 grams (g) of legume protein (e.g., pea protein), about 5 grams (g) of legume protein (e.g., pea protein) to about 50 grams (g) of legume protein (e.g., pea protein), about 5 grams (g) of legume protein (e.g., pea protein) to about 60 grams (g) of legume protein (e.g., pea protein), about 10 grams (g) of legume protein (e.g., pea protein) to about 15 grams (g) of legume protein (e.g., pea protein), about 10 grams (g) of legume protein (e.g., pea protein) to about 20 grams (g) of legume protein (e.g., pea protein), about 10 grams (g) of legume protein (e.g., pea protein) to about 25 grams (g) of legume protein (e.g., pea protein), about 10 grams (g) of legume protein (e.g., pea protein) to about 30 grams (g) of legume protein (e.g., pea protein), about 10 grams (g) of legume protein (e.g., pea protein) to about 35 grams (g) of legume protein (e.g., pea protein), about 10 grams (g) of legume protein (e.g., pea protein) to about 40 grams (g) of legume protein (e.g., pea protein), about 10 grams (g) of legume protein (e.g., pea protein) to about 45 grams (g) of legume protein (e.g., pea protein), about 10 grams (g) of legume protein (e.g., pea protein) to about 50 grams (g) of legume protein (e.g., pea protein), about 10 grams (g) of legume protein (e.g., pea protein) to about 60 grams (g) of legume protein (e.g., pea protein), about 15 grams (g) of legume protein (e.g., pea protein) to about 20 grams (g) of legume protein (e.g., pea protein), about 15 grams (g) of legume protein (e.g., pea protein) to about 25 grams (g) of legume protein (e.g., pea protein), about 15 grams (g) of legume protein (e.g., pea protein) to about 30 grams (g) of legume protein (e.g., pea protein), about 15 grams (g) of legume protein (e.g., pea protein) to about 35 grams (g) of legume protein (e.g., pea protein), about 15 grams (g) of legume protein (e.g., pea protein) to about 40 grams (g) of legume protein (e.g., pea protein), about 15 grams (g) of legume protein (e.g., pea protein) to about 45 grams (g) of legume protein (e.g., pea protein), about 15 grams (g) of legume protein (e.g., pea protein) to about 50 grams (g) of legume protein (e.g., pea protein), about 15 grams (g) of legume protein (e.g., pea protein) to about 60 grams (g) of legume protein (e.g., pea protein), about 20 grams (g) of legume protein (e.g., pea protein) to about 25 grams (g) of legume protein (e.g., pea protein), about 20 grams (g) of legume protein (e.g., pea protein) to about 30 grams (g) of legume protein (e.g., pea protein), about 20 grams (g) of legume protein (e.g., pea protein) to about 35 grams (g) of legume protein (e.g., pea protein), about 20 grams (g) of legume protein (e.g., pea protein) to about 40 grams (g) of legume protein (e.g., pea protein), about 20 grams (g) of legume protein (e.g., pea protein) to about 45 grams (g) of legume protein (e.g., pea protein), about 20 grams (g) of legume protein (e.g., pea protein) to about 50 grams (g) of legume protein (e.g., pea protein), about 20 grams (g) of legume protein (e.g., pea protein) to about 60 grams (g) of legume protein (e.g., pea protein), about 25 grams (g) of legume protein (e.g., pea protein) to about 30 grams (g) of legume protein (e.g., pea protein), about 25 grams (g) of legume protein (e.g., pea protein) to about 35 grams (g) of legume protein (e.g., pea protein), about 25 grams (g) of legume protein (e.g., pea protein) to about 40 grams (g) of legume protein (e.g., pea protein), about 25 grams (g) of legume protein (e.g., pea protein) to about 45 grams (g) of legume protein (e.g., pea protein), about 25 grams (g) of legume protein (e.g., pea protein) to about 50 grams (g) of legume protein (e.g., pea protein), about 25 grams (g) of legume protein (e.g., pea protein) to about 60 grams (g) of legume protein (e.g., pea protein), about 30 grams (g) of legume protein (e.g., pea protein) to about 35 grams (g) of legume protein (e.g., pea protein), about 30 grams (g) of legume protein (e.g., pea protein) to about 40 grams (g) of legume protein (e.g., pea protein), about 30 grams (g) of legume protein (e.g., pea protein) to about 45 grams (g) of legume protein (e.g., pea protein), about 30 grams (g) of legume protein (e.g., pea protein) to about 50 grams (g) of legume protein (e.g., pea protein), about 30 grams (g) of legume protein (e.g., pea protein) to about 60 grams (g) of legume protein (e.g., pea protein), about 35 grams (g) of legume protein (e.g., pea protein) to about 40 grams (g) of legume protein (e.g., pea protein), about 35 grams (g) of legume protein (e.g., pea protein) to about 45 grams (g) of legume protein (e.g., pea protein), about 35 grams (g) of legume protein (e.g., pea protein) to about 50 grams (g) of legume protein (e.g., pea protein), about 35 grams (g) of legume protein (e.g., pea protein) to about 60 grams (g) of legume protein (e.g., pea protein), about 40 grams (g) of legume protein (e.g., pea protein) to about 45 grams (g) of legume protein (e.g., pea protein), about 40 grams (g) of legume protein (e.g., pea protein) to about 50 grams (g) of legume protein (e.g., pea protein), about 40 grams (g) of legume protein (e.g., pea protein) to about 60 grams (g) of legume protein (e.g., pea protein), about 45 grams (g) of legume protein (e.g., pea protein) to about 50 grams (g) of legume protein (e.g., pea protein), about 45 grams (g) of legume protein (e.g., pea protein) to about 60 grams (g) of legume protein (e.g., pea protein), or about 50 grams (g) of legume protein (e.g., pea protein) to about 60 grams (g) of legume protein (e.g., pea protein). In some embodiments, composition comprises about 5 grams (g) of legume protein (e.g., pea protein), about 10 grams (g) of legume protein (e.g., pea protein), about 15 grams (g) of legume protein (e.g., pea protein), about 20 grams (g) of legume protein (e.g., pea protein), about 25 grams (g) of legume protein (e.g., pea protein), about 30 grams (g) of legume protein (e.g., pea protein), about 35 grams (g) of legume protein (e.g., pea protein), about 40 grams (g) of legume protein (e.g., pea protein), about 45 grams (g) of legume protein (e.g., pea protein), about 50 grams (g) of legume protein (e.g., pea protein), or about 60 grams (g) of legume protein (e.g., pea protein). In some embodiments, composition comprises at least about 5 grams (g) of legume protein (e.g., pea protein), about 10 grams (g) of legume protein (e.g., pea protein), about 15 grams (g) of legume protein (e.g., pea protein), about 20 grams (g) of legume protein (e.g., pea protein), about 25 grams (g) of legume protein (e.g., pea protein), about 30 grams (g) of legume protein (e.g., pea protein), about 35 grams (g) of legume protein (e.g., pea protein), about 40 grams (g) of legume protein (e.g., pea protein), about 45 grams (g) of legume protein (e.g., pea protein), or about 50 grams (g) of legume protein (e.g., pea protein). In some embodiments, composition comprises at most about 10 grams (g) of legume protein (e.g., pea protein), about 15 grams (g) of legume protein (e.g., pea protein), about 20 grams (g) of legume protein (e.g., pea protein), about 25 grams (g) of legume protein (e.g., pea protein), about 30 grams (g) of legume protein (e.g., pea protein), about 35 grams (g) of legume protein (e.g., pea protein), about 40 grams (g) of legume protein (e.g., pea protein), about 45 grams (g) of legume protein (e.g., pea protein), about 50 grams (g) of legume protein (e.g., pea protein), or about 60 grams (g) of legume protein (e.g., pea protein).

In some embodiments, composition comprises about 50 milligrams (mg) of pro-Kumamolisin to about 2,000 milligrams (mg) of pro-Kumamolisin. In some embodiments, composition comprises about 50 milligrams (mg) of pro-Kumamolisin to about 100 milligrams (mg) of pro-Kumamolisin, about 50 milligrams (mg) of pro-Kumamolisin to about 200 milligrams (mg) of pro-Kumamolisin, about 50 milligrams (mg) of pro-Kumamolisin to about 300 milligrams (mg) of pro-Kumamolisin, about 50 milligrams (mg) of pro-Kumamolisin to about 400 milligrams (mg) of pro-Kumamolisin, about 50 milligrams (mg) of pro-Kumamolisin to about 500 milligrams (mg) of pro-Kumamolisin, about 50 milligrams (mg) of pro-Kumamolisin to about 750 milligrams (mg) of pro-Kumamolisin, about 50 milligrams (mg) of pro-Kumamolisin to about 1,000 milligrams (mg) of pro-Kumamolisin, about 50 milligrams (mg) of pro-Kumamolisin to about 1,250 milligrams (mg) of pro-Kumamolisin, about 50 milligrams (mg) of pro-Kumamolisin to about 1,500 milligrams (mg) of pro-Kumamolisin, about 50 milligrams (mg) of pro-Kumamolisin to about 2,000 milligrams (mg) of pro-Kumamolisin, about 100 milligrams (mg) of pro-Kumamolisin to about 200 milligrams (mg) of pro-Kumamolisin, about 100 milligrams (mg) of pro-Kumamolisin to about 300 milligrams (mg) of pro-Kumamolisin, about 100 milligrams (mg) of pro-Kumamolisin to about 400 milligrams (mg) of pro-Kumamolisin, about 100 milligrams (mg) of pro-Kumamolisin to about 500 milligrams (mg) of pro-Kumamolisin, about 100 milligrams (mg) of pro-Kumamolisin to about 750 milligrams (mg) of pro-Kumamolisin, about 100 milligrams (mg) of pro-Kumamolisin to about 1,000 milligrams (mg) of pro-Kumamolisin, about 100 milligrams (mg) of pro-Kumamolisin to about 1,250 milligrams (mg) of pro-Kumamolisin, about 100 milligrams (mg) of pro-Kumamolisin to about 1,500 milligrams (mg) of pro-Kumamolisin, about 100 milligrams (mg) of pro-Kumamolisin to about 2,000 milligrams (mg) of pro-Kumamolisin, about 200 milligrams (mg) of pro-Kumamolisin to about 300 milligrams (mg) of pro-Kumamolisin, about 200 milligrams (mg) of pro-Kumamolisin to about 400 milligrams (mg) of pro-Kumamolisin, about 200 milligrams (mg) of pro-Kumamolisin to about 500 milligrams (mg) of pro-Kumamolisin, about 200 milligrams (mg) of pro-Kumamolisin to about 750 milligrams (mg) of pro-Kumamolisin, about 200 milligrams (mg) of pro-Kumamolisin to about 1,000 milligrams (mg) of pro-Kumamolisin, about 200 milligrams (mg) of pro-Kumamolisin to about 1,250 milligrams (mg) of pro-Kumamolisin, about 200 milligrams (mg) of pro-Kumamolisin to about 1,500 milligrams (mg) of pro-Kumamolisin, about 200 milligrams (mg) of pro-Kumamolisin to about 2,000 milligrams (mg) of pro-Kumamolisin, about 300 milligrams (mg) of pro-Kumamolisin to about 400 milligrams (mg) of pro-Kumamolisin, about 300 milligrams (mg) of pro-Kumamolisin to about 500 milligrams (mg) of pro-Kumamolisin, about 300 milligrams (mg) of pro-Kumamolisin to about 750 milligrams (mg) of pro-Kumamolisin, about 300 milligrams (mg) of pro-Kumamolisin to about 1,000 milligrams (mg) of pro-Kumamolisin, about 300 milligrams (mg) of pro-Kumamolisin to about 1,250 milligrams (mg) of pro-Kumamolisin, about 300 milligrams (mg) of pro-Kumamolisin to about 1,500 milligrams (mg) of pro-Kumamolisin, about 300 milligrams (mg) of pro-Kumamolisin to about 2,000 milligrams (mg) of pro-Kumamolisin, about 400 milligrams (mg) of pro-Kumamolisin to about 500 milligrams (mg) of pro-Kumamolisin, about 400 milligrams (mg) of pro-Kumamolisin to about 750 milligrams (mg) of pro-Kumamolisin, about 400 milligrams (mg) of pro-Kumamolisin to about 1,000 milligrams (mg) of pro-Kumamolisin, about 400 milligrams (mg) of pro-Kumamolisin to about 1,250 milligrams (mg) of pro-Kumamolisin, about 400 milligrams (mg) of pro-Kumamolisin to about 1,500 milligrams (mg) of pro-Kumamolisin, about 400 milligrams (mg) of pro-Kumamolisin to about 2,000 milligrams (mg) of pro-Kumamolisin, about 500 milligrams (mg) of pro-Kumamolisin to about 750 milligrams (mg) of pro-Kumamolisin, about 500 milligrams (mg) of pro-Kumamolisin to about 1,000 milligrams (mg) of pro-Kumamolisin, about 500 milligrams (mg) of pro-Kumamolisin to about 1,250 milligrams (mg) of pro-Kumamolisin, about 500 milligrams (mg) of pro-Kumamolisin to about 1,500 milligrams (mg) of pro-Kumamolisin, about 500 milligrams (mg) of pro-Kumamolisin to about 2,000 milligrams (mg) of pro-Kumamolisin, about 750 milligrams (mg) of pro-Kumamolisin to about 1,000 milligrams (mg) of pro-Kumamolisin, about 750 milligrams (mg) of pro-Kumamolisin to about 1,250 milligrams (mg) of pro-Kumamolisin, about 750 milligrams (mg) of pro-Kumamolisin to about 1,500 milligrams (mg) of pro-Kumamolisin, about 750 milligrams (mg) of pro-Kumamolisin to about 2,000 milligrams (mg) of pro-Kumamolisin, about 1,000 milligrams (mg) of pro-Kumamolisin to about 1,250 milligrams (mg) of pro-Kumamolisin, about 1,000 milligrams (mg) of pro-Kumamolisin to about 1,500 milligrams (mg) of pro-Kumamolisin, about 1,000 milligrams (mg) of pro-Kumamolisin to about 2,000 milligrams (mg) of pro-Kumamolisin, about 1,250 milligrams (mg) of pro-Kumamolisin to about 1,500 milligrams (mg) of pro-Kumamolisin, about 1,250 milligrams (mg) of pro-Kumamolisin to about 2,000 milligrams (mg) of pro-Kumamolisin, or about 1,500 milligrams (mg) of pro-Kumamolisin to about 2,000 milligrams (mg) of pro-Kumamolisin. In some embodiments, composition comprises about 50 milligrams (mg) of pro-Kumamolisin, about 100 milligrams (mg) of pro-Kumamolisin, about 200 milligrams (mg) of pro-Kumamolisin, about 300 milligrams (mg) of pro-Kumamolisin, about 400 milligrams (mg) of pro-Kumamolisin, about 500 milligrams (mg) of pro-Kumamolisin, about 750 milligrams (mg) of pro-Kumamolisin, about 1,000 milligrams (mg) of pro-Kumamolisin, about 1,250 milligrams (mg) of pro-Kumamolisin, about 1,500 milligrams (mg) of pro-Kumamolisin, or about 2,000 milligrams (mg) of pro-Kumamolisin. In some embodiments, composition comprises at least about 50 milligrams (mg) of pro-Kumamolisin, about 100 milligrams (mg) of pro-Kumamolisin, about 200 milligrams (mg) of pro-Kumamolisin, about 300 milligrams (mg) of pro-Kumamolisin, about 400 milligrams (mg) of pro-Kumamolisin, about 500 milligrams (mg) of pro-Kumamolisin, about 750 milligrams (mg) of pro-Kumamolisin, about 1,000 milligrams (mg) of pro-Kumamolisin, about 1,250 milligrams (mg) of pro-Kumamolisin, or about 1,500 milligrams (mg) of pro-Kumamolisin. In some embodiments, composition comprises at most about 100 milligrams (mg) of pro-Kumamolisin, about 200 milligrams (mg) of pro-Kumamolisin, about 300 milligrams (mg) of pro-Kumamolisin, about 400 milligrams (mg) of pro-Kumamolisin, about 500 milligrams (mg) of pro-Kumamolisin, about 750 milligrams (mg) of pro-Kumamolisin, about 1,000 milligrams (mg) of pro-Kumamolisin, about 1,250 milligrams (mg) of pro-Kumamolisin, about 1,500 milligrams (mg) of pro-Kumamolisin, or about 2,000 milligrams (mg) of pro-Kumamolisin.

Methods

As described herein, in certain instances, providing a legume protein (e.g., pea protein) and an S53 protease (e.g., a pro-Kumamolisin) protease lowers (e.g., decreases or reduces) blood sugar levels. Accordingly, provided herein are methods of decreasing blood glucose in a subject, comprising: administering to the subject a composition comprising: a legume protein and an S53 protease (e.g., pro-Kumamolisin). (e.g., wherein the legume protein and the S53 protease (e.g., pro-Kumamolisin) is administered to the subject). Further provided is a legume protein and an S53 protease (e.g., pro-Kumamolisin) for use in a method of decreasing blood glucose in a subject (e.g., wherein the legume protein and the S53 protease (e.g., pro-Kumamolisin) is administered to the subject).

Also provided are methods of reducing an increase in blood glucose in a subject, comprising: administering to the subject a composition comprising a legume protein and an S53 protease (e.g., pro-Kumamolisin). Further provided is the use of a legume protein and an S53 protease (e.g., pro-Kumamolisin) in a method of reducing an increase in blood glucose in a subject(e.g., wherein the legume protein and the S53 protease (e.g., pro-Kumamolisin) is administered to the subject). Furthermore, also provided is a legume protein and an S53 protease (e.g., pro-Kumamolisin) for use in a method of reducing an increase in blood glucose in a subject (e.g., wherein the legume protein and the S53 protease (e.g., pro-Kumamolisin) is administered to the subject).

In certain instances, providing a legume protein (e.g., pea protein) and an S53 protease (e.g., pro-Kumamolisin) stabilizes (e.g., reducing or preventing the extent or degree of change) blood sugar levels associated with the intake of a foodstuff. In some embodiments, provided herein are methods of reducing a change (positive or negative) in blood glucose in a subject, comprising: administering to the subject a composition comprising: a legume protein and an S53 protease (e.g., pro-Kumamolisin) (e.g., wherein the legume protein and the S53 protease (e.g., pro-Kumamolisin) is administered to the subject).

In certain embodiments, administering the legume protein and the S53 protease (e.g., pro-Kumamolisin) reduces the degree or amount of an increase in blood glucose upon intake of a foodstuff. In certain instances, reducing the degree or amount of increase in blood glucose upon intake of a foodstuff can be compared to an increase in blood glucose upon intake of a foodstuff prior to or without administering the legume protein and the S53 protease (e.g., pro-Kumamolisin). In certain embodiments, administering the legume protein and an S53 protease (e.g., pro-Kumamolisin) reduces the degree or amount of decrease in blood glucose upon intake of a foodstuff. In certain instances, reducing the degree or amount of decrease in blood glucose upon intake of a foodstuff can be compared to a decrease in blood glucose upon intake of a foodstuff prior to or without administering the legume protein and the S53 protease (e.g., pro-Kumamolisin). In some embodiments, administering the legume protein and the S53 protease (e.g., pro-Kumamolisin) reduces a change (e.g., increase or decrease) in blood glucose upon intake of a foodstuff. In certain embodiments, reducing a change (e.g., increase or decrease) in blood glucose upon intake of a foodstuff when the legume protein and the S53 protease (e.g., pro-Kumamolisin) is administered is be compared to change in blood glucose upon intake of a foodstuff prior to or without administering the legume protein and/or the S53 protease (e.g., pro-Kumamolisin).

In some embodiments, the S53 protease comprises an amino acid sequence as set forth in SEQ ID NO: 1. In some embodiments, the S53 protease comprises an amino acid sequence having equal to or greater than 80% sequence identity to SEQ ID NO: 1. In some embodiments, the S53 protease comprises an amino acid sequence having equal to or greater than 85% sequence identity to SEQ ID NO: 1. In some embodiments, the S53 protease comprises an amino acid sequence having equal to or greater than 90% sequence identity to SEQ ID NO: 1. In some embodiments, the S53 protease comprises an amino acid sequence having equal to or greater than 95% sequence identity to SEQ ID NO: 1. In some embodiments, the S53 protease comprises an amino acid sequence having equal to or greater than 97% sequence identity to SEQ ID NO: 1. In some embodiments, the S53 protease comprises an amino acid sequence having equal to or greater than 98% sequence identity to SEQ ID NO: 1. In some embodiments, the S53 protease comprises an amino acid sequence having equal to or greater than 99% sequence identity to SEQ ID NO: 1.

In some embodiments, the S53 protease comprises an amino acid sequence as set forth in SEQ ID NO: 2. In some embodiments, the S53 protease comprises an amino acid sequence having equal to or greater than 80% sequence identity to SEQ ID NO: 2. In some embodiments, the S53 protease comprises an amino acid sequence having equal to or greater than 85% sequence identity to SEQ ID NO: 2. In some embodiments, the S53 protease comprises an amino acid sequence having equal to or greater than 90% sequence identity to SEQ ID NO: 2. In some embodiments, the S53 protease comprises an amino acid sequence having equal to or greater than 95% sequence identity to SEQ ID NO: 2. In some embodiments, the S53 protease comprises an amino acid sequence having equal to or greater than 97% sequence identity to SEQ ID NO: 2. In some embodiments, the S53 protease comprises an amino acid sequence having equal to or greater than 98% sequence identity to SEQ ID NO: 2. In some embodiments, the S53 protease comprises an amino acid sequence having equal to or greater than 99% sequence identity to SEQ ID NO: 2.

In some embodiments, the S53 protease comprises an amino acid sequence as set forth in SEQ ID NO: 3. In some embodiments, the S53 protease comprises an amino acid sequence having equal to or greater than 80% sequence identity to SEQ ID NO: 3. In some embodiments, the S53 protease comprises an amino acid sequence having equal to or greater than 85% sequence identity to SEQ ID NO: 3. In some embodiments, the S53 protease comprises an amino acid sequence having equal to or greater than 90% sequence identity to SEQ ID NO: 3. In some embodiments, the S53 protease comprises an amino acid sequence having equal to or greater than 95% sequence identity to SEQ ID NO: 3. In some embodiments, the S53 protease comprises an amino acid sequence having equal to or greater than 97% sequence identity to SEQ ID NO: 3. In some embodiments, the S53 protease comprises an amino acid sequence having equal to or greater than 98% sequence identity to SEQ ID NO: 3. In some embodiments, the S53 protease comprises an amino acid sequence having equal to or greater than 99% sequence identity to SEQ ID NO: 3.

In some embodiments, the S53 protease comprises an amino acid sequence as set forth in SEQ ID NO: 4. In some embodiments, the S53 protease comprises an amino acid sequence having equal to or greater than 80% sequence identity to SEQ ID NO: 4. In some embodiments, the S53 protease comprises an amino acid sequence having equal to or greater than 85% sequence identity to SEQ ID NO: 4. In some embodiments, the S53 protease comprises an amino acid sequence having equal to or greater than 90% sequence identity to SEQ ID NO: 4. In some embodiments, a pro-Kumamolisin comprises an amino acid sequence having equal to or greater than 95% sequence identity to SEQ ID NO: 4. In some embodiments, the S53 protease comprises an amino acid sequence having equal to or greater than 97% sequence identity to SEQ ID NO: 4. In some embodiments, the S53 protease comprises an amino acid sequence having equal to or greater than 98% sequence identity to SEQ ID NO: 4. In some embodiments, the S53 protease comprises an amino acid sequence having equal to or greater than 99% sequence identity to SEQ ID NO: 4.

In some embodiments, the S53 protease comprises an amino acid sequence as set forth in SEQ ID NO: 5. In some embodiments, the S53 protease comprises an amino acid sequence having equal to or greater than 80% sequence identity to SEQ ID NO: 5. In some embodiments, the S53 protease comprises an amino acid sequence having equal to or greater than 85% sequence identity to SEQ ID NO: 5. In some embodiments, the S53 protease comprises an amino acid sequence having equal to or greater than 90% sequence identity to SEQ ID NO: 5. In some embodiments, the S53 protease comprises an amino acid sequence having equal to or greater than 95% sequence identity to SEQ ID NO: 5. In some embodiments, the S53 protease comprises an amino acid sequence having equal to or greater than 97% sequence identity to SEQ ID NO: 5. In some embodiments, the S53 protease comprises an amino acid sequence having equal to or greater than 98% sequence identity to SEQ ID NO: 5. In some embodiments, the S53 protease comprises an amino acid sequence having equal to or greater than 99% sequence identity to SEQ ID NO: 5.

In some embodiments, the S53 protease comprises an amino acid sequence as set forth in SEQ ID NO: 6. In some embodiments, the S53 protease comprises an amino acid sequence having equal to or greater than 80% sequence identity to SEQ ID NO: 6. In some embodiments, the S53 protease comprises an amino acid sequence having equal to or greater than 85% sequence identity to SEQ ID NO: 6. In some embodiments, the S53 protease comprises an amino acid sequence having equal to or greater than 90% sequence identity to SEQ ID NO: 6. In some embodiments, the S53 protease comprises an amino acid sequence having equal to or greater than 95% sequence identity to SEQ ID NO: 6. In some embodiments, the S53 protease comprises an amino acid sequence having equal to or greater than 97% sequence identity to SEQ ID NO: 6. In some embodiments, the S53 protease comprises an amino acid sequence having equal to or greater than 98% sequence identity to SEQ ID NO: 6. In some embodiments, the S53 protease comprises an amino acid sequence having equal to or greater than 99% sequence identity to SEQ ID NO: 6.

In some embodiments, the S53 protease comprises an amino acid sequence as set forth in SEQ ID NO: 7. In some embodiments, the S53 protease comprises an amino acid sequence having equal to or greater than 80% sequence identity to SEQ ID NO: 7. In some embodiments, the S53 protease comprises an amino acid sequence having equal to or greater than 85% sequence identity to SEQ ID NO: 7. In some embodiments, the S53 protease comprises an amino acid sequence having equal to or greater than 90% sequence identity to SEQ ID NO: 7. In some embodiments, the S53 protease comprises an amino acid sequence having equal to or greater than 95% sequence identity to SEQ ID NO: 7. In some embodiments, the S53 protease comprises an amino acid sequence having equal to or greater than 97% sequence identity to SEQ ID NO: 7. In some embodiments, the S53 protease comprises an amino acid sequence having equal to or greater than 98% sequence identity to SEQ ID NO: 7. In some embodiments, the S53 protease comprises an amino acid sequence having equal to or greater than 99% sequence identity to SEQ ID NO: 7.

In some embodiments, the S53 protease comprises an amino acid sequence as set forth in SEQ ID NO: 8. In some embodiments, the S53 protease comprises an amino acid sequence having equal to or greater than 80% sequence identity to SEQ ID NO: 8. In some embodiments, the S53 protease comprises an amino acid sequence having equal to or greater than 85% sequence identity to SEQ ID NO: 8. In some embodiments, the S53 protease comprises an amino acid sequence having equal to or greater than 90% sequence identity to SEQ ID NO: 8. In some embodiments, the S53 protease comprises an amino acid sequence having equal to or greater than 95% sequence identity to SEQ ID NO: 8. In some embodiments, the S53 protease comprises an amino acid sequence having equal to or greater than 97% sequence identity to SEQ ID NO: 8. In some embodiments, the S53 protease comprises an amino acid sequence having equal to or greater than 98% sequence identity to SEQ ID NO: 8. In some embodiments, the S53 protease comprises an amino acid sequence having equal to or greater than 99% sequence identity to SEQ ID NO: 8.

In some embodiments, the S53 protease comprises an amino acid sequence as set forth in SEQ ID NO: 9. In some embodiments, the S53 protease comprises an amino acid sequence having equal to or greater than 80% sequence identity to SEQ ID NO: 9. In some embodiments, the S53 protease comprises an amino acid sequence having equal to or greater than 85% sequence identity to SEQ ID NO: 9. In some embodiments, the S53 protease comprises an amino acid sequence having equal to or greater than 90% sequence identity to SEQ ID NO: 9. In some embodiments, the S53 protease comprises an amino acid sequence having equal to or greater than 95% sequence identity to SEQ ID NO: 9. In some embodiments, the S53 protease comprises an amino acid sequence having equal to or greater than 97% sequence identity to SEQ ID NO: 9. In some embodiments, the S53 protease comprises an amino acid sequence having equal to or greater than 98% sequence identity to SEQ ID NO: 9. In some embodiments, the S53 protease comprises an amino acid sequence having equal to or greater than 99% sequence identity to SEQ ID NO: 9.

In some embodiments, the S53 protease comprises an amino acid sequence as set forth in SEQ ID NO: 10. In some embodiments, the S53 protease comprises an amino acid sequence having equal to or greater than 80% sequence identity to SEQ ID NO: 10. In some embodiments, the S53 protease comprises an amino acid sequence having equal to or greater than 85% sequence identity to SEQ ID NO: 10. In some embodiments, the S53 protease comprises an amino acid sequence having equal to or greater than 90% sequence identity to SEQ ID NO: 10. In some embodiments, the S53 protease comprises an amino acid sequence having equal to or greater than 95% sequence identity to SEQ ID NO: 10. In some embodiments, the S53 protease comprises an amino acid sequence having equal to or greater than 97% sequence identity to SEQ ID NO: 10. In some embodiments, the S53 protease comprises an amino acid sequence having equal to or greater than 98% sequence identity to SEQ ID NO: 10. In some embodiments, the S53 protease comprises an amino acid sequence having equal to or greater than 99% sequence identity to SEQ ID NO: 10.

In some embodiments, the S53 protease comprises an amino acid sequence as set forth in SEQ ID NO: 11. In some embodiments, the S53 protease comprises an amino acid sequence having equal to or greater than 80% sequence identity to SEQ ID NO: 11. In some embodiments, the S53 protease comprises an amino acid sequence having equal to or greater than 85% sequence identity to SEQ ID NO: 11. In some embodiments, the S53 protease comprises an amino acid sequence having equal to or greater than 90% sequence identity to SEQ ID NO: 11. In some embodiments, the S53 protease comprises an amino acid sequence having equal to or greater than 95% sequence identity to SEQ ID NO: 11. In some embodiments, the S53 protease comprises an amino acid sequence having equal to or greater than 97% sequence identity to SEQ ID NO: 11. In some embodiments, the S53 protease comprises an amino acid sequence having equal to or greater than 98% sequence identity to SEQ ID NO: 11. In some embodiments, the S53 protease comprises an amino acid sequence having equal to or greater than 99% sequence identity to SEQ ID NO: 11.

In some embodiments, the active site of the S53 protease comprises amino acid residues E266, F295 or A295, S316, W317, G318, A349, A350 or S350, G351, D352, S353 or D353 or A353 or N353, D367 or E367, G462, G463, T464, S465, and A466 of SEQ ID NO:1.

Any of the S53 proteases (e.g., pro-Kumamolisin) described herein can be used in the methods provided. In some embodiments, the S53 protease (e.g., pro-Kumamolisin) comprises an amino acid sequence having at least 85% sequence identity to SEQ ID NO: 1. In some embodiments, the S53 protease (e.g., pro-Kumamolisin) comprises an amino acid sequence having at least 95% sequence identity to SEQ ID NO: 1. In some embodiments, the S53 protease (e.g., pro-Kumamolisin) comprises an amino acid sequence having at least 98% sequence identity to SEQ ID NO: 1.

In some embodiments, the S53 protease (e.g., pro-Kumamolisin) comprises an amino acid sequence of SEQ ID NO: 1. In some embodiments, the S53 protease (e.g., pro-Kumamolisin) comprises an active site comprising amino acid residues E266, F295, S316, W317, G318, A349, A350, G351, D352, S353, D367, G462, G463, T464, S465, and A466 of SEQ ID NO:1. In some embodiments, the S53 protease (e.g., pro-Kumamolisin) comprises an active site comprising one or more amino acid substitutions of residues E266, F295, S316, W317, G318, A349, A350, G351, D352, S353, D367, G462, G463, T464, S465, and A466 of SEQ ID NO:1. In some embodiments, the active site comprises between one and five amino acid substitutions. In some embodiments, the S53 protease (e.g., pro-Kumamolisin) comprises one or more truncations of SEQ ID NO:1, wherein the one or more truncations comprises an N-terminal truncation, a C-terminal truncation, or both an N-terminal and C-terminal truncation.

In some embodiments, the amount of S53 protease (e.g., pro-Kumamolisin) administered comprises about 50 milligrams (mg) to about 1,500 milligrams (mg). In some embodiments, the amount of S53 protease (e.g., pro-Kumamolisin) administered comprises about 50 milligrams (mg) to about 100 milligrams (mg), about 50 milligrams (mg) to about 150 milligrams (mg), about 50 milligrams (mg) to about 200 milligrams (mg), about 50 milligrams (mg) to about 300 milligrams (mg), about 50 milligrams (mg) to about 400 milligrams (mg), about 50 milligrams (mg) to about 500 milligrams (mg), about 50 milligrams (mg) to about 750 milligrams (mg), about 50 milligrams (mg) to about 1,000 milligrams (mg), about 50 milligrams (mg) to about 1,500 milligrams (mg), about 100 milligrams (mg) to about 150 milligrams (mg), about 100 milligrams (mg) to about 200 milligrams (mg), about 100 milligrams (mg) to about 300 milligrams (mg), about 100 milligrams (mg) to about 400 milligrams (mg), about 100 milligrams (mg) to about 500 milligrams (mg), about 100 milligrams (mg) to about 750 milligrams (mg), about 100 milligrams (mg) to about 1,000 milligrams (mg), about 100 milligrams (mg) to about 1,500 milligrams (mg), about 150 milligrams (mg) to about 200 milligrams (mg), about 150 milligrams (mg) to about 300 milligrams (mg), about 150 milligrams (mg) to about 400 milligrams (mg), about 150 milligrams (mg) to about 500 milligrams (mg), about 150 milligrams (mg) to about 750 milligrams (mg), about 150 milligrams (mg) to about 1,000 milligrams (mg), about 150 milligrams (mg) to about 1,500 milligrams (mg), about 200 milligrams (mg) to about 300 milligrams (mg), about 200 milligrams (mg) to about 400 milligrams (mg), about 200 milligrams (mg) to about 500 milligrams (mg), about 200 milligrams (mg) to about 750 milligrams (mg), about 200 milligrams (mg) to about 1,000 milligrams (mg), about 200 milligrams (mg) to about 1,500 milligrams (mg), about 300 milligrams (mg) to about 400 milligrams (mg), about 300 milligrams (mg) to about 500 milligrams (mg), about 300 milligrams (mg) to about 750 milligrams (mg), about 300 milligrams (mg) to about 1,000 milligrams (mg), about 300 milligrams (mg) to about 1,500 milligrams (mg), about 400 milligrams (mg) to about 500 milligrams (mg), about 400 milligrams (mg) to about 750 milligrams (mg), about 400 milligrams (mg) to about 1,000 milligrams (mg), about 400 milligrams (mg) to about 1,500 milligrams (mg), about 500 milligrams (mg) to about 750 milligrams (mg), about 500 milligrams (mg) to about 1,000 milligrams (mg), about 500 milligrams (mg) to about 1,500 milligrams (mg), about 750 milligrams (mg) to about 1,000 milligrams (mg), about 750 milligrams (mg) to about 1,500 milligrams (mg), or about 1,000 milligrams (mg) to about 1,500 milligrams (mg). In some embodiments, the amount of S53 protease (e.g., pro-Kumamolisin) administered comprises about 50 milligrams (mg), about 100 milligrams (mg), about 150 milligrams (mg), about 200 milligrams (mg), about 300 milligrams (mg), about 400 milligrams (mg), about 500 milligrams (mg), about 750 milligrams (mg), about 1,000 milligrams (mg), or about 1,500 milligrams (mg). In some embodiments, the amount of S53 protease (e.g., pro-Kumamolisin) administered comprises at least about 50 milligrams (mg), about 100 milligrams (mg), about 150 milligrams (mg), about 200 milligrams (mg), about 300 milligrams (mg), about 400 milligrams (mg), about 500 milligrams (mg), about 750 milligrams (mg), or about 1,000 milligrams (mg). In some embodiments, the amount of S53 protease (e.g., pro-Kumamolisin) administered comprises at most about 100 milligrams (mg), about 150 milligrams (mg), about 200 milligrams (mg), about 300 milligrams (mg), about 400 milligrams (mg), about 500 milligrams (mg), about 750 milligrams (mg), about 1,000 milligrams (mg), or about 1,500 milligrams (mg).

In some embodiments, the amount of legume protein administered comprises about 5 grams (g) to about 50 grams (g). In some embodiments, the amount of legume protein administered comprises about 5 grams (g) to about 10 grams (g), about 5 grams (g) to about 15 grams (g), about 5 grams (g) to about 20 grams (g), about 5 grams (g) to about 25 grams (g), about 5 grams (g) to about 30 grams (g), about 5 grams (g) to about 35 grams (g), about 5 grams (g) to about 40 grams (g), about 5 grams (g) to about 45 grams (g), about 5 grams (g) to about 50 grams (g), about 10 grams (g) to about 15 grams (g), about 10 grams (g) to about 20 grams (g), about 10 grams (g) to about 25 grams (g), about 10 grams (g) to about 30 grams (g), about 10 grams (g) to about 35 grams (g), about 10 grams (g) to about 40 grams (g), about 10 grams (g) to about 45 grams (g), about 10 grams (g) to about 50 grams (g), about 15 grams (g) to about 20 grams (g), about 15 grams (g) to about 25 grams (g), about 15 grams (g) to about 30 grams (g), about 15 grams (g) to about 35 grams (g), about 15 grams (g) to about 40 grams (g), about 15 grams (g) to about 45 grams (g), about 15 grams (g) to about 50 grams (g), about 20 grams (g) to about 25 grams (g), about 20 grams (g) to about 30 grams (g), about 20 grams (g) to about 35 grams (g), about 20 grams (g) to about 40 grams (g), about 20 grams (g) to about 45 grams (g), about 20 grams (g) to about 50 grams (g), about 25 grams (g) to about 30 grams (g), about 25 grams (g) to about 35 grams (g), about 25 grams (g) to about 40 grams (g), about 25 grams (g) to about 45 grams (g), about 25 grams (g) to about 50 grams (g), about 30 grams (g) to about 35 grams (g), about 30 grams (g) to about 40 grams (g), about 30 grams (g) to about 45 grams (g), about 30 grams (g) to about 50 grams (g), about 35 grams (g) to about 40 grams (g), about 35 grams (g) to about 45 grams (g), about 35 grams (g) to about 50 grams (g), about 40 grams (g) to about 45 grams (g), about 40 grams (g) to about 50 grams (g), or about 45 grams (g) to about 50 grams (g). In some embodiments, the amount of legume protein administered comprises about 5 grams (g), about 10 grams (g), about 15 grams (g), about 20 grams (g), about 25 grams (g), about 30 grams (g), about 35 grams (g), about 40 grams (g), about 45 grams (g), or about 50 grams (g). In some embodiments, the amount of legume protein administered comprises at least about 5 grams (g), about 10 grams (g), about 15 grams (g), about 20 grams (g), about 25 grams (g), about 30 grams (g), about 35 grams (g), about 40 grams (g), or about 45 grams (g). In some embodiments, the amount of legume protein administered comprises at most about 10 grams (g), about 15 grams (g), about 20 grams (g), about 25 grams (g), about 30 grams (g), about 35 grams (g), about 40 grams (g), about 45 grams (g), or about 50 grams (g).

In some embodiments, the legume protein is a pea protein. In some embodiments, the pea is a whole pea or a component thereof. In certain embodiments, the pea is a garden pea, a sugar pea, a field pea, or any combination thereof. In certain embodiments, the pea is a garden pea. In certain embodiments, the pea is a sugar pea. In certain embodiments, the pea is a field pea. In certain embodiments, the pea is any combination of a garden pea, a sugar pea, and/or a field pea. In some embodiments, the pea is a standard pea, a commoditized pea, a genetically modified pea, or a combination thereof. In certain embodiments, the pea is a smooth pea, a wrinkled pea, or a combination thereof.

In some embodiments, a composition comprising the legume protein is administered. In some embodiments, a composition comprising the S53 protease (e.g., pro-Kumamolisin) is administered. In some embodiments, a composition comprising the legume protein and the S53 protease (e.g., pro-Kumamolisin) is administered.

In some embodiments, the administering occurs after the subject ingests a foodstuff comprising sugar. In some embodiments, the sugar comprises of sucrose, lactose, maltose, or another di-, tri-, or polysaccharides that contain glucose as a monomer. In certain embodiments, the sugar comprises sucrose. In certain embodiments, the sugar comprises lactose. In certain embodiments, the sugar comprises maltose. In certain embodiments, the sugar comprises di-, tri-, or polysaccharides that contain glucose as a monomer.

In some embodiments, the increase in blood sugar is reduced relative to administering a composition that does not comprise the S53 protease (e.g., pro-Kumamolisin). In some embodiments, the subject self-administers the composition. In some embodiments, the subject is a mammal. In some embodiments, the mammal is a human. In some embodiments, the human has hyperglycemia. In some embodiments, the human has a condition associated with and/or caused by hyperglycemia. In some embodiments, the condition is cardiovascular disease or neuropathy or diabetic nephropathy or retinopathy or cataract or bone and joint problems or teeth and gum infections. In certain embodiments, the condition is cardiovascular disease. In certain embodiments, the condition is a neuropathy. In certain embodiments, the condition is diabetic nephropathy. In certain embodiments, the condition is retinopathy. In certain embodiments, the condition is cataract. In certain embodiments, the condition is bone and joint problems. In certain embodiments, the condition is a tooth infection. In certain embodiments, the condition is a gum infection.

Further provided are methods of reducing the glycemic index of a foodstuff, comprising: providing a legume protein and an S53 protease (e.g., pro-Kumamolisin) to the foodstuff. Further provided is the use of a legume protein and an S53 protease (e.g., pro-Kumamolisin) to reduce the glycemic index of a foodstuff. Furthermore, also provided is a legume protein and an S53 protease (e.g., pro-Kumamolisin) for use in reducing the glycemic index of a foodstuff.

Any of the S53 proteases (e.g., pro-Kumamolisin) described herein can be used in the methods provided. In some embodiments, the S53 protease (e.g., pro-Kumamolisin) comprises an amino acid sequence having at least 85% sequence identity to any one of SEQ ID NOs: 1 and 3-11. In some embodiments, the S53 protease (e.g., pro-Kumamolisin) comprises an amino acid sequence having at least 95% sequence identity to any one of SEQ ID NOs: 1 and 3-11. In some embodiments, the S53 protease (e.g., pro-Kumamolisin) comprises an amino acid sequence having at least 98% sequence identity to any one of SEQ ID NOs: 1 and 3-11. In some embodiments, the active site of the S53 protease comprises amino acid residues E266, F295 or A295, S316, W317, G318, A349, A350 or S350, G351, D352, S353 or D353 or A353 or N353, D367 or E367, G462, G463, T464, S465, and A466 of SEQ ID NO:1.

In some embodiments, the S53 protease (e.g., pro-Kumamolisin) comprises an amino acid sequence of SEQ ID NO: 1. In some embodiments, the S53 protease (e.g., pro-Kumamolisin) comprises an active site comprising amino acid residues E266, F295, S316, W317, G318, A349, A350, G351, D352, S353, D367, G462, G463, T464, S465, and A466 of SEQ ID NO:1. In some embodiments, the S53 protease (e.g., pro-Kumamolisin) comprises an active site comprising one or more amino acid substitutions of residues E266, F295, S316, W317, G318, A349, A350, G351, D352, S353, D367, G462, G463, T464, S465, and A466 of SEQ ID NO:1. In some embodiments, the active site comprises between one and five amino acid substitutions. In some embodiments, the S53 protease (e.g., pro-Kumamolisin) comprises one or more truncations of SEQ ID NO:1, wherein the one or more truncations comprises an N-terminal truncation, a C-terminal truncation, or both an N-terminal and C-terminal truncation.

In some embodiments, the provided amount of pro-Kumamolisin administered comprises about 50 milligrams (mg) to about 1,500 milligrams (mg). In some embodiments, the provided amount of pro-Kumamolisin administered comprises about 50 milligrams (mg) to about 100 milligrams (mg), about 50 milligrams (mg) to about 150 milligrams (mg), about 50 milligrams (mg) to about 200 milligrams (mg), about 50 milligrams (mg) to about 300 milligrams (mg), about 50 milligrams (mg) to about 400 milligrams (mg), about 50 milligrams (mg) to about 500 milligrams (mg), about 50 milligrams (mg) to about 750 milligrams (mg), about 50 milligrams (mg) to about 1,000 milligrams (mg), about 50 milligrams (mg) to about 1,500 milligrams (mg), about 100 milligrams (mg) to about 150 milligrams (mg), about 100 milligrams (mg) to about 200 milligrams (mg), about 100 milligrams (mg) to about 300 milligrams (mg), about 100 milligrams (mg) to about 400 milligrams (mg), about 100 milligrams (mg) to about 500 milligrams (mg), about 100 milligrams (mg) to about 750 milligrams (mg), about 100 milligrams (mg) to about 1,000 milligrams (mg), about 100 milligrams (mg) to about 1,500 milligrams (mg), about 150 milligrams (mg) to about 200 milligrams (mg), about 150 milligrams (mg) to about 300 milligrams (mg), about 150 milligrams (mg) to about 400 milligrams (mg), about 150 milligrams (mg) to about 500 milligrams (mg), about 150 milligrams (mg) to about 750 milligrams (mg), about 150 milligrams (mg) to about 1,000 milligrams (mg), about 150 milligrams (mg) to about 1,500 milligrams (mg), about 200 milligrams (mg) to about 300 milligrams (mg), about 200 milligrams (mg) to about 400 milligrams (mg), about 200 milligrams (mg) to about 500 milligrams (mg), about 200 milligrams (mg) to about 750 milligrams (mg), about 200 milligrams (mg) to about 1,000 milligrams (mg), about 200 milligrams (mg) to about 1,500 milligrams (mg), about 300 milligrams (mg) to about 400 milligrams (mg), about 300 milligrams (mg) to about 500 milligrams (mg), about 300 milligrams (mg) to about 750 milligrams (mg), about 300 milligrams (mg) to about 1,000 milligrams (mg), about 300 milligrams (mg) to about 1,500 milligrams (mg), about 400 milligrams (mg) to about 500 milligrams (mg), about 400 milligrams (mg) to about 750 milligrams (mg), about 400 milligrams (mg) to about 1,000 milligrams (mg), about 400 milligrams (mg) to about 1,500 milligrams (mg), about 500 milligrams (mg) to about 750 milligrams (mg), about 500 milligrams (mg) to about 1,000 milligrams (mg), about 500 milligrams (mg) to about 1,500 milligrams (mg), about 750 milligrams (mg) to about 1,000 milligrams (mg), about 750 milligrams (mg) to about 1,500 milligrams (mg), or about 1,000 milligrams (mg) to about 1,500 milligrams (mg). In some embodiments, the provided amount of pro-Kumamolisin administered comprises about 50 milligrams (mg), about 100 milligrams (mg), about 150 milligrams (mg), about 200 milligrams (mg), about 300 milligrams (mg), about 400 milligrams (mg), about 500 milligrams (mg), about 750 milligrams (mg), about 1,000 milligrams (mg), or about 1,500 milligrams (mg). In some embodiments, the provided amount of pro-Kumamolisin administered comprises at least about 50 milligrams (mg), about 100 milligrams (mg), about 150 milligrams (mg), about 200 milligrams (mg), about 300 milligrams (mg), about 400 milligrams (mg), about 500 milligrams (mg), about 750 milligrams (mg), or about 1,000 milligrams (mg). In some embodiments, the provided amount of pro-Kumamolisin administered comprises at most about 100 milligrams (mg), about 150 milligrams (mg), about 200 milligrams (mg), about 300 milligrams (mg), about 400 milligrams (mg), about 500 milligrams (mg), about 750 milligrams (mg), about 1,000 milligrams (mg), or about 1,500 milligrams (mg).

In some embodiments, the provided amount of legume protein administered comprises about 5 grams (g) to about 60 grams (g). In some embodiments, the provided amount of legume protein administered comprises about 5 grams (g) to about 10 grams (g), about 5 grams (g) to about 15 grams (g), about 5 grams (g) to about 20 grams (g), about 5 grams (g) to about 25 grams (g), about 5 grams (g) to about 30 grams (g), about 5 grams (g) to about 35 grams (g), about 5 grams (g) to about 40 grams (g), about 5 grams (g) to about 45 grams (g), about 5 grams (g) to about 50 grams (g), about 5 grams (g) to about 60 grams (g), about 10 grams (g) to about 15 grams (g), about 10 grams (g) to about 20 grams (g), about 10 grams (g) to about 25 grams (g), about 10 grams (g) to about 30 grams (g), about 10 grams (g) to about 35 grams (g), about 10 grams (g) to about 40 grams (g), about 10 grams (g) to about 45 grams (g), about 10 grams (g) to about 50 grams (g), about 10 grams (g) to about 60 grams (g), about 15 grams (g) to about 20 grams (g), about 15 grams (g) to about 25 grams (g), about 15 grams (g) to about 30 grams (g), about 15 grams (g) to about 35 grams (g), about 15 grams (g) to about 40 grams (g), about 15 grams (g) to about 45 grams (g), about 15 grams (g) to about 50 grams (g), about 15 grams (g) to about 60 grams (g), about 20 grams (g) to about 25 grams (g), about 20 grams (g) to about 30 grams (g), about 20 grams (g) to about 35 grams (g), about 20 grams (g) to about 40 grams (g), about 20 grams (g) to about 45 grams (g), about 20 grams (g) to about 50 grams (g), about 20 grams (g) to about 60 grams (g), about 25 grams (g) to about 30 grams (g), about 25 grams (g) to about 35 grams (g), about 25 grams (g) to about 40 grams (g), about 25 grams (g) to about 45 grams (g), about 25 grams (g) to about 50 grams (g), about 25 grams (g) to about 60 grams (g), about 30 grams (g) to about 35 grams (g), about 30 grams (g) to about 40 grams (g), about 30 grams (g) to about 45 grams (g), about 30 grams (g) to about 50 grams (g), about 30 grams (g) to about 60 grams (g), about 35 grams (g) to about 40 grams (g), about 35 grams (g) to about 45 grams (g), about 35 grams (g) to about 50 grams (g), about 35 grams (g) to about 60 grams (g), about 40 grams (g) to about 45 grams (g), about 40 grams (g) to about 50 grams (g), about 40 grams (g) to about 60 grams (g), about 45 grams (g) to about 50 grams (g), about 45 grams (g) to about 60 grams (g), or about 50 grams (g) to about 60 grams (g). In some embodiments, the provided amount of legume protein administered comprises about 5 grams (g), about 10 grams (g), about 15 grams (g), about 20 grams (g), about 25 grams (g), about 30 grams (g), about 35 grams (g), about 40 grams (g), about 45 grams (g), about 50 grams (g), or about 60 grams (g). In some embodiments, the provided amount of legume protein administered comprises at least about 5 grams (g), about 10 grams (g), about 15 grams (g), about 20 grams (g), about 25 grams (g), about 30 grams (g), about 35 grams (g), about 40 grams (g), about 45 grams (g), or about 50 grams (g). In some embodiments, the provided amount of legume protein administered comprises at most about 10 grams (g), about 15 grams (g), about 20 grams (g), about 25 grams (g), about 30 grams (g), about 35 grams (g), about 40 grams (g), about 45 grams (g), about 50 grams (g), or about 60 grams (g).

In some embodiments, the legume protein is a pea protein. In some embodiments, the pea is a whole pea or a component thereof. In certain embodiments, the pea is a garden pea, a sugar pea, a field pea, or any combination thereof. In certain embodiments, the pea is a garden pea. In certain embodiments, the pea is a sugar pea. In certain embodiments, the pea is a field pea. In certain embodiments, the pea is any combination of a garden pea, a sugar pea, and/or a field pea. In some embodiments, the pea is a standard pea, a commoditized pea, a genetically modified pea, or a combination thereof. In certain embodiments, the pea is a smooth pea, a wrinkled pea, or a combination thereof.

In some embodiments, a composition comprising the legume protein is added. In some embodiments, a composition comprising the S53 protease (e.g., pro-Kumamolisin) is added. In some embodiments, a composition comprising the legume protein and the S53 protease (e.g., pro-Kumamolisin) is added.

In some embodiments, the foodstuff comprises sugar. In some embodiments, the sugar comprises of sucrose, lactose, maltose, or another di-, tri-, or polysaccharides that contain glucose as a monomer. In certain embodiments, the condition is cardiovascular disease. In certain embodiments, the condition is a neuropathy. In certain embodiments, the condition is diabetic nephropathy. In certain embodiments, the condition is retinopathy. In certain embodiments, the condition is cataract. In certain embodiments, the condition is bone and joint problems. In certain embodiments, the condition is a tooth infection. In certain embodiments, the condition is a gum infection.

In some embodiments, the method further comprises administering a low-carbohydrate diet. In certain embodiments, the low-carbohydrate diet comprises a diet wherein less than 65% of total energy intake per day (e.g., total calorie intake per day) is from carbohydrates. In certain embodiments, the low-carbohydrate diet comprises a diet wherein less than 60% of total energy intake per day (e.g., total calorie intake per day) is from carbohydrates. In certain embodiments, the low-carbohydrate diet comprises a diet wherein less than 50% of total energy intake per day (e.g., total calorie intake per day) is from carbohydrates. In certain embodiments, the low-carbohydrate diet comprises a diet wherein less than 45% of total energy intake per day (e.g., total calorie intake per day) is from carbohydrates. In certain embodiments, the low-carbohydrate diet comprises a diet wherein less than 40% of total energy intake per day (e.g., total calorie intake per day) is from carbohydrates. In certain embodiments, the low-carbohydrate diet comprises a diet wherein less than 35% of total energy intake per day (e.g., total calorie intake per day) is from carbohydrates. In certain embodiments, the low-carbohydrate diet comprises a diet wherein less than 30% of total energy intake per day (e.g., total caloric intake per day) is from carbohydrates. In some embodiments, the method further comprises reducing the caloric intake (e.g., total caloric intake per day) obtained from carbohydrates. In certain embodiments, reducing the caloric intake (e.g., total caloric intake per day) obtained from carbohydrates comprises reducing the amount (e.g., grams or calories) of carbohydrate intake as measured by or compared to the carbohydrate intake prior to administering a protease or composition comprising a protease to the subject.

In certain embodiments, the amount (grams or calories) of carbohydrates is reduced by about 10% to about 65%. In certain embodiments, the amount (grams or calories) of carbohydrates is reduced by about 10% to about 20%, about 10% to about 30%, about 10% to about 40%, about 10% to about 50%, about 10% to about 65%, about 20% to about 30%, about 20% to about 40%, about 20% to about 50%, about 20% to about 65%, about 30% to about 40%, about 30% to about 50%, about 30% to about 65%, about 40% to about 50%, about 40% to about 65%, or about 50% to about 65%. In certain embodiments, the amount (grams or calories) of carbohydrates is reduced by about 10%, about 20%, about 30%, about 40%, about 50%, or about 60%. In certain embodiments, the amount (grams or calories) of carbohydrates is reduced by at least about 10%, about 20%, about 30%, about 40%, 50%, 60% or about 65%.

Carbohydrates generally refer to and include to aldehyde or ketone compounds substituted with multiple hydroxyl groups, of the general formula (CH2O)ₙ, wherein n is 3-30, as well as their oligomers and polymers. Carbohydrates can be substituted or deoxygenated at one or more positions. Carbohydrates include and/or encompass monosaccharides, disaccharides, oligosaccharides, and polysaccharides. Carbohydrates further include unmodified carbohydrates, carbohydrate derivatives, substituted carbohydrates, and modified carbohydrates. The carbohydrate derivatives or substituted carbohydrates include substituted and unsubstituted monosaccharides, disaccharides, oligosaccharides, and polysaccharides. A carbohydrate derivative or substituted carbohydrate can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. As also used herein, in any instance or embodiment described herein, “comprising” may be replaced with “consisting essentially of” and/or “consisting of”. used herein, in any instance or embodiment described.

### Examples

### Example 1—Lowering Blood Glucose Using a Legume Protein and Pro-Kumamolisin

Protease Production

The DNA sequence (SEQ ID NO:2) of the acid protease of interest was cloned into the expression vector pET29b(+) for protease production in E. coli. The completed DNA construct was transformed into an expression strain of E. coli (BL21) and grown at 37 degrees Celsius in Terrific Broth using a baffled shake flask for 4-6 hours until the cell density (measured using OD 600) reaches 0.6. The cultures were then induced with 0.5 mM of IPTG for protease expression. The culture was grown at 30 degrees Celsius for 12 hours post induction before harvesting. The harvested cells were lysed using sonication and the protease was purified from cell lysate using IMAC chromatography.

Application of Pro-Kumamolisin in a Pea Protein Drink

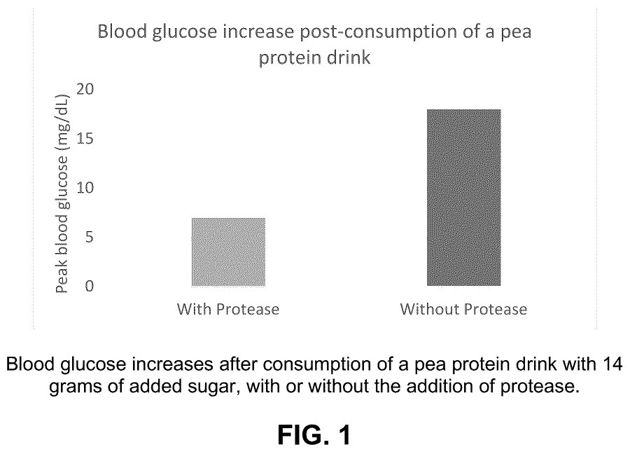

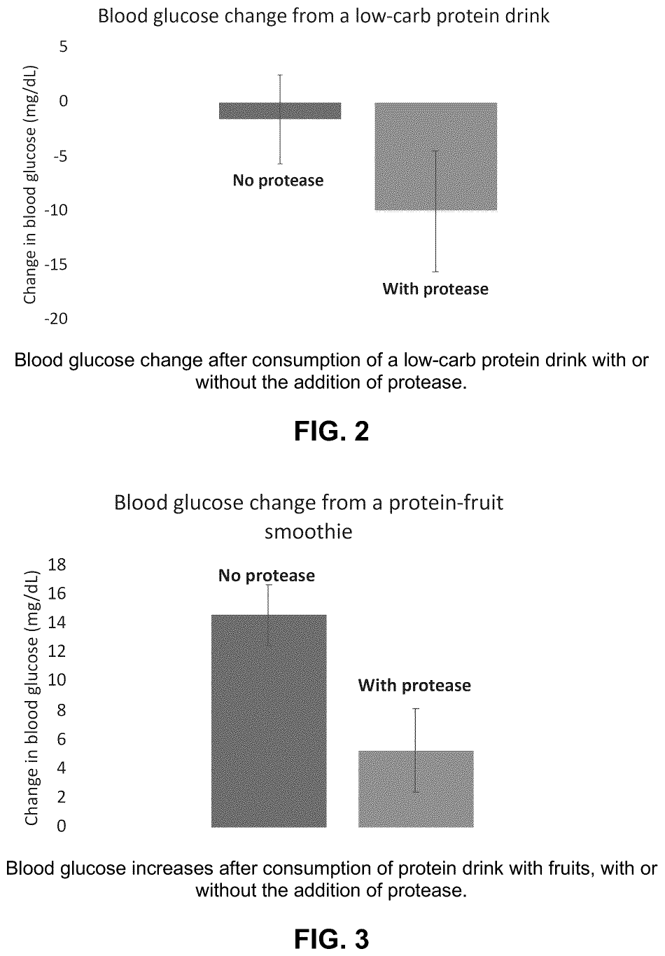

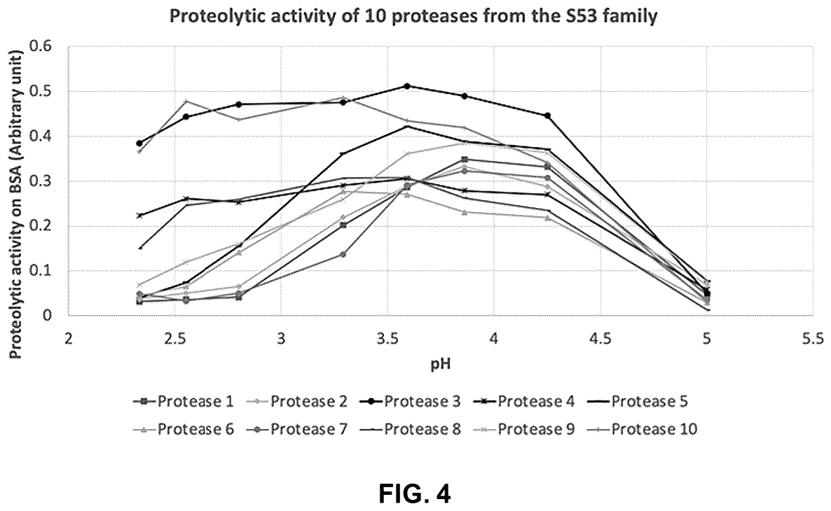

30 grams of pea protein was dissolved in 330 mL of water. 14 grams of sucrose was then dissolved followed by 200 mg of acid protease (SEQ ID NO. 1). The protein drink of the study can be consumed as is or formulated with other commonly used ingredients found in protein shakes. As shown in FIG. 1 the addition of protease can lower the post-consumption blood glucose spike by more half at the blood glucose peak period. FIG. 1 shows the comparison of post-consumption blood glucose levels resulting from the intake of (i) a protein drink comprising pro-Kumamolisin and pea protein, compared against (ii) a protein drink without pro-Kumamolisin and pea protein.

### Example 2—Activity of S53 Proteases at Low pH

Generally, the S53 proteases described and used herein should have the capability to thoroughly digest proteins (e.g., legume protein) in the stomach's acidic environment. Accordingly, the S53 proteases used and described herein should possess activity throughout the entire post-prandial pH range of the stomach environment. pH profile data was generated for 10 representative S53 proteases (encompassing SEQ ID NOs: 1 and 3-11). The representative S53 proteases showed optimal activity (e.g., 100% or substantially active) throughout a pH range between 2.5 to 4.5. FIG. 6 shows proteolytic activity of S53 proteases 1-10 (P1-P10) wherein S53 proteases 1-10 across pH 2 to 5.

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

SEQUENCES

| SEQ ID NO | SEQUENCE | DESCRIPTION |
|---|---|---|
| 1 | MSDMEKPWKEEEKREVLAGHARRQAPQAVDKGPVTGDQRI | Protein |
| | SVTVVLRRORGDELEAHVERQAALAPHARVHLEREAFAAS | Kumamolisin |
| | HGASLDDFAEIRKFAEAHGLTLDRAHVAAGTAVLSGPVDA | precursor |
| | VNQAFGVELRHFDHPDGSYRSYVGDVRVPASIAPLIEAVL | [Bacillus |
| | GLDTRPVARPHFRLRRRAEGEFEARSQSAAPTAYTPLDVA | sp. MN-32] |
| | QAYQFPEGLDGQGQCIAIIELGGGYDETSLAQYFASLGVS | Protease 3 |
| | APQVVSVSVDGATNQPTGDPNGPDGEVELDIEVAGALAPG | |
| | AKIAVYFAPNTDAGFLNAITTAVHDPTHKPSIVSISWGGP | |
| | EDSWAPASIAAMNRAFLDAAALGVTVLAAAGDSGSTDGEQ | |
| | DGLYHVDFPAASPYVLACGGTRLVASAGRIERETVWNDGP | |
| | DGGSTGGGVSRIFPLPSWQERANVPPSANPGAGSGRGVPD | |
| | VAGNADPATGYEVVIDGETTVIGGTSAVAPLFAALVARIN | |
| | QKLGKPVGYLNPTLYQLPPEVFHDITEGNNDIANRARIYQ | |
| | AGPGWDPCTGLGSPIGIRLLQALLPSASQAQP | |
| 2 | ATGAGCGATATGGAAAAACCGTGGAAAGAAGAAGAAAAAC | DNA |
| | GCGAAGTTCTGGCAGGTCATGCACGTCGTCAGGCACCGCA | Kumamolisin |
| | GGCAGTTGATAAAGGTCCGGTTACCGGTGATCAGCGTATT | precursor |
| | AGCGTTACCGTTGTTCTGCGTCGTCAGCGTGGTGATGAAC | [Bacillus |
| | TGGAAGCACATGTTGAACGTCAGGCAGCACTGGCACCGCA | sp. MN-32] |
| | TGCACGTGTTCATCTGGAACGTGAAGCATTTGCAGCAAGC | Protease 3 |
| | CATGGTGCAAGCCTGGATGATTTTGCAGAAATTCGTAAAT | |
| | TTGCCGAAGCGCATGGTCTGACCCTGGATCGTGCCCATGT | |
| | TGCAGCAGGTACAGCAGTTCTGAGCGGTCCGGTTGATGCA | |
| | GTTAATCAGGCATTTGGTGTTGAACTGCGTCATTTTGATC | |
| | ATCCTGATGGTAGCTATCGTAGCTATGTTGGTGATGTTCG | |
| | TGTTCCGGCAAGCATTGCACCGCTGATTGAAGCAGTTTTA | |
| | GGTCTGGATACCCGTCCGGTTGCACGTCCGCATTTTCGTC | |
| | TGCGTCGCCGTGCAGAAGGTGAATTTGAAGCACGTAGCCA | |
| | GAGCGCAGCACCGACCGCATATACACCGCTGGATGTTGCA | |
| | CAGGCATATCAGTTTCCGGAAGGCCTGGATGGTCAGGGTC | |
| | AGTGTATTGCAATTATTGAATTAGGTGGTGGCTATGATGA | |
| | AACCAGCCTGGCACAGTATTTTGCCAGCCTGGGTGTTAGC | |
| | GCTCCGCAGGTTGTTAGCGTTAGCGTGGATGGTGCAACCA | |
| | ATCAGCCGACAGGTGATCCGAATGGTCCGGATGGTGAAGT | |
| | TGAACTGGATATTGAAGTTGCCGGTGCGCTGGCACCGGGT | |
| | GCAAAAATTGCAGTTTATTTTGCACCGAATACCGATGCCG | |
| | GTTTTCTGAATGCAATTACCACCGCAGTTCATGATCCGAC | |
| | ACATAAACCGAGCATTGTGAGCATTAGCTGGGGTGGTCCG | |
| | GAAGATAGCTGGGCACCAGCCAGCATTGCAGCCATGAATC | |
| | GTGCATTTCTGGATGCAGCCGCACTGGGTGTGACCGTGCT | |
| | GGCAGCAGCCGGTGATAGCGGTAGCACCGATGGTGAACAG | |
| | GATGGTCTGTATCATGTTGATTTTCCGGCAGCGAGCCCGT | |
| | ATGTTCTGGCATGTGGTGGCACCCGTCTGGTGGCAAGCGC | |
| | AGGTCGTATTGAACGTGAAACCGTTTGGAATGATGGTCCT | |
| | GATGGCGGTTCAACCGGTGGTGGTGTTAGCCGTATTTTTC | |
| | CGCTGCCGAGCTGGCAAGAACGTGCAAATGTTCCGCCTAG | |
| | CGCAAATCCTGGTGCAGGTAGCGGTCGTGGTGTTCCGGAT | |
| | GTTGCCGGTAATGCAGATCCGGCAACCGGTTATGAAGTTG | |
| | TTATTGATGGTGAAACCACCGTGATTGGTGGTACAAGCGC | |
| | AGTGGCACCGCTGTTTGCAGCCCTGGTTGCCCGTATTAAT | |
| | CAGAAACTGGGTAAACCGGTTGGTTATCTGAATCCGACAC | |
| | TGTATCAGCTGCCTCCGGAAGTTTTTCATGATATTACCGA | |
| | AGGCAACAACGATATTGCCAATCGTGCACGTATTTATCAG | |
| | GCAGGTCCTGGTTGGGATCCGTGTACCGGTCTGGGTAGCC | |
| | CGATTGGTATTCGTCTGCTGCAGGCACTGCTGCCGAGTGC | |
| | AAGCCAGGCACAGCCGTGA | |
| 3 | MSEPVPAAARRTIPGSERPPVDTAAAARQAVPADTRVEAT | Protease 1 |
| | VVLRRRAELPDGPGLLTPAELAERHGADPADVELVTRTLT | |
| | GLGVEVTAVDAASRRLRVAGPAGVLAEAFGTSLAQVSTPD | |
| | PSGAQVTHRYRAGALSVPAELDGVVTAVLGLDDRPQARAR | |
| | FRVATAAAASAGYTPIELGRVYSFPEGSDGSGQTIAIIEL | |
| | GGGFAQSELDTYFAGLGISGPTVTAVGVDGGSNVAGRDPQ | |
| | GADGEVLLDIEVAGALAPGADVVVYFAPNTDAGFLDAVAQ | |
| | AAHATPTPAAISISWGGSEDTWTGQARTAFDAALADAAAL | |
| | GVTTTVAAGDDGSTDRATDGKSHVDFPASSPHALACGGTH | |
| | LDANATTGAVTSEVVWNNGAGKGATGGGVSTVFAQPSWQA | |
| | SAGVPDGPGGKPGRGVPDVSAVADPQTGYRIRVDGQDLVI | |
| | GGTSAVAPLWAALVARLVQAGRAKLGLLQPKLYAAPTAFR | |
| | DITEGDNGAYRAGPGWDACTGLGVPVGTALASALS | |
| 4 | MADDSSPTTAADRPTLPGSARRPVAAAQAAGPLDDAAPLE | Protease 2 |
| | VTLVLRRRTALPAGTGRPAPMGRAEFAETHGADPADAETV | |
| | TAALTAEGLRITAVDLPSRRVQVAGDVATFSRVFGVSLSR | |
| | VESPDPVADRLVPHRQRSGDLAVPAPLAGVVTAVLGLDDR | |
| | PQARALFRPAAAVDTTFTPLELGRVYRFPSGTDGRGQRLA | |
| | ILELGGGYTQADLDAYWTTIGLADPPTVTAVGVDGAANAP | |
| | EGDPNGADGEVLLDIEVAGALAPGADLVVYFAPNTDRGFL | |
| | DALSTAVHADPTPTAVSISWGQNEDEWTAQARTAMDEALA | |
| | DAAALGVTVCAAAGDDGSTDNAPDGQAHVDFPASSPHALA | |
| | CGGTTLRADPDTGEVSSETVWFHGTGQGGTGGGVSAVFAV | |
| | PDWQDGVRVPGDADTGRHGRGVPDVSADADPSTGYQVRVD | |
| | GTDAVFGGTSAVSPLWSALTCRLAEALGQRPGLLQPLIYA | |
| | GLSAGEVAAGFRDVTSGSNGAYDAGPGWDPCTGLGVPDGE | |
| | ALLVRLRTALG | |
| 5 | MANRKMFPNSVIAIPTSGVTAHGLIVSAADPQSRDEKMDV | Protease 4 |
| | SFSLGIPPALEKELEERVDKGETIPPQELTTKYAVDPTAA | |
| | GTLQTWLKKEGFTITGVTPDRTTIYASAPASQVEASLGVH | |
| | TVRVTREGQTYTAASDVPSLPEDIGGAVVNIGGLQPYRQA | |
| | RKHLRSYIQTTPEADGEEPAIANAPPYLVPEILKAYDGAR | |
| | LGLTGKGQEIAILIDTVPLDTDLTSFWTANGVAGSLARIT | |
| | KINVKGGALPTPSGEETLDAEWASTIAPDANVRIYASGTL | |
| | SFIDLDRALDRIYADALAQPKLRIVSISLGLSEAYMAKGE | |
| | VDAEEARFVRFAALGVNVFVSTGDAGSNPGPDGHHANGPL | |
| | AAEWMSTSPHVVAVGGTSLRLANNGQVASETGWTGSGGGK | |
| | SNFQPRPAWQQGHGVPAGNQRMVPDVGAAADPNEGALVIL | |
| | NGQRLQYGGTSWSAPIWAGLCALINEARQNNHKTPLPYLN | |
| | SLIYPMIGSNCFRDELTGSNGAYSCGPGYDLVTGIGSPDL | |
| | KQLAAKLA | |
| 6 | MAGVNEPYNAREDGIPLKSSARAVVPGVKLHGPTDGASRL | Protease 5 |
| | EITVVLRRRTELPSAAADGHLTAAELASEYGASDDDVRLA | |
| | TEVFTRLGADVVESDPASRRLRLSGTVEQLSSIFGTTLED | |
| | ATSTAPDGATVHYRHRLGELRIPAELNGIVIAVLGLDDRP | |
| | QARAHFRMLPRTTAGTSYSPVELGRVYGFPDGTDGSGQTV | |
| | AIIELGGGYAQADLDAYFAGLGLATPQITSIGVDGGANQG | |
| | GNDPQGADGEVLLDIEVVGALAPKAAIQVYFAPNTDAGFL | |
| | DAVVAATKAAPCAISISWGQSEDQWTAQARDAFDQALADA | |
| | AALGITTTVAAGDRGSSDGAADGKAHVDFPASSPHALACG | |
| | GTRLEADPATGAIRSETVWNEGPDSATGGGYSKVFPRPSW | |
| | QSPSAGKSGRGVPDVSAVADPQTGYRIRVDGKDMVIGGTS | |
| | AVAPLWAALIARFAQAGNRRFGLIQPSLYAVSSGFRDVTV | |
| | GDNGSYHAGPGWDACTGLGTPDGAALLAALKG | |
| 7 | MSTRAARTTPSALADLRNEPRSPLPGSEKAALADTPATTA | Protease 6 |
| | AGIKPLRATAVAKAKPASSRKKITVSVVVPRTKPVTQAAV | |
| | AGKHLTRAQFKSSHAAAPASVKAVQKFAKAFNLVSKAEPA | |
| | RSTVHLTGTVKDMQDAFGVTLQEHTVGAKTLRIRQGAIYL | |
| | PDSVLPHVQAVLGLDNRPQAKPHYRVGKARAAASTSFTPP | |
| | QLAQLYGFPTSAKATGQTIALIELGGGFRQADITAYFKSL | |
| | GIAAPSVKAVLVDGGKNAPSNANGADGEVMLDIEVAAAVA | |
| | PGANIAVYFAPNTDQGFVDAIATAAHDTTNKPTIISISWG | |
| | GPESSWTSQALTALDNACKDAAALGITVTAAAGDDGSDDG | |
| | VGDGKKHVDFPASSPNVLACGGTKLVASNGAITSEVVWNE | |
| | TANKEGATGGGISTAFPQPTWQKSIAATKSGRGVPDVAGD | |
| | ADPTTGYQVRVDGQNMVIGGTSAVAPLWAGLIALSNATNK | |
| | NAAGLPQAKLYSTTGQKAFRDITSGNNGAFKAAKGWDPCT | |
| | GLGSPKAASIITLLATKSSAKKKTSRAKA | |
| 8 | MESIMPSQPSSIPVRGSERAALPTAHVVGPAASDERLEVT | Protease 7 |
| | LRVRPRAQLHASASEAQSLRPPGERSYLSREQLASAHGAA | |
| | PEDIAKVEAFAQSHGLQVVLTSAARRCVIVSGTVAALESA | |
| | FAVKLQQYRFDGGSYRGRVGPVFVSPEIGDIVEGVFGLDD | |
| | RPQAIAHFKRSAHAVRAEDGAAPHAGGASFTPPQLAKLYN | |
| | YPGDTDGTGQCIGIVEFGGAIRAADIRAYFKELGLPAPHV | |
| | NTVLVDHAHMRSDDADAEVMLDIEVAAAIAPKAQIVVYFA | |
| | PNTSQGFIDAFTHAIHDTVHKPSVISVSWGGPEKDWSAQI | |
| | KTQLDQVFQDAAALGVTICAASGDAGSSDENPDALASIGL | |
| | TPDGLSHADFPASSPFALACGGTKLVASASAITSETVWNE | |
| | DPVRSATGGGISDFFDVPGYQATANIPVSANPGGRKGRGV | |
| | PDIAADADPATGYLVRVHGQDAVIGGTSAVAPLMAGLVAL | |
| | LNHKLGHPVGFLNPLLYRTAGITRDITQGNNGAYAAGKGW | |
| | DACTGLGVPDGAKLLDALM | |
| 9 | MPQSQNRVVVRGSERQPMPKAHSQHALPPTERLEVTVRLR | Protease 8 |
| | PKAALASAAASSHAMADVPPSQRTYLSREELAAQCGASED | |
| | DAQAVADFAHAHGLVVIHTDLARRSVLLAGTAADFGAAFG | |
| | TQLHQYSSPEGTYRGRTGTVTVPAPLADIVQGVFGLDDRR | |
| | QAEPHFQVRPGPTPAPGAIVARAAGQSFTPPQLAQLYDFP | |
| | GGLDGTGQTIAVIELGGGFKPADLKAYFTGLNLPVPTVKV | |
| | VSVNGGRNQPTNANSADGEVLLDIEVAAAVAPRAHLVVYF | |
| | APNTSQGFLNAITTAVHDKVNNPGIISISWGGPESTWTGQ | |
| | AMDQFDQAFQEAAMLGVTVCVAAGDNGSADGVADGQPHAD | |
| | FPASSPFALACGGTKLTASGPTISSEVVWNEGPNSATGGG | |
| | LSAHFPVPAYQQQLKFPTPPAGAKAGRGLPDVAGDADPNT | |
| | GYQVRVDGQNLVIGGTSAVAPLWAGLLALLNQKLPKPVGF | |
| | LNPLLYGSLAGQGVTRDITSGNNGAFAAGPGWDACTGWGS | |
| | PVGGKLLAALQGGAAVA | |
| 10 | MSKHPLMGSERAPFDGAQSVGKADPAERLEVTVLVRRGSS | Protease 9 |
| | DALRTRVSKLVAGNASDGHIQREDFAQQFGAAPNDMSAVR | |
| | NFASQHGLSVVEEHAARRTVILSGTVAQFNDAFDVDLQQF | |
| | EHAGGSYRGRTGPVHLPDELSGVVDAVLGLDNRPQARPHF | |
| | RSRPPQGNVHWQSSRTGTTSSTPLQLASLYDFPAGTGQGQ | |
| | CIAIIELGGGYRPADLKAYFSKLGIASPKVTTVSVDHGKN | |
| | HPTGDANGPDGEVMLDIEIAGAIAPGAHIAVYFAPNTDAG | |
| | FLDAVTTAIHDTIRKPSVISISWGGPESAWTEQAMTAFDQ | |
| | AFQAAAALGITVCVASGDNGSGDGVNDGADHVDFPASSPY | |
| | ALACGGTSVQAGKGAIAKETVWNDGANGGASGGGVSSFFA | |
| | LPAWQEGLQAARAKGGTGALQMRGVPDVAGNADPATGYDV | |
| | RVDGSDMVIGGTSAVAPLWAGLVARINAGKNSPAGYLNPK | |
| | LYKTAAGLTDITQGNNGDFVASAGWDACTGLGRPDGNKLA | |
| | GTFG | |
| 11 | MDETNFTSTNGSPQYIPVTGSARAIVPGATHAGHTDDNEV | Protease 10 |
| | LSVTLQLRRPSADELTAHVEALGTTPPANRKHMTHDEFEA | |
| | SHGASDDDLNLVTAFATEQGLSVERINKAAATVHVSGTAG | |
| | AFNKAFHVQLGNYQHPDFTYRGYDGPVHIPAHLTDIVTGV | |
| | LGLDNRPQAKPHFRVYQEAAVRSNALAAPISYTPTQVAAL | |
| | YNFPTNVDCSGQCIGIIELGGGYSKSNLDQYFASLGVPTP | |
| | TITSVSVDGGQNQPTGSPNGPDGEVDLDIEVAASVAPGAH | |
| | IAVYFAPNTDAGFLDAITTAVHDKTNKPSVISISWGGPEM | |
| | SWTTQAMQAMNNAMQSAAALGVTITVAAGDNGSTDGVNDG | |
| | SFHVDFPASAPYALACGGTHLVGSGSTIESETVWNDGANG | |
| | GATGGGVSSVFPVPSWQQKANVPPSANPGAGTGRGVPDVS | |
| | GDADPATGYQVLVDGQQFPIGGTSAVAPLWAGLVALANQT | |
| | LGKPVGYINPLLYSIPAQDNAFHDITQGNNDPNQTGQVYP | |
| | AGPGWDACTGLGSPNGTLLIQALGQIG | |
| 12 | ATGAGCGAACCTGTTCCGGCAGCAGCACGTCGTACCATTC | Protease 1 |
| | CGGGTAGCGAACGTCCGCCTGTTGATACCGCAGCAGCAGC | |
| | CCGTCAGGCAGTTCCTGCAGATACCCGTGTTGAAGCAACC | |
| | GTTGTTCTGCGTCGTCGTGCAGAACTGCCGGATGGTCCGG | |
| | GTCTGCTGACACCGGCAGAACTGGCAGAACGTCATGGTGC | |
| | AGATCCGGCAGATGTTGAACTGGTTACCCGTACACTGACC | |
| | GGTCTGGGTGTTGAAGTTACCGCAGTTGATGCAGCAAGCC | |
| | GTCGTCTGCGTGTTGCCGGTCCGGCAGGCGTTCTGGCAGA | |
| | AGCATTTGGCACCAGCCTGGCACAGGTTAGCACACCGGAT | |
| | CCGAGCGGTGCCCAGGTTACCCATCGTTATCGTGCCGGTG | |
| | CACTGAGCGTTCCAGCCGAACTGGATGGTGTTGTGACCGC | |
| | AGTTCTGGGTTTAGATGATCGTCCGCAGGCACGTGCGCGT | |
| | TTTCGTGTTGCAACGGCAGCCGCAGCAAGCGCAGGTTATA | |
| | CCCCGATTGAACTGGGTCGTGTTTATAGCTTTCCGGAAGG | |
| | TAGTGATGGTAGCGGTCAGACCATTGCAATTATTGAATTA | |
| | GGTGGTGGTTTTGCACAGAGTGAACTGGATACCTATTTTG | |
| | CAGGTCTGGGTATTAGCGGTCCGACCGTTACAGCAGTTGG | |
| | TGTTGATGGTGGTAGCAATGTTGCAGGTCGTGATCCGCAG | |
| | GGTGCAGATGGTGAAGTTCTGCTGGATATTGAAGTTGCGG | |
| | GTGCACTGGCACCGGGTGCCGATGTTGTTGTTTATTTTGC | |
| | ACCGAATACCGATGCAGGTTTTCTGGATGCAGTTGCACAG | |
| | GCAGCACATGCAACCCCGACTCCGGCAGCCATTAGCATTA | |
| | GCTGGGGTGGTAGCGAAGATACCTGGACAGGTCAGGCACG | |
| | TACCGCCTTTGATGCGGCACTGGCAGATGCAGCCGCACTG | |
| | GGTGTTACCACCACCGTTGCAGCCGGTGATGATGGTAGTA | |
| | CCGATCGTGCAACCGATGGTAAAAGCCATGTTGATTTTCC | |
| | GGCAAGCAGTCCGCATGCACTGGCCTGTGGTGGCACCCAT | |
| | CTGGATGCCAATGCAACCACCGGTGCAGTTACCAGCGAAG | |
| | TTGTTTGGAATAATGGTGCAGGTAAAGGTGCAACCGGTGG | |
| | CGGTGTTAGCACCGTTTTTGCCCAGCCGAGCTGGCAGGCA | |
| | AGTGCCGGTGTTCCGGATGGCCCTGGTGGTAAACCTGGTC | |
| | GTGGTGTGCCGGATGTTAGCGCAGTTGCCGATCCGCAGAC | |
| | CGGTTATCGTATTCGTGTGGATGGTCAGGATCTGGTTATT | |
| | GGTGGTACAAGCGCAGTGGCACCGCTGTGGGCAGCACTGG | |
| | TTGCACGTCTGGTTCAGGCAGGTCGCGCAAAACTGGGCCT | |
| | GCTGCAGCCGAAACTGTATGCAGCACCGACCGCATTTCGT | |
| | GATATTACCGAAGGTGATAATGGCGCATATCGTGCAGGTC | |
| | CTGGTTGGGATGCATGTACAGGCCTGGGCGTTCCGGTTGG | |
| | CACCGCACTGGCGAGCGCACTGAGTCTCGAGCACCACCAC | |
| | CACCACCACTGA | |
| 13 | ATGGCCGATGATAGCAGCCCGACCACCGCAGCAGATCGTC | Protease 2 |
| | CGACACTGCCTGGTAGCGCACGTCGTCCGGTTGCAGCAGC | |
| | ACAGGCAGCAGGTCCGCTGGATGATGCAGCACCGCTGGAA | |
| | GTTACCCTGGTTCTGCGTCGTCGTACCGCACTGCCAGCAG | |
| | GCACAGGTCGTCCGGCACCGATGGGTCGTGCAGAATTTGC | |
| | AGAAACCCATGGTGCAGATCCGGCAGATGCCGAAACCGTT | |
| | ACCGCAGCACTGACCGCAGAAGGTCTGCGTATTACCGCAG | |
| | TTGATCTGCCGAGCCGTCGTGTTCAGGTTGCCGGTGATGT | |
| | TGCAACCTTTAGCCGTGTTTTTGGTGTTAGCCTGAGCCGT | |
| | GTTGAAAGCCCTGATCCGGTTGCCGATCGTCTGGTTCCGC | |
| | ATCGTCAGCGTAGCGGTGATCTGGCAGTTCCTGCTCCGCT | |
| | GGCAGGCGTTGTGACCGCAGTTCTGGGTTTAGATGATCGT | |
| | CCGCAGGCACGTGCACTGTTTCGTCCTGCAGCAGCCGTTG | |
| | ATACCACCTTTACTCCGCTGGAACTGGGTCGTGTTTATCG | |
| | TTTTCCGAGCGGTACAGATGGTCGTGGTCAGCGTCTGGCA | |
| | ATTCTGGAATTAGGTGGTGGTTATACCCAGGCAGATCTGG | |
| | ATGCATATTGGACCACCATTGGTCTGGCAGATCCGCCTAC | |
| | CGTTACAGCAGTTGGTGTTGATGGTGCAGCAAATGCACCG | |
| | GAAGGTGATCCGAATGGTGCCGATGGTGAAGTTCTGCTGG | |
| | ATATTGAAGTTGCGGGTGCACTGGCACCGGGTGCCGATCT | |
| | GGTTGTTTATTTTGCACCGAATACCGATCGTGGTTTTCTG | |
| | GATGCCCTGAGCACCGCAGTGCATGCCGATCCGACACCGA | |
| | CCGCAGTGAGCATTAGCTGGGGTCAGAATGAAGATGAATG | |
| | GACCGCACAGGCACGTACCGCAATGGATGAAGCACTGGCA | |
| | GATGCAGCCGCACTGGGTGTTACCGTTTGTGCAGCAGCGG | |
| | GTGATGATGGTAGCACAGATAACGCACCGGATGGTCAGGC | |
| | ACATGTTGATTTTCCGGCAAGCAGTCCGCATGCGCTGGCA | |
| | TGTGGTGGTACAACCCTGCGTGCGGATCCGGATACCGGTG | |
| | AAGTTAGCAGCGAAACCGTGTGGTTTCATGGCACCGGTCA | |
| | AGGTGGTACTGGTGGTGGTGTGAGCGCAGTTTTTGCAGTT | |
| | CCGGATTGGCAGGATGGTGTTCGTGTTCCGGGTGATGCAG | |
| | ATACCGGTCGTCATGGTCGCGGTGTTCCGGATGTTAGCGC | |
| | AGATGCTGATCCGAGTACCGGTTATCAGGTTCGTGTGGAT | |
| | GGTACGGATGCAGTGTTTGGTGGCACCAGCGCAGTTAGTC | |
| | CGCTGTGGTCTGCACTGACCTGTCGTCTGGCCGAAGCGCT | |
| | GGGACAGCGTCCGGGTCTGCTGCAGCCGCTGATTTATGCA | |
| | GGTCTGAGCGCAGGCGAAGTTGCAGCCGGTTTTCGTGATG | |
| | TTACCAGCGGTAGCAATGGTGCATACGATGCAGGTCCTGG | |
| | TTGGGATCCGTGCACCGGTCTGGGTGTGCCGGATGGCGAA | |
| | GCACTGCTGGTTCGTCTGCGTACAGCACTGGGCCTCGAGC | |
| | ACCACCACCACCACCACTGA | |
| 14 | ATGGCCAACCGTAAAATGTTTCCGAATAGCGTTATTGCAA | Protease 4 |
| | TTCCGACCAGCGGTGTTACCGCACATGGTCTGATTGTTAG | |
| | CGCAGCAGATCCGCAGAGCCGTGATGAAAAAATGGATGTT | |
| | AGCTTTAGCCTGGGTATTCCGCCTGCACTGGAAAAAGAAC | |
| | TGGAAGAACGTGTTGATAAAGGCGAAACCATTCCGCCTCA | |
| | AGAACTGACCACCAAATATGCAGTTGATCCGACCGCAGCA | |
| | GGCACCCTGCAGACCTGGCTGAAAAAAGAAGGTTTTACCA | |
| | TTACCGGTGTGACTCCGGATCGTACCACCATTTATGCAAG | |
| | CGCACCGGCAAGCCAGGTTGAAGCAAGCCTGGGTGTTCAT | |
| | ACCGTTCGTGTTACCCGTGAAGGCCAGACCTATACCGCAG | |
| | CAAGTGATGTTCCGAGCCTGCCGGAAGATATTGGTGGTGC | |
| | CGTTGTTAATATTGGCGGTCTGCAGCCGTATCGTCAGGCA | |
| | CGTAAACATCTGCGTAGCTATATTCAGACCACACCGGAAG | |
| | CAGATGGTGAAGAACCGGCAATTGCAAATGCACCGCCTTA | |
| | TCTGGTTCCGGAAATTCTGAAAGCATATGATGGTGCACGT | |
| | CTGGGTCTGACCGGTAAAGGTCAAGAAATTGCCATTCTGA | |
| | TTGATACCGTTCCGCTGGATACCGATCTGACCAGCTTTTG | |
| | GACCGCAAATGGTGTTGCAGGTAGCCTGGCACGTATTACC | |
| | AAAATCAATGTTAAAGGTGGTGCACTGCCGACACCGAGCG | |
| | GTGAAGAAACCCTGGATGCAGAATGGGCAAGCACCATTGC | |
| | ACCGGATGCAAATGTTCGTATTTATGCCAGCGGTACACTG | |
| | AGCTTTATTGATCTGGATCGTGCACTGGATCGCATTTATG | |
| | CCGATGCACTGGCACAGCCGAAACTGCGTATTGTGAGCAT | |
| | TAGTCTGGGCCTGAGCGAAGCATATATGGCAAAAGGTGAA | |
| | GTTGATGCAGAAGAAGCACGTTTTGTTCGTTTTGCAGCAC | |
| | TGGGTGTTAATGTTTTTGTTAGCACCGGTGATGCCGGTAG | |
| | CAATCCGGGTCCTGATGGTCATCATGCAAATGGTCCGCTG | |
| | GCAGCAGAATGGATGAGCACCAGTCCGCATGTTGTTGCAG | |
| | TTGGTGGCACCAGCCTGCGTCTGGCAAATAATGGTCAGGT | |
| | TGCAAGCGAAACCGGTTGGACCGGTAGCGGTGGTGGTAAA | |
| | AGCAATTTTCAGCCTCGTCCGGCATGGCAGCAAGGTCATG | |
| | GTGTTCCAGCAGGTAATCAGCGTATGGTGCCGGATGTTGG | |
| | TGCAGCAGCCGATCCGAATGAAGGTGCACTGGTTATTCTG | |
| | AATGGTCAGCGTCTGCAGTATGGCGGTACAAGTTGGAGCG | |
| | CACCGATTTGGGCAGGTCTGTGTGCACTGATTAATGAAGC | |
| | ACGTCAGAACAATCATAAAACTCCGCTGCCGTATCTGAAC | |
| | AGCCTGATTTATCCGATGATTGGTAGCAACTGTTTTCGTG | |
| | ATGAACTGACCGGTTCAAATGGTGCATATAGCTGTGGTCC | |
| | GGGTTATGATCTGGTTACCGGTATTGGTAGTCCGGATCTG | |
| | AAACAGCTGGCAGCCAAACTGGCA | |
| 15 | ATGGCAGGCGTTAATGAACCGTATAATGCACGTGAAGATG | Protease 5 |
| | GTATTCCGCTGAAAAGCAGCGCACGTGCAGTTGTTCCGGG | |
| | TGTTAAACTGCATGGTCCGACCGATGGTGCAAGCCGTCTG | |
| | GAAATTACCGTTGTTCTGCGTCGTCGTACCGAACTGCCGA | |
| | GCGCAGCAGCAGATGGTCATCTGACCGCAGCAGAACTGGC | |
| | AAGCGAATATGGTGCATCAGATGATGATGTTCGTCTGGCA | |
| | ACCGAAGTTTTTACCCGTCTGGGTGCAGATGTTGTTGAAA | |
| | GCGATCCGGCAAGTCGTCGTCTGCGTCTGAGCGGCACCGT | |
| | TGAACAGCTGAGCAGCATTTTTGGTACAACCCTGGAAGAT | |
| | GCAACCAGCACCGCACCGGATGGTGCCACCGTTCATTATC | |
| | GTCATCGTCTGGGCGAACTGCGTATTCCGGCAGAACTGAA | |
| | TGGTATTGTTATTGCAGTTCTGGGCTTAGATGATCGTCCG | |
| | CAGGCACGCGCACATTTTCGTATGCTGCCTCGTACCACCG | |
| | CAGGTACAAGCTATAGTCCGGTTGAACTGGGTCGTGTTTA | |
| | TGGTTTTCCGGATGGCACCGATGGTAGCGGTCAGACCGTT | |
| | GCAATTATTGAATTAGGTGGTGGTTATGCACAGGCAGATC | |
| | TGGATGCATATTTTGCAGGTTTAGGTCTGGCCACACCGCA | |
| | GATTACCAGCATTGGTGTTGATGGTGGTGCAAATCAAGGT | |
| | GGTAATGATCCGCAGGGTGCCGATGGTGAAGTGCTGCTGG | |
| | ATATTGAAGTTGTTGGTGCACTGGCACCGAAAGCAGCAAT | |
| | TCAGGTTTATTTTGCACCGAATACCGATGCAGGTTTTCTG | |
| | GATGCCGTTGTTGCAGCAACCAAAGCAGCACCGTGTGCCA | |
| | TTAGCATTAGCTGGGGTCAGAGCGAAGATCAGTGGACCGC | |
| | ACAGGCACGTGATGCATTTGATCAGGCACTGGCAGATGCA | |
| | GCAGCCCTGGGTATTACCACCACCGTTGCAGCCGGTGATC | |
| | GTGGTAGCAGTGATGGTGCCGCAGATGGTAAAGCACATGT | |
| | TGATTTTCCGGCAAGCAGTCCGCATGCACTGGCCTGTGGT | |
| | GGTACACGTCTGGAAGCAGATCCTGCAACCGGTGCAATTC | |
| | GTAGCGAAACCGTTTGGAATGAAGGTCCGGATAGCGCGAC | |
| | CGGTGGTGGCTATAGCAAAGTTTTTCCGCGTCCGAGCTGG | |
| | CAGAGCCCGAGTGCAGGTAAAAGCGGTCGTGGTGTGCCGG | |
| | ATGTTAGCGCAGTTGCAGATCCGCAGACAGGTTATCGTAT | |
| | TCGTGTGGATGGCAAAGATATGGTTATTGGTGGCACCTCA | |
| | GCAGTTGCACCGCTGTGGGCAGCACTGATTGCACGTTTTG | |
| | CCCAGGCAGGTAATCGTCGTTTTGGTCTGATTCAGCCGAG | |
| | CCTGTATGCAGTTAGCAGCGGTTTTCGTGATGTTACCGTT | |
| | GGTGATAATGGTAGCTATCATGCAGGTCCTGGTTGGGATG | |
| | CATGTACCGGTCTGGGCACCCCTGATGGTGCAGCCCTGCT | |
| | GGCAGCCCTGAAAGGT | |
| 16 | ATGAGCACCCGTGCAGCACGTACCACACCGAGCGCACTGG | Protease 6 |
| | CCGATCTGCGTAATGAACCGCGTAGTCCGCTGCCTGGTAG | |
| | CGAAAAAGCAGCACTGGCAGATACACCGGCAACCACCGCA | |
| | GCAGGTATCAAACCGCTGCGTGCAACCGCAGTTGCAAAAG | |
| | CAAAACCGGCAAGCAGCCGCAAAAAAATCACCGTTAGCGT | |
| | TGTTGTTCCGCGTACCAAACCGGTTACACAGGCAGCAGTT | |
| | GCAGGTAAACATCTGACCCGTGCACAGTTTAAAAGCAGCC | |
| | ATGCAGCAGCACCGGCAAGTGTTAAAGCAGTTCAGAAATT | |
| | TGCCAAAGCCTTTAACCTGGTTAGCAAAGCCGAACCGGCA | |
| | CGTAGCACCGTTCATCTGACCGGCACCGTTAAAGATATGC | |
| | AGGATGCATTTGGTGTTACCCTGCAAGAACATACCGTTGG | |
| | TGCAAAAACCCTGCGTATTCGTCAGGGTGCAATTTATCTG | |
| | CCGGATAGCGTTCTGCCGCATGTTCAGGCAGTTCTGGGTT | |
| | TAGATAATCGTCCGCAGGCAAAACCGCATTATCGTGTTGG | |
| | TAAAGCACGTGCAGCAGCCAGCACCAGCTTTACCCCTCCG | |
| | CAGCTGGCACAGCTGTATGGTTTTCCGACCAGCGCAAAAG | |
| | CCACCGGTCAGACCATTGCACTGATTGAATTAGGTGGTGG | |
| | TTTTCGTCAGGCAGATATTACCGCATATTTCAAAAGCCTG | |
| | GGTATTGCAGCACCGAGCGTTAAAGCCGTTCTGGTTGATG | |
| | GTGGTAAAAATGCACCGAGCAATGCAAATGGTGCAGATGG | |
| | TGAAGTTATGCTGGATATTGAAGTTGCAGCGGCAGTTGCA | |
| | CCGGGTGCCAATATTGCAGTTTATTTTGCACCGAATACCG | |
| | ATCAGGGTTTTGTTGATGCAATTGCGACCGCAGCACATGA | |
| | TACCACCAATAAACCGACCATTATTAGCATTAGCTGGGGT | |
| | GGTCCGGAAAGCAGCTGGACCAGCCAGGCACTGACCGCAC | |
| | TGGATAATGCATGTAAAGATGCAGCCGCACTGGGTATTAC | |
| | CGTTACAGCAGCAGCCGGTGATGATGGTTCAGATGATGGT | |
| | GTTGGTGATGGTAAAAAACATGTTGATTTTCCGGCAAGCT | |
| | CACCGAATGTTCTGGCATGTGGTGGCACCAAACTGGTTGC | |
| | AAGCAATGGTGCAATTACCAGCGAAGTTGTTTGGAATGAA | |
| | ACCGCCAATAAAGAAGGTGCAACAGGCGGAGGTATTAGCA | |
| | CCGCATTTCCGCAGCCGACCTGGCAGAAAAGCATTGCAGC | |
| | AACCAAAAGCGGTCGTGGTGTTCCGGATGTTGCGGGTGAT | |
| | GCAGATCCGACCACCGGTTATCAGGTTCGTGTTGATGGTC | |
| | AGAATATGGTTATCGGTGGTACAAGCGCAGTTGCTCCGCT | |
| | GTGGGCAGGTCTGATTGCCCTGAGCAATGCCACCAACAAA | |
| | AATGCAGCAGGTCTGCCGCAGGCCAAACTGTATAGCACCA | |
| | CAGGTCAGAAAGCATTTCGTGATATTACCAGTGGCAATAA | |
| | CGGTGCGTTTAAAGCAGCAAAAGGTTGGGATCCGTGTACC | |
| | GGTCTGGGTAGCCCGAAAGCCGCAAGCATTATTACCCTGC | |
| | TGGCCACCAAAAGCAGCGCCAAAAAGAAAACCAGCCGTGC | |
| | AAAAGCC | |
| 17 | ATGGAAAGCATTATGCCGAGCCAGCCGAGCAGCATTCCGG | Protease 7 |
| | TTCGTGGTAGCGAACGTGCAGCACTGCCGACCGCACATGT | |
| | TGTTGGTCCGGCAGCAAGTGATGAACGTCTGGAAGTTACC | |
| | CTGCGTGTTCGTCCGCGTGCACAGCTGCATGCAAGCGCAA | |
| | GCGAAGCACAGAGCCTGCGTCCGCCTGGTGAACGTAGCTA | |
| | TCTGAGCCGTGAACAGCTGGCAAGCGCACATGGTGCAGCA | |
| | CCGGAAGATATTGCAAAAGTTGAAGCATTTGCCCAGAGCC | |
| | ATGGTCTGCAGGTTGTTCTGACCAGCGCAGCACGTCGTTG | |
| | TGTTATTGTTAGCGGCACCGTTGCAGCCCTGGAAAGCGCA | |
| | TTTGCCGTTAAACTGCAGCAGTATCGTTTTGATGGTGGTA | |
| | GCTATCGTGGTCGTGTTGGTCCTGTTTTTGTTAGTCCGGA | |
| | AATTGGTGATATTGTGGAAGGTGTTTTTGGTCTGGATGAT | |
| | CGTCCGCAGGCAATTGCACATTTTAAACGTAGTGCACATG | |
| | CAGTTCGTGCCGAAGATGGTGCCGCTCCGCATGCCGGTGG | |
| | TGCAAGCTTTACCCCTCCGCAGCTGGCCAAACTGTATAAC | |
| | TATCCGGGTGATACCGATGGCACCGGTCAGTGTATTGGTA | |
| | TTGTTGAATTTGGTGGTGCCATTCGTGCAGCAGATATTCG | |
| | TGCATATTTCAAAGAACTGGGTCTGCCTGCACCGCATGTT | |
| | AATACCGTTCTGGTTGATCATGCACATATGCGTAGTGATG | |
| | ATGCAGATGCAGAAGTTATGCTGGATATTGAAGTTGCAGC | |
| | AGCCATTGCACCGAAAGCACAGATTGTTGTTTATTTTGCT | |
| | CCGAATACCAGCCAGGGTTTTATTGATGCATTTACCCATG | |
| | CAATTCATGACACCGTTCATAAACCGAGCGTTATTAGCGT | |
| | TAGCTGGGGTGGTCCGGAAAAAGATTGGAGCGCACAGATT | |
| | AAAACCCAGCTGGATCAGGTTTTTCAGGATGCAGCAGCAC | |
| | TGGGTGTTACCATTTGTGCAGCCAGCGGTGATGCAGGTAG | |
| | CAGTGATGAAAATCCGGATGCACTGGCAAGCATTGGTCTG | |
| | ACACCGGATGGTCTGAGCCATGCAGATTTTCCGGCAAGCA | |
| | GCCCGTTTGCACTGGCCTGTGGTGGCACCAAACTGGTTGC | |
| | CAGCGCCAGCGCAATTACCAGCGAAACCGTTTGGAATGAA | |
| | GATCCGGTGCGTAGCGCAACCGGTGGTGGTATTAGCGATT | |
| | TTTTTGATGTTCCGGGTTATCAGGCAACCGCAAATATTCC | |
| | GGTTAGCGCAAATCCTGGTGGTCGTAAAGGTCGTGGTGTT | |
| | CCGGATATTGCAGCCGATGCAGATCCGGCAACCGGTTATC | |
| | TGGTTCGTGTTCATGGTCAGGATGCCGTTATTGGTGGTAC | |
| | AAGCGCAGTTGCACCGCTGATGGCAGGTCTGGTTGCACTG | |
| | CTGAATCATAAACTGGGTCATCCGGTTGGTTTTCTGAATC | |
| | CGCTGCTGTATCGTACCGCAGGTATTACCCGTGATATTAC | |
| | CCAGGGCAATAATGGTGCATATGCCGCAGGTAAAGGTTGG | |
| | GATGCATGTACCGGTCTGGGTGTGCCGGATGGTGCCAAAC | |
| | TGCTGGATGCCCTGATG | |
| 18 | ATGCCGCAGAGCCAGAATCGTGTTGTTGTTCGTGGTAGCG | Protease 8 |
| | AACGTCAGCCGATGCCGAAAGCACATAGCCAGCATGCACT | |
| | GCCTCCGACCGAACGTCTGGAAGTTACCGTTCGTCTGCGT | |
| | CCGAAAGCAGCACTGGCAAGCGCAGCAGCAAGCAGCCATG | |
| | CAATGGCAGATGTTCCGCCTAGCCAGCGTACCTATCTGAG | |
| | CCGTGAAGAACTGGCAGCACAGTGTGGTGCAAGCGAAGAT | |
| | GATGCACAGGCAGTTGCAGATTTTGCACATGCACATGGTC | |
| | TGGTTGTTATTCATACCGATCTGGCACGTCGTAGCGTTCT | |
| | GCTGGCAGGCACCGCAGCCGATTTTGGTGCAGCATTTGGC | |
| | ACCCAGCTGCATCAGTATAGCAGTCCGGAAGGCACCTATC | |
| | GTGGTCGTACCGGCACCGTGACCGTTCCGGCACCGCTGGC | |
| | AGATATTGTTCAGGGTGTTTTTGGTCTGGATGATCGTCGT | |
| | CAGGCAGAACCGCATTTTCAGGTTCGTCCGGGTCCGACAC | |
| | CGGCTCCGGGTGCAATTGTAGCACGTGCAGCCGGTCAGAG | |
| | CTTTACCCCTCCGCAGCTGGCACAGCTGTATGATTTTCCA | |
| | GGTGGCCTGGATGGCACCGGTCAGACCATTGCAGTTATTG | |
| | AATTAGGTGGTGGTTTTAAACCGGCAGATCTGAAAGCATA | |
| | TTTTACCGGTCTGAACCTGCCGGTGCCGACCGTTAAAGTT | |
| | GTTAGCGTTAATGGTGGTCGTAATCAGCCGACCAATGCAA | |
| | ATAGCGCAGATGGTGAAGTGCTGCTGGATATTGAAGTTGC | |
| | AGCAGCAGTTGCACCGCGTGCACATCTGGTGGTTTATTTT | |
| | GCACCGAATACCAGCCAGGGTTTTCTGAATGCAATTACCA | |
| | CCGCAGTTCATGATAAAGTGAATAATCCGGGTATTATCAG | |
| | CATTAGCTGGGGTGGTCCGGAAAGTACCTGGACCGGTCAG | |
| | GCAATGGATCAGTTTGATCAGGCATTTCAAGAGGCAGCAA | |
| | TGCTGGGTGTTACCGTTTGTGTTGCAGCGGGTGATAATGG | |
| | TAGTGCCGATGGTGTTGCAGATGGTCAGCCTCATGCCGAT | |
| | TTTCCGGCAAGCAGTCCGTTTGCACTGGCATGTGGTGGCA | |
| | CCAAACTGACCGCAAGCGGTCCGACCATTAGTAGCGAAGT | |
| | TGTTTGGAATGAAGGTCCGAATAGCGCAACCGGTGGTGGT | |
| | CTGAGCGCACATTTTCCGGTTCCGGCATATCAGCAGCAGC | |
| | TGAAATTTCCGACACCTCCGGCAGGCGCAAAAGCAGGTCG | |
| | TGGTCTGCCGGATGTTGCCGGTGATGCAGATCCGAATACC | |
| | GGTTATCAGGTGCGTGTTGATGGTCAGAATCTGGTTATTG | |
| | GTGGTACAAGCGCAGTTGCTCCGCTGTGGGCAGGTCTGCT | |
| | GGCCCTGCTGAATCAGAAACTGCCGAAACCGGTTGGCTTT | |
| | CTGAATCCGCTGCTGTATGGTAGCCTGGCAGGTCAGGGTG | |
| | TGACCCGTGATATTACCTCTGGTAATAATGGTGCATTTGC | |
| | AGCAGGTCCTGGTTGGGATGCATGTACCGGTTGGGGTAGT | |
| | CCGGTTGGTGGTAAACTGTTAGCAGCCCTGCAAGGTGGTG | |
| | CAGCCGTTGCA | |
| 19 | ATGAGCAAACATCCGCTGATGGGTAGCGAACGTGCACCGT | Protease 9 |
| | TTGATGGTGCACAGAGCGTTGGTAAAGCAGATCCGGCAGA | |
| | ACGTCTGGAAGTTACCGTTCTGGTTCGTCGTGGTAGCAGT | |
| | GATGCCCTGCGTACCCGTGTTAGCAAACTGGTTGCAGGTA | |
| | ATGCAAGTGATGGTCATATTCAGCGTGAAGATTTTGCACA | |
| | GCAGTTTGGTGCAGCACCGAATGATATGAGCGCAGTTCGT | |
| | AATTTTGCAAGCCAGCATGGTCTGAGCGTTGTTGAAGAAC | |
| | ATGCAGCACGTCGTACCGTTATTCTGAGCGGCACCGTTGC | |
| | ACAGTTTAATGATGCATTTGATGTTGATCTGCAGCAGTTC | |
| | GAACATGCCGGTGGTAGCTATCGTGGTCGTACCGGTCCGG | |
| | TTCATCTGCCTGATGAACTGAGCGGTGTTGTTGATGCAGT | |
| | TCTGGGTTTAGATAATCGTCCGCAGGCACGTCCGCATTTT | |
| | CGTAGCCGTCCGCCTCAGGGTAATGTTCATTGGCAGAGCA | |
| | GCCGCACCGGTACAACCAGCAGCACACCGCTGCAGCTGGC | |
| | AAGCCTGTATGATTTTCCGGCAGGCACCGGTCAGGGTCAG | |
| | TGTATTGCAATTATTGAATTAGGTGGTGGTTATCGTCCGG | |
| | CAGATCTGAAAGCATATTTTAGTAAACTGGGTATTGCGAG | |
| | CCCGAAAGTTACCACCGTTAGCGTTGATCATGGTAAAAAT | |
| | CATCCGACCGGTGATGCAAATGGTCCGGATGGTGAAGTTA | |
| | TGCTGGATATTGAAATTGCCGGTGCAATTGCCCCTGGTGC | |
| | ACATATTGCAGTTTATTTTGCACCGAATACCGATGCAGGT | |
| | TTTCTGGATGCAGTGACCACCGCAATTCATGATACCATTC | |
| | GTAAACCGAGCGTTATTAGCATTAGCTGGGGTGGTCCGGA | |
| | AAGCGCATGGACCGAACAGGCAATGACCGCATTTGATCAG | |
| | GCATTTCAGGCAGCAGCAGCCCTGGGTATTACCGTTTGTG | |
| | TTGCAAGCGGTGATAATGGTAGTGGTGATGGTGTTAATGA | |
| | TGGTGCCGATCATGTTGATTTTCCAGCAAGCAGCCCGTAT | |
| | GCACTGGCATGTGGTGGCACCAGCGTTCAGGCAGGTAAAG | |
| | GTGCCATTGCAAAAGAAACCGTTTGGAATGATGGCGCAAA | |
| | TGGTGGTGCCAGCGGTGGTGGTGTTAGCAGCTTTTTTGCA | |
| | CTGCCTGCATGGCAAGAAGGTCTGCAGGCAGCACGTGCAA | |
| | AAGGTGGTACAGGTGCACTGCAGATGCGTGGTGTTCCGGA | |
| | TGTGGCAGGCAATGCCGATCCGGCAACCGGTTATGATGTT | |
| | CGTGTTGATGGTAGCGATATGGTTATTGGTGGTACAAGCG | |
| | CAGTTGCACCGCTGTGGGCAGGTCTGGTTGCACGTATTAA | |
| | TGCAGGTAAAAATAGTCCGGCAGGTTATCTGAATCCGAAA | |
| | CTGTATAAAACCGCAGCAGGTCTGACCGATATCACCCAGG | |
| | GTAATAATGGTGATTTTGTTGCCAGCGCAGGTTGGGATGC | |
| | ATGTACCGGTCTGGGTCGTCCTGATGGTAATAAACTGGCA | |
| | GGTACATTTGGT | |
| 20 | ATGGACGAAACCAATTTTACCAGCACCAATGGTAGTCCGC | Protease 10 |
| | AGTATATTCCGGTTACCGGTAGCGCACGTGCAATTGTTCC | |
| | GGGTGCAACCCATGCAGGTCATACCGATGATAATGAAGTT | |
| | CTGAGCGTTACCCTGCAGCTGCGTCGTCCGAGCGCAGATG | |
| | AACTGACCGCACATGTTGAAGCACTGGGTACAACCCCTCC | |
| | GGCAAATCGTAAACATATGACCCATGATGAATTTGAAGCA | |
| | AGCCATGGTGCAAGTGATGATGATCTGAATCTGGTTACCG | |
| | CATTTGCAACCGAACAGGGTCTGAGCGTGGAACGTATTAA | |
| | CAAAGCAGCAGCAACCGTTCATGTTAGCGGTACAGCCGGT | |
| | GCATTCAATAAAGCATTTCATGTTCAGCTGGGCAATTATC | |
| | AGCATCCGGATTTTACCTATCGTGGTTATGATGGTCCGGT | |
| | TCATATTCCGGCACATCTGACCGATATTGTTACCGGTGTT | |
| | CTGGGTTTAGATAATCGTCCGCAGGCAAAACCGCATTTTC | |
| | GTGTTTATCAAGAAGCAGCAGTTCGTAGCAATGCACTGGC | |
| | AGCACCGATTAGCTATACCCCGACACAGGTTGCAGCACTG | |
| | TATAACTTTCCGACCAATGTTGATTGTAGCGGTCAGTGTA | |
| | TTGGCATTATTGAATTAGGTGGTGGCTACAGCAAAAGCAA | |
| | TCTGGATCAGTATTTTGCAAGCCTGGGTGTTCCGACACCG | |
| | ACCATTACCAGCGTTAGCGTTGATGGTGGTCAGAATCAGC | |
| | CGACCGGTAGTCCGAATGGTCCGGATGGTGAAGTTGATCT | |
| | GGATATTGAAGTTGCAGCAAGCGTTGCACCGGGTGCACAT | |
| | ATTGCAGTTTATTTTGCACCGAATACCGATGCAGGTTTTC | |
| | TGGATGCAATTACCACCGCAGTTCATGACAAAACCAATAA | |
| | ACCGAGCGTTATTAGCATTAGCTGGGGTGGTCCGGAAATG | |
| | AGCTGGACCACACAGGCAATGCAGGCCATGAATAATGCAA | |
| | TGCAGAGCGCAGCCGCACTGGGTGTTACCATTACCGTTGC | |
| | AGCCGGTGATAATGGTAGCACCGATGGTGTTAATGATGGT | |
| | AGCTTTCATGTTGATTTTCCGGCAAGCGCACCGTATGCGC | |
| | TGGCATGTGGTGGCACCCATCTGGTTGGTAGCGGTAGCAC | |
| | CATTGAAAGCGAAACCGTTTGGAATGATGGTGCAAATGGT | |
| | GGTGCGACCGGTGGTGGTGTTAGCAGCGTTTTTCCGGTTC | |
| | CGAGCTGGCAGCAGAAAGCAAATGTTCCGCCTAGCGCAAA | |
| | TCCTGGTGCAGGCACCGGTCGTGGCGTTCCGGATGTGAGC | |
| | GGTGATGCAGATCCGGCAACCGGTTATCAGGTTCTGGTTG | |
| | ATGGTCAGCAGTTTCCGATTGGTGGTACAAGCGCAGTTGC | |
| | ACCGCTGTGGGCAGGTCTGGTTGCACTGGCCAATCAGACG | |
| | CTGGGTAAACCGGTTGGTTATATCAATCCGCTGCTGTATA | |
| | GCATTCCTGCACAGGATAATGCCTTTCATGATATTACCCA | |
| | GGGCAATAATGATCCGAATCAGACCGGTCAGGTTTATCCG | |
| | GCAGGTCCAGGTTGGGATGCATGTACCGGTCTGGGTTCAC | |
| | CGAATGGCACCCTGCTGATTCAGGCACTGGGTCAGATTGG | |
| | T | |

What is claimed is:

1. A foodstuff comprising a recombinant S53 family protease and a legume protein.

2. The foodstuff of claim 1, wherein the S53 family protease comprises an amino acid sequence having at least 90% sequence identity to SEQ ID NO: 1.

3. The foodstuff of claim 1, wherein the S53 family protease comprises the amino acid sequence SEQ ID NO: 1.

4. The foodstuff of claim 1, wherein the S53 family protease comprises an active site comprising one or more amino acid substitutions of residues E266, F295, S316, W317, G318, A349, A350, G351, D352, S353, D367, G462, G463, T464, S465, and A466 with respect to SEQ ID NO:1.

5. The method of claim 4, wherein the active site comprises from two to five amino acid substitutions.

6. The foodstuff of claim 4, wherein the S53 family protease comprises an active site comprising amino acid residues E266, F295, S316, W317, G318, A349, A350, G351, D352, S353, D367, G462, G463, T464, S465, and A466 with respect to SEQ ID NO:1.

7. The foodstuff of claim 1, wherein the S53 family protease comprises one or more truncations of SEQ ID NO:1, wherein the one or more truncations comprises an N-terminal truncation, a C-terminal truncation, or both an N-terminal and a C-terminal truncation.

8. The foodstuff of claim 1, wherein the S53 family protease is active at a pH range from about pH 2 to about pH 5.

9. The foodstuff of claim 1, further comprising a carbohydrate.

10. The foodstuff of claim 1, further comprising a sugar.

11. The foodstuff of claim 10, wherein the sugar is selected from sucrose, lactose, maltose, and another di-, tri-, or polysaccharide which comprises glucose as a monomer.

12. The foodstuff of claim 11, wherein the sugar comprises sucrose.

13. The foodstuff of claim 1, further comprising a fruit.

14. The foodstuff of claim 1, wherein the legume protein is provided in solid form.

15. The foodstuff of claim 1, wherein the legume protein is provided in liquid form.

16. The foodstuff of claim 15, wherein the liquid form is a drink.

17. The foodstuff of claim 16, wherein the drink is a protein shake.

18. The foodstuff of claim 17, further comprising other commonly used ingredients found in protein shakes.

19. The foodstuff of claim 1, wherein the legume protein is a pea protein.

20. The foodstuff of claim 1, wherein the foodstuff comprises a protein concentrate comprising protein material that is obtained from a legume upon removal of a soluble carbohydrate, ash, and/or other minor constituents.

21. The foodstuff of claim 1, wherein the foodstuff comprises a soluble carbohydrate, ash, and/or other minor constituents obtained from a legume.

22. The foodstuff of claim 1, wherein the foodstuff is suitable for consumption by a mammal.

23. The foodstuff of claim 22, wherein the mammal is a human.

24. The foodstuff of claim 23, wherein the human has hyperglycemia.

25. The foodstuff of claim 1, wherein the foodstuff reduces an increase in blood glucose in a subject upon consumption of the foodstuff.

26. The foodstuff of claim 25, wherein the increase in blood sugar is reduced relative to consumption of an equivalent foodstuff lacking the S53 family protease.


SEQUENCE LISTING (46 sequences)

SEQ ID NO: 1 - DNA, 1586 bp, synthetic construct
  misc_feature 1..1586: note = Description of Artificial Sequence: Synthetic polynucleotide
  source 1..1586: mol_type = other DNA
gaaataattttgtttaactttaagaaggagatatacatatgagcgaacctgttccggcagcagcacgtcgtaccattccgggtagcgaacgtccgcctgttgataccgcagcagcagcccgtcaggcagttcctgcagatacccgtgttgaagcaaccgttgttctgcgtcgtcgtgcagaactgccggatggtccgggtctgctgacaccggcagaactggcagaacgtcatggtgcagatccggcagatgttgaactggttacccgtacactgaccggtctgggtgttgaagttaccgcagttgatgcagcaagccgtcgtctgcgtgttgccggtccggcaggcgttctggcagaagcatttggcaccagcctggcacaggttagcacaccggatccgagcggtgcccaggttacccatcgttatcgtgccggtgcactgagcgttccagccgaactggatggtgttgtgaccgcagttctgggtttagatgatcgtccgcaggcacgtgcgcgttttcgtgttgcaacggcagccgcagcaagcgcaggttataccccgattgaactgggtcgtgtttatagctttccggaaggtagtgatggtagcggtcagaccattgcaattattgaattaggtggtggttttgcacagagtgaactggatacctattttgcaggtctgggtattagcggtccgaccgttacagcagttggtgttgatggtggtagcaatgttgcaggtcgtgatccgcagggtgcagatggtgaagttctgctggatattgaagttgcgggtgcactggcaccgggtgccgatgttgttgtttattttgcaccgaataccgatgcaggttttctggatgcagttgcacaggcagcacatgcaaccccgactccggcagccattagcattagctggggtggtagcgaagatacctggacaggtcaggcacgtaccgcctttgatgcggcactggcagatgcagccgcactgggtgttaccaccaccgttgcagccggtgatgatggtagtaccgatcgtgcaaccgatggtaaaagccatgttgattttccggcaagcagtccgcatgcactggcctgtggtggcacccatctggatgccaatgcaaccaccggtgcagttaccagcgaagttgtttggaataatggtgcaggtaaaggtgcaaccggtggcggtgttagcaccgtttttgcccagccgagctggcaggcaagtgccggtgttccggatggccctggtggtaaacctggtcgtggtgtgccggatgttagcgcagttgccgatccgcagaccggttatcgtattcgtgtggatggtcaggatctggttattggtggtacaagcgcagtggcaccgctgtgggcagcactggttgcacgtctggttcaggcaggtcgcgcaaaactgggcctgctgcagccgaaactgtatgcagcaccgaccgcatttcgtgatattaccgaaggtgataatggcgcatatcgtgcaggtcctggttgggatgcatgtacaggcctgggcgttccggttggcaccgcactggcgagcgcactgagttga

SEQ ID NO: 2 - protein, 515 aa, Pseudonocardia sp.
MSEPVPAAARRTIPGSERPPVDTAAAARQAVPADTRVEATVVLRRRAELPDGPGLLTPAELAERHGADPADVELVTRTLTGLGVEVTAVDAASRRLRVAGPAGVLAEAFGTSLAQVSTPDPSGAQVTHRYRAGALSVPAELDGVVTAVLGLDDRPQARARFRVATAAAASAGYTPIELGRVYSFPEGSDGSGQTIAIIELGGGFAQSELDTYFAGLGISGPTVTAVGVDGGSNVAGRDPQGADGEVLLDIEVAGALAPGADVVVYFAPNTDAGFLDAVAQAAHATPTPAAISISWGGSEDTWTGQARTAFDAALADAAALGVTTTVAAGDDGSTDRATDGKSHVDFPASSPHALACGGTHLDANATTGAVTSEVVWNNGAGKGATGGGVSTVFAQPSWQASAGVPDGPGGKPGRGVPDVSAVADPQTGYRIRVDGQDLVIGGTSAVAPLWAALVARLVQAGRAKLGLLQPKLYAAPTAFRDITEGDNGAYRAGPGWDACTGLGVPVGTALASALS

SEQ ID NO: 3 - DNA, 1634 bp, synthetic construct
  misc_feature 1..1634: note = Description of Artificial Sequence: Synthetic polynucleotide
  source 1..1634: mol_type = other DNA
gaaataattttgtttaactttaagaaggagatatacatatggccgatgatagcagcccgaccaccgcagcagatcgtccgacactgcctggtagcgcacgtcgtccggttgcagcagcacaggcagcaggtccgctggatgatgcagcaccgctggaagttaccctggttctgcgtcgtcgtaccgcactgccagcaggcacaggtcgtccggcaccgatgggtcgtgcagaatttgcagaaacccatggtgcagatccggcagatgccgaaaccgttaccgcagcactgaccgcagaaggtctgcgtattaccgcagttgatctgccgagccgtcgtgttcaggttgccggtgatgttgcaacctttagccgtgtttttggtgttagcctgagccgtgttgaaagccctgatccggttgccgatcgtctggttccgcatcgtcagcgtagcggtgatctggcagttcctgctccgctggcaggcgttgtgaccgcagttctgggtttagatgatcgtccgcaggcacgtgcactgtttcgtcctgcagcagccgttgataccacctttactccgctggaactgggtcgtgtttatcgttttccgagcggtacagatggtcgtggtcagcgtctggcaattctggaattaggtggtggttatacccaggcagatctggatgcatattggaccaccattggtctggcagatccgcctaccgttacagcagttggtgttgatggtgcagcaaatgcaccggaaggtgatccgaatggtgccgatggtgaagttctgctggatattgaagttgcgggtgcactggcaccgggtgccgatctggttgtttattttgcaccgaataccgatcgtggttttctggatgccctgagcaccgcagtgcatgccgatccgacaccgaccgcagtgagcattagctggggtcagaatgaagatgaatggaccgcacaggcacgtaccgcaatggatgaagcactggcagatgcagccgcactgggtgttaccgtttgtgcagcagcgggtgatgatggtagcacagataacgcaccggatggtcaggcacatgttgattttccggcaagcagtccgcatgcgctggcatgtggtggtacaaccctgcgtgcggatccggataccggtgaagttagcagcgaaaccgtgtggtttcatggcaccggtcaaggtggtactggtggtggtgtgagcgcagtttttgcagttccggattggcaggatggtgttcgtgttccgggtgatgcagataccggtcgtcatggtcgcggtgttccggatgttagcgcagatgctgatccgagtaccggttatcaggttcgtgtggatggtacggatgcagtgtttggtggcaccagcgcagttagtccgctgtggtctgcactgacctgtcgtctggccgaagcgctgggacagcgtccgggtctgctgcagccgctgatttatgcaggtctgagcgcaggcgaagttgcagccggttttcgtgatgttaccagcggtagcaatggtgcatacgatgcaggtcctggttgggatccgtgcaccggtctgggtgtgccggatggcgaagcactgctggttcgtctgcgtacagcactgggctga

SEQ ID NO: 4 - protein, 531 aa, Modestobacter sp.
MADDSSPTTAADRPTLPGSARRPVAAAQAAGPLDDAAPLEVTLVLRRRTALPAGTGRPAPMGRAEFAETHGADPADAETVTAALTAEGLRITAVDLPSRRVQVAGDVATFSRVFGVSLSRVESPDPVADRLVPHRQRSGDLAVPAPLAGVVTAVLGLDDRPQARALFRPAAAVDTTFTPLELGRVYRFPSGTDGRGQRLAILELGGGYTQADLDAYWTTIGLADPPTVTAVGVDGAANAPEGDPNGADGEVLLDIEVAGALAPGADLVVYFAPNTDRGFLDALSTAVHADPTPTAVSISWGQNEDEWTAQARTAMDEALADAAALGVTVCAAAGDDGSTDNAPDGQAHVDFPASSPHALACGGTTLRADPDTGEVSSETVWFHGTGQGGTGGGVSAVFAVPDWQDGVRVPGDADTGRHGRGVPDVSADADPSTGYQVRVDGTDAVFGGTSAVSPLWSALTCRLAEALGQRPGLLQPLIYAGLSAGEVAAGFRDVTSGSNGAYDAGPGWDPCTGLGVPDGEALLVRLRTALG

SEQ ID NO: 5 - DNA, 1613 bp, synthetic construct
  misc_feature 1..1613: note = Description of Artificial Sequence: Synthetic polynucleotide
  source 1..1613: mol_type = other DNA
gaaataattttgtttaactttaagaaggagatatacatatggattatcagattctgcgtggtagcgaacgtagtccgctgcctggttgtaccgataccggtaaatttccggcagcacatcgtctgcgtgttctgctggcactgcgtcagccggaactggatgcagcagcagcccgtctgctggatacagccggtgatgaactgcctgcaccgctgagccgtgatgcatttgcaacccgttttgcagcagccgcagatgacctgcgtgcagttgaagcatttgcgacccagcatggtctgagcatggaacagaccctggcacatgccggtgttgcaattctggaaggtagcgttcagcagtttgatcgtgcatttcaggttgatctgcgtgattatcgtaaagatgatctgcgctatcgtggtcgtaccggtgcagttagcattccgaccgcactgcatggtgttgttagcgcagttctgggtttagatgatcgtccgcaggcacataccctgccgcaggcgcaggatgcaccagcaccagctggcgcagcagcaccgattgcacgttatacccctccgcagctggcagaactgtatggttttccggaacatgatggtgcaggtcagtgtattggtattattgcattaggtggtggttatgaacgtgcacaactggcagcatattttaccgaactgggtctgccgatgccgcagattgttgatgtactgctggcaggcgcacgtaatcagcctggtggtcagggtcgtaaagcagatattgaagttcagatggatgttcagattgccggtgcaattgcccctggtgccaaactggttgtttattttgcaccgaataccgataatggctttctggaagcaattgtgagcgcaattcatgatcgtgcccatgcaccggatgttattgcaatttcatggggttttacagaaaccctgtggaccgcacagagccgtgcagcatataatcgtgcactgcaggcagcagcgctgatgggtattaccgtttgtattgcaagcggtgatgatggcgcaagtgatggtcagccaggtctgaatgtttgttttccggcaagcagtccgtttgttctggcatgtggtggcacccgtctgcaggttgatgttcaggcacagcatgaacaggcatggtcaggcaccggtggtggccagagtcgtgtttttgcacgtccgcgttggcagcaggcactgacgctgcatggcacccagcagacagcacagccgctgagcatgcgtggtgttccggatgttgcagcaaatgcagatgcagaaaccggttattatgtgcatattgatggtcgtccggcagttatgggtggcaccagtgcagccgcaccggtttgggcagcactgttagcacgtgtttatggcctgaatggtggtcgtcgtgtgtttctgcctccgcgtctgtatgcagttgcagatgtttgtcgtgatattgtggatggtggtaatggtggttttgttgcaagccctggttgggatgcatgtaccggtctgggtgtgccggatggtggccgtattgccgcagccttaggtgccggtccgggtgcaaaaccggcaattaccccgacaggctga

SEQ ID NO: 6 - protein, 524 aa, Xanthomonas translucens
MDYQILRGSERSPLPGCTDTGKFPAAHRLRVLLALRQPELDAAAARLLDTAGDELPAPLSRDAFATRFAAAADDLRAVEAFATQHGLSMEQTLAHAGVAILEGSVQQFDRAFQVDLRDYRKDDLRYRGRTGAVSIPTALHGVVSAVLGLDDRPQAHTLPQAQDAPAPAGAAAPIARYTPPQLAELYGFPEHDGAGQCIGIIALGGGYERAQLAAYFTELGLPMPQIVDVLLAGARNQPGGQGRKADIEVQMDVQIAGAIAPGAKLVVYFAPNTDNGFLEAIVSAIHDRAHAPDVIAISWGFTETLWTAQSRAAYNRALQAAALMGITVCIASGDDGASDGQPGLNVCFPASSPFVLACGGTRLQVDVQAQHEQAWSGTGGGQSRVFARPRWQQALTLHGTQQTAQPLSMRGVPDVAANADAETGYYVHIDGRPAVMGGTSAAAPVWAALLARVYGLNGGRRVFLPPRLYAVADVCRDIVDGGNGGFVASPGWDACTGLGVPDGGRIAAALGAGPGAKPAITPTG

SEQ ID NO: 7 - DNA, 1691 bp, synthetic construct
  misc_feature 1..1691: note = Description of Artificial Sequence: Synthetic polynucleotide
  source 1..1691: mol_type = other DNA
gaaataattttgtttaactttaagaaggagatatacatatgacccgtcatccggttagcgatagcggtgcaagcaatgaacatccggttccggcaggcgcacagtgtatgggtgcatgtgatccggcagaacattttaatgttgttgttattgttcgtcgtcagagcgaacgtgcatttcgtgaactggttgaacgtattgcaacaggtgcaccgggtgcgcagccgattagccgtgaacagtatgaacagcgttttagcgcagatgcagcagatgttgcacgtgttgaagcatttgcaaaaacccatggtctggttgttgtgaaagcagatcgtgatacccgtcgtgttgttctgagcggcaccgttcagcagtataatgcagcatttggtgttgatctgcagcgttttgaacatcaggttggtaaactgaaacagcattttcgtcagccgaccggtccggttcatctgccggaagatctgcatgaagttattaccgcagttgttggtctggatagccgtgcaaaagttcagccgcattttcgcattgatagccagacaccggcaacaccgcctgaaaaagcaagccagcctggtgatggtgttgttcatgcaccgattcgtgcagcacgtgcagttagccgtagctttacaccgctgcagctggcagaactgtatgattttccgccaggtgatggtaaaggtcagtgtattgcactgattgaaatgggtggtggttatgcacagagcgatctggatgcatattttagtgcactgggtgttacccgtccgcgtgtggaagcagttagcgttgatcaggcaaccaatgcaccgagcggtgatccgaatggtccggatgccgaagttaccctggatgttgaaattgccggtgcactggctccgggtgctctgattgcagtttattttgcaccgaatagcgaagccggttttgttgatgccgttagcgcagcactgcatgatagtcagcgtaaagcagcaattattagcattagctggggtgctccggaaagcatttggagccagcagaccctgggtgcactgaatgatgcactgcagaccgcagtggccctgggtgtgaccgtttgttgtgcaagcggtgatagcggtagctcagatggtgttaccgatggtgcagatcatgtggattttccggcaagcagcccgtatgcattaggttgtggtggcacccagctgaccgcagcaaatggtcgtattacccgtgaaaccgtttggggtagcggtgccaatggtgcaaccggtggtggtgttagcgcaacctttgcagttccggcatggcagaaaggtctgaaagtgagccgtggtagtggtgccgcacgtgccctggcactggcacgtcgtggtgttccggatgttgcagccgatgcagatccggcaaccggttatgaagttcatattggtggtatggataccgttgttggtggtacaagcgcagttgctccgctgtgggcagcactggttgcccgtattaatgcaggtagcggtaaagccgcaggttttatcaatgccaaactgtatgcacgtccgggtgcatttaatgatatcaccagcggtagcaatggtgattatgcagcccgtcctggttgggatgcatgtaccggtctgggtacaccggttggtacacgtgttgcagcggcaattggtagcgcatga

SEQ ID NO: 8 - protein, 550 aa, Paraburkholderia sacchari
MTRHPVSDSGASNEHPVPAGAQCMGACDPAEHFNVVVIVRRQSERAFRELVERIATGAPGAQPISREQYEQRFSADAADVARVEAFAKTHGLVVVKADRDTRRVVLSGTVQQYNAAFGVDLQRFEHQVGKLKQHFRQPTGPVHLPEDLHEVITAVVGLDSRAKVQPHFRIDSQTPATPPEKASQPGDGVVHAPIRAARAVSRSFTPLQLAELYDFPPGDGKGQCIALIEMGGGYAQSDLDAYFSALGVTRPRVEAVSVDQATNAPSGDPNGPDAEVTLDVEIAGALAPGALIAVYFAPNSEAGFVDAVSAALHDSQRKAAIISISWGAPESIWSQQTLGALNDALQTAVALGVTVCCASGDSGSSDGVTDGADHVDFPASSPYALGCGGTQLTAANGRITRETVWGSGANGATGGGVSATFAVPAWQKGLKVSRGSGAARALALARRGVPDVAADADPATGYEVHIGGMDTVVGGTSAVAPLWAALVARINAGSGKAAGFINAKLYARPGAFNDITSGSNGDYAARPGWDACTGLGTPVGTRVAAAIGSA

SEQ ID NO: 9 - DNA, 1637 bp, synthetic construct
  misc_feature 1..1637: note = Description of Artificial Sequence: Synthetic polynucleotide
  source 1..1637: mol_type = other DNA
gaaataattttgtttaactttaagaaggagatatacatatggtgcgtcatccgctgcgtggtagcgaacgtaccattccggaagatgcacgtattctgggtgatgcacatccggcagagcagattcgtgcactggttcagctgcgtcgtccgaatgaagcagaactggatgttcgtctgagcggttttgttcatgcacatgcagcaggcaccccgagtccgacaccgctgacacgtgaagaatgggcagcacagtttggtgcagcaaccgatgatattgatgcagttcgtacctttgcacgtgaacatggtctgcaggttgccgaagttaatgttgcagcagccaccgttatgctggaaggtagcgttgaacagttttgtcgtgcatttgatacccatctgcatcgtgttgcacatggtggtagtgaatatcgtggtcgtagcggtccgctgcgcctgccggaaagcctgcaggatgttgttgttgcagttctgggtttagatagccgtccgcaggcagcaccgcattttcgttttgttccgctgccgaccggtagcgtggaacctggtggtattcgtccggcacgtgcagcaccgaccgcaagctatacaccggtgcagctggcacagctgtatggttttccgcaaggtgatggtgcaggtcagtgtattgcatttgttgaattaggtggtggttatcgcgaagatgatctgcgtgcatattttcaagaggttggtatgccgatgccgaccgttaccgcaattccggttggtcagggtgcaaatcgtccgaccggtgatccgagcggtccggatggtgaagtgatgctggatctggaagttgcgggtgcagccgcaccgggtgcaaccctggcagtgtattttaccgttaataccgatgcaggttttgtgcaggcaattaatgcagcaattcatgataccaaactgcgtccgagcgttgttagcattagctggggtgcaccggaaagcgcatggacaccgcaggcaatgcaggccgttaatgccgcactgcagagcgcagcaaccatgggtgttaccgtttgtgcagccagcggtgatagcggtagcagtgatggtcagccggatcgtgttgatcatgttgattttccggcaagcagcccgtatgcactggcatgtggtggcaccagcgttcgtgcaagcggtaatcgtattgccgaagaaaccgtttggaatgatggtgcccgtggtggtgcaggcggtggtggtgttagcaccgtttttgcactgccgagctggcagcaaggtctggcagcccagcagaccggtggtgattcagttccgctggcacgtcgtggtgttccggatgttagcgcagatgcagatccgctgaccggttatgttgttcgcgttgatggtgaaagcggtgttgttggtggtacatcagctgccgcaccgctgtgggcagccctgattgcccgtattaatgcaattaaaggccgtccggcaggttatctgcatgcacgtctgtatcagaatccgggtgcatttaatgatattaagcagggtaataatggtgcctttgccgcagcacctggttgggatgcatgtaccggtctgggtagcccgaaaggtgatgcaattgccaacctgttttga

SEQ ID NO: 10 - protein, 532 aa, Paraburkholderia sacchari
MVRHPLRGSERTIPEDARILGDAHPAEQIRALVQLRRPNEAELDVRLSGFVHAHAAGTPSPTPLTREEWAAQFGAATDDIDAVRTFAREHGLQVAEVNVAAATVMLEGSVEQFCRAFDTHLHRVAHGGSEYRGRSGPLRLPESLQDVVVAVLGLDSRPQAAPHFRFVPLPTGSVEPGGIRPARAAPTASYTPVQLAQLYGFPQGDGAGQCIAFVELGGGYREDDLRAYFQEVGMPMPTVTAIPVGQGANRPTGDPSGPDGEVMLDLEVAGAAAPGATLAVYFTVNTDAGFVQAINAAIHDTKLRPSVVSISWGAPESAWTPQAMQAVNAALQSAATMGVTVCAASGDSGSSDGQPDRVDHVDFPASSPYALACGGTSVRASGNRIAEETVWNDGARGGAGGGGVSTVFALPSWQQGLAAQQTGGDSVPLARRGVPDVSADADPLTGYVVRVDGESGVVGGTSAAAPLWAALIARINAIKGRPAGYLHARLYQNPGAFNDIKQGNNGAFAAAPGWDACTGLGSPKGDAIANLF

SEQ ID NO: 11 - DNA, 1583 bp, synthetic construct
  misc_feature 1..1583: note = Description of Artificial Sequence: Synthetic polynucleotide
  source 1..1583: mol_type = other DNA
gaaataattttgtttaactttaagaaggagatatacatatgccgacctttctgctgcctggtagcgaacagacctgtccgcctggtgcacgttgtgttggtaaagcagatccgagcgcacgttttgaagttaccctggttgttcgtcagcctgcacaggatgcatttgcacgtcatctggaagcactgcatgatgttacccgtcgtcctccggcactgacccgtgaagcctatgcagcacagtatagcgcagcagcagatgattttgcagcagttgaacagtttgcagcaagcgaaggtctgcaggttgtgcgtcgtgatgcagcccagcgtaccattgttctgagcggcaccgttgcacagtttaatcatgcatttgaaatcgatctgcagaagattgaacacgagggtaaaagctatcgtggtcgtgttggtccggttcatctgccgcagcatctgaaaaccgttgttgatgcagttctgggtttagaagatctgccgctggcacgtacccattttcgtctgcagcctgcagcacgtagcgcagccggttttacaccgctggaactggcaagcatttatcagtttccggcaggcgcaggtaaaggtcaggccattgcactgattgaattaggtggtggtgttaaaaccagcgatctgaccacctattttagccagctgggtgttacccctccgcaggttaccgcagttagcgttgatcaggcaaccaatagtccgaccggtgatccgaatggtccggatggtgaagtgacactggatgttgaaattaccggtgcaattgcccctgaagcacatattgttctgtattttgcaccgaataccgaagccggtttctttaatgcagtttcagcagcagttcatgataccacacatcgtccgaccgttattagcattagctggggtggtccggaagcagcatggacccgtcagagcctggatgcctttgatcgtgcactgcaggcagccgcagcaatgggtgtgaccgtttgtgcagccagcggtgatagcggtagcagcggtagtcctggtaatggttcaccgcaggttgattttccggcaagcagtccgcatgttctggcatgtggtggcacccgtctgcatgcaagcgcaaatcgccgtgatgccgaaagcgtttggaatgatggtgcaggcggtggtgcaagtggtggtggcgttagcgcagcgtttgcactgccgagctggcaagagggcctgcaggttacagccgcagatggcaccagccaggcgctgacccagcgtggtgttccggatgttgccggtgatgcaagtccggcaagtggttatgatgttgttgtggatgcacaggccaccattgttggtggtacaagcgcagttgcaccgctgtgggcaggtctgattgcacgtctgaatgccagcctgggtaaaccgctgggttatctgaatccgattctgtatcagcatccgggtgttctgaatgatatcacccagggcgataatggtgaatttagtgcagcacctggttgggatgcatgtaccggtctgggtagcccgaatggccagaaaattgcgggtgttgcatga

SEQ ID NO: 12 - protein, 514 aa, Pandoraea thiooxydans
MPTFLLPGSEQTCPPGARCVGKADPSARFEVTLVVRQPAQDAFARHLEALHDVTRRPPALTREAYAAQYSAAADDFAAVEQFAASEGLQVVRRDAAQRTIVLSGTVAQFNHAFEIDLQKIEHEGKSYRGRVGPVHLPQHLKTVVDAVLGLEDLPLARTHFRLQPAARSAAGFTPLELASIYQFPAGAGKGQAIALIELGGGVKTSDLTTYFSQLGVTPPQVTAVSVDQATNSPTGDPNGPDGEVTLDVEITGAIAPEAHIVLYFAPNTEAGFFNAVSAAVHDTTHRPTVISISWGGPEAAWTRQSLDAFDRALQAAAAMGVTVCAASGDSGSSGSPGNGSPQVDFPASSPHVLACGGTRLHASANRRDAESVWNDGAGGGASGGGVSAAFALPSWQEGLQVTAADGTSQALTQRGVPDVAGDASPASGYDVVVDAQATIVGGTSAVAPLWAGLIARLNASLGKPLGYLNPILYQHPGVLNDITQGDNGEFSAAPGWDACTGLGSPNGQKIAGVA

SEQ ID NO: 13 - DNA, 1700 bp, synthetic construct
  misc_feature 1..1700: note = Description of Artificial Sequence: Synthetic polynucleotide
  source 1..1700: mol_type = other DNA
gaaataattttgtttaactttaagaaggagatatacatatgcgccatcgttttggtctgagcattctgtttctggttctggtgagcagcgcagttgcacaggttattgttccgcctaccagcgttcgtcgtccgggtgaacgtccgggtacagcacataccaattatcgtatctatattggtccgtggcgttttccgagcgttgatagcccgtttccggaactggcagcagcacatggtccggcagcaggtcagaccattccgggttatcatccggcagatattcgtgcagcatataatgttcctccgaatctgggcacccaggccattgcaattgttgatgcatttgatctgccgaccagcctgaatgattttaacttttttagcgcacagtttggcctgccgaccgaaccgagcggtgttgcaaccgcaagcaccaatcgtgtttttcaggttgtttatgcaagcggcaccaaaccggcaaccaatgcagattggggtggtgaaattgcactggatattgaatgggcacatgcaatggcaccgaatgcaaaaatctatctgattgaagcagatagcgatagcctgctggatctgctggcagccgttcgtgttgcagcaacccagctgagcaatgttcgtcagattagcatgagctttggtgccaatgaatttaccaatgaaagcgcaagcgatagcacctttctgggtacaaataaagttttttttgccagcagcggtgatgcaagcaatctggttagctatccggcagcgagcccgaatgttgttggtgttggtggcacccgtctggcactgagtaatggtagcgttgttagcgaaaccgcatggtcaagtgccggtggtggtccgagcagccgtgaaccgcgtccgacctatcagaatagcgttagcggtgtggttggtagcgcacgtggtacaccggatattgcagcaattgcagatccggaaaccggtgttgccgtttatgatagcaccccgattccaggtacaggtgttggttggtttgttgttggcggtacaagcctggcatgtccggtttgtgcaggtattaccaatgcacgtggttattttaccgccagcagctttagcgaactgacccgtctgtatggtctggcaggcaccagcttttttcgtgacattaccagcggcacctcaggtcagtttagtgcacgtgttggttatgattttgttaccggtctgggtagtctgctgggtatttttggtccgtttgcaaccagtccgagtagcctgagcgttgtgagcggcaccgcagttgccggtgttccgagcaatatggttgccaaagatggtcatgattatgttgttcgtagcgcaagtccggcaggcggtggtcaggttgccaccgttcagggcacctttgcaagccatccgcctgcaaaagcagttcagtttggtgcaagcgttaccgttaccgcaatgcgtaccagcggtacaaccacactgaaactgtttaatcaggcaaccagcgcatttgaaagcgttgcaaatctgaccctgggcaccaccaataccaccgtgaccgttccgattccgaatgcaccgaaatactttgcaagtgatggtacgaccaaatttcagctgaccaccacaggtcctggtacaacacagattcgctttggtgttgatcaggttctgctgaccctgacaccgacaggctga

SEQ ID NO: 14 - protein, 553 aa, Paraburkholderia sacchari
MRHRFGLSILFLVLVSSAVAQVIVPPTSVRRPGERPGTAHTNYRIYIGPWRFPSVDSPFPELAAAHGPAAGQTIPGYHPADIRAAYNVPPNLGTQAIAIVDAFDLPTSLNDFNFFSAQFGLPTEPSGVATASTNRVFQVVYASGTKPATNADWGGEIALDIEWAHAMAPNAKIYLIEADSDSLLDLLAAVRVAATQLSNVRQISMSFGANEFTNESASDSTFLGTNKVFFASSGDASNLVSYPAASPNVVGVGGTRLALSNGSVVSETAWSSAGGGPSSREPRPTYQNSVSGVVGSARGTPDIAAIADPETGVAVYDSTPIPGTGVGWFVVGGTSLACPVCAGITNARGYFTASSFSELTRLYGLAGTSFFRDITSGTSGQFSARVGYDFVTGLGSLLGIFGPFATSPSSLSVVSGTAVAGVPSNMVAKDGHDYVVRSASPAGGGQVATVQGTFASHPPAKAVQFGASVTVTAMRTSGTTTLKLFNQATSAFESVANLTLGTTNTTVTVPIPNAPKYFASDGTTKFQLTTTGPGTTQIRFGVDQVLLTLTPTG

SEQ ID NO: 15 - DNA, 1697 bp, synthetic construct
  misc_feature 1..1697: note = Description of Artificial Sequence: Synthetic polynucleotide
  source 1..1697: mol_type = other DNA
gaaataattttgtttaactttaagaaggagatatacatatgagcgatatggaaaaaccgtggaaagaagaagaaaaacgcgaagttctggcaggtcatgcacgtcgtcaggcaccgcaggcagttgataaaggtccggttaccggtgatcagcgtattagcgttaccgttgttctgcgtcgtcagcgtggtgatgaactggaagcacatgttgaacgtcaggcagcactggcaccgcatgcacgtgttcatctggaacgtgaagcatttgcagcaagccatggtgcaagcctggatgattttgcagaaattcgtaaatttgccgaagcgcatggtctgaccctggatcgtgcccatgttgcagcaggtacagcagttctgagcggtccggttgatgcagttaatcaggcatttggtgttgaactgcgtcattttgatcatcctgatggtagctatcgtagctatgttggtgatgttcgtgttccggcaagcattgcaccgctgattgaagcagttttaggtctggatacccgtccggttgcacgtccgcattttcgtctgcgtcgccgtgcagaaggtgaatttgaagcacgtagccagagcgcagcaccgaccgcatatacaccgctggatgttgcacaggcatatcagtttccggaaggcctggatggtcagggtcagtgtattgcaattattgaattaggtggtggctatgatgaaaccagcctggcacagtattttgccagcctgggtgttagcgctccgcaggttgttagcgttagcgtggatggtgcaaccaatcagccgacaggtgatccgaatggtccggatggtgaagttgaactggatattgaagttgccggtgcgctggcaccgggtgcaaaaattgcagtttattttgcaccgaataccgatgccggttttctgaatgcaattaccaccgcagttcatgatccgacacataaaccgagcattgtgagcattagctggggtggtccggaagatagctgggcaccagccagcattgcagccatgaatcgtgcatttctggatgcagccgcactgggtgtgaccgtgctggcagcagccggtgatagcggtagcaccgatggtgaacaggatggtctgtatcatgttgattttccggcagcgagcccgtatgttctggcatgtggtggcacccgtctggtggcaagcgcaggtcgtattgaacgtgaaaccgtttggaatgatggtcctgatggcggttcaaccggtggtggtgttagccgtatttttccgctgccgagctggcaagaacgtgcaaatgttccgcctagcgcaaatcctggtgcaggtagcggtcgtggtgttccggatgttgccggtaatgcagatccggcaaccggttatgaagttgttattgatggtgaaaccaccgtgattggtggtacaagcgcagtggcaccgctgtttgcagccctggttgcccgtattaatcagaaactgggtaaaccggttggttatctgaatccgacactgtatcagctgcctccggaagtttttcatgatattaccgaaggcaacaacgatattgccaatcgtgcacgtatttatcaggcaggtcctggttgggatccgtgtaccggtctgggtagcccgattggtattcgtctgctgcaggcactgctgccgagtgcaagccaggcacagccgtga

SEQ ID NO: 16 - protein, 552 aa, Bacillus sp.
MSDMEKPWKEEEKREVLAGHARRQAPQAVDKGPVTGDQRISVTVVLRRQRGDELEAHVERQAALAPHARVHLEREAFAASHGASLDDFAEIRKFAEAHGLTLDRAHVAAGTAVLSGPVDAVNQAFGVELRHFDHPDGSYRSYVGDVRVPASIAPLIEAVLGLDTRPVARPHFRLRRRAEGEFEARSQSAAPTAYTPLDVAQAYQFPEGLDGQGQCIAIIELGGGYDETSLAQYFASLGVSAPQVVSVSVDGATNQPTGDPNGPDGEVELDIEVAGALAPGAKIAVYFAPNTDAGFLNAITTAVHDPTHKPSIVSISWGGPEDSWAPASIAAMNRAFLDAAALGVTVLAAAGDSGSTDGEQDGLYHVDFPAASPYVLACGGTRLVASAGRIERETVWNDGPDGGSTGGGVSRIFPLPSWQERANVPPSANPGAGSGRGVPDVAGNADPATGYEVVIDGETTVIGGTSAVAPLFAALVARINQKLGKPVGYLNPTLYQLPPEVFHDITEGNNDIANRARIYQAGPGWDPCTGLGSPIGIRLLQALLPSASQAQP

SEQ ID NO: 17 - DNA, 1805 bp, synthetic construct
  misc_feature 1..1805: note = Description of Artificial Sequence: Synthetic polynucleotide
  source 1..1805: mol_type = other DNA
gaaataattttgtttaactttaagaaggagatatacatatgatgaaaagcagcgcagcaaaacagaccgttctgtgtctgaatcgttatgcagttgttgcactgccgctggcaattgcaagctttgcagcatttggtgcaagtccggcaagcaccctgtgggcaccgaccgataccaaagcatttgttacaccggcacaggttgaagcacgtagcgcagcaccgctgctggaactggcagccggtgaaaccgcacatattgttgttagcctgaaactgcgtgatgaagcacagctgaaacagctggcacaggcagttaatcagcctggtaatgcacagtttggcaaatttctgaaacgtcgtcagtttctgagccagtttgcaccgacagaagcacaggttcaggccgttgttgcccatctgcgtaaaaatggttttgtgaacattcatgttgtgccgaatcgtctgctgattagcgcagatggtagtgccggtgcagttaaagcagcatttaatacaccgctggttcgttatcagctgaatggtaaagcaggttatgcaaataccgcaccagcgcaggttccgcaggatctgggtgaaattgttggtagcgttctgggtctgcagaatgttacccgtgcacatccgatgctgaaagttggtgaacgtagtgcagcaaaaaccctggcagcaggcaccgcaaaaggtcataatccgaccgaatttccgaccatttatgatgccagcagcgctccgaccgcagcaaataccaccgtgggtattattaccattggtggtgttagtcagaccctgcaagatctgcagcagtttaccagcgcaaatggtctggcaagcgttaatacccagacaattcagaccggtagcagcaatggtgattattcagatgatcagcaaggtcaaggtgaatgggatttagatagccagagcattgttggttcagccggtggtgcagttcagcaactgctgttttatatggcagatcagagcgccagcggtaatacaggtctgacccaggcctttaatcaggcggttagcgataatgttgccaaagttattaatgtgagcttaggttggtgtgaagcagatgcaaatgcagatggcaccctgcaggcagaagatcgtatttttgcaaccgcagcagcccagggccagacctttagcgttagcagtggtgatgaaggtgtttatgaatgcaataatcgtggttatccggatggtagcacctatagcgtgagctggcctgcaagcagcccgaatgttattgccgttggtggtacaaccctgtataccaccagtgcgggtgcatatagcaatgaaaccgtttggaatgaaggtctggatagcaatggcaaactgtgggcaaccggtggtggttatagcgtgtatgaaagcaaaccgagctggcagagcgttgttagcggtacaccgggtcgccgtctgctgccggatattagctttgatgcagcacaaggtacaggtgcactgatttataactatggtcagctgcagcagattggtggcaccagcctggcaagcccgatttttgttggtttatgggcacgtctgcagagcgcaaatagcaatagcctgggttttccggcagccagcttttatagcgcaattagcagcaccccgagcctggttcatgatgttaaatcaggtaataatggctatggtggctacggttataatgccggtacaggttgggattatccgaccggttggggtagcctggatattgcaaaactgagcgcatatattcgtagcaacggttttggtcattga

SEQ ID NO: 18 - protein, 588 aa, Pseudomonas sp.
MMKSSAAKQTVLCLNRYAVVALPLAIASFAAFGASPASTLWAPTDTKAFVTPAQVEARSAAPLLELAAGETAHIVVSLKLRDEAQLKQLAQAVNQPGNAQFGKFLKRRQFLSQFAPTEAQVQAVVAHLRKNGFVNIHVVPNRLLISADGSAGAVKAAFNTPLVRYQLNGKAGYANTAPAQVPQDLGEIVGSVLGLQNVTRAHPMLKVGERSAAKTLAAGTAKGHNPTEFPTIYDASSAPTAANTTVGIITIGGVSQTLQDLQQFTSANGLASVNTQTIQTGSSNGDYSDDQQGQGEWDLDSQSIVGSAGGAVQQLLFYMADQSASGNTGLTQAFNQAVSDNVAKVINVSLGWCEADANADGTLQAEDRIFATAAAQGQTFSVSSGDEGVYECNNRGYPDGSTYSVSWPASSPNVIAVGGTTLYTTSAGAYSNETVWNEGLDSNGKLWATGGGYSVYESKPSWQSVVSGTPGRRLLPDISFDAAQGTGALIYNYGQLQQIGGTSLASPIFVGLWARLQSANSNSLGFPAASFYSAISSTPSLVHDVKSGNNGYGGYGYNAGTGWDYPTGWGSLDIAKLSAYIRSNGFGH

SEQ ID NO: 19 - DNA, 1673 bp, synthetic construct
  misc_feature 1..1673: note = Description of Artificial Sequence: Synthetic polynucleotide
  source 1..1673: mol_type = other DNA
gaaataattttgtttaactttaagaaggagatatacatatggccaacggtaaaagcaccagtccggcaagccagtgggttccgctgcctggtagcaatcgtcagctgctgccgcagagcgttccgattggtccggcagatctgaaagcaaccgttgcactgaccgttaaagttcgtagccgtggtaaactggcagaactggatgatgcagttaaaaaagaaagcgcaaaaccgctgaaagaacgcacctatattagccgtgaagaactggcacagcgttatggtgcagatgcagatgatctggataaagttgaactgtatgccaacaaacatcatctgcgtgttgcagatcgtgatgaagcaacccgtcgtgttgttctgaaaggcaccctggaagatgcactgagcgcatttcatgcagatgttcacatgtatcagcatgcaagcggtccgtatcgtggtcgtcgtggtgaaattctggttcctgcagaactgaaagatgttgtgaccggtatttttggctttgatacccatccgaaacatcgtgcaccgcgtcgtctgatgggcaccagcagcggcaccgcaaccaatctgggtgaatttgcaagcgaatttgcgacccgttatcagtttccgaccagcagcagcagtaccaaactggatggcaccggtcagtgtattgcactgattgaattaggtggtggctatagcaataacgatctgaaaatcttttttagcgaagccggtgttccgatgccgaaagttgttgcagttagcattgatcatggtgcaaatcatccgacaccgcaaggtctggcagatggtgaagttatgctggatattgaagttgccggtgttgttgcaccgggtgccaaactggccgtttattttgcaccgaatagcgatagcggttttcaggatgcaattcgtgcagcagttcatgatggtgcacgtaaaccgagcgttgttagcattagctggggtgaacctgatgattttctgaccgcacagagcgtgcagagctatcatgaaatctttaccgaagcagcagccctgggtgttaccgtttgtgcagcaagcggtgatcatggcgttgccgatctggatgcactgcattgggataaacgtattcatgttaatcatccgtcaagcgatccgctggttctgtgttgtggtggtacacagattgataaaaatgttgatgtggtgtggaatgatggcaccccgtttgatccgcaggtttttggtggtggcggttgggccagcggtggtggtattagtccggtgtttggtgttccggattatcagaaaggtctgccgatgccgtcaagcctgagcaccagccagcctggtcgtggttgtccggatattgcaatgaccgcagataactatcgtacccgtgttcatggtgttgatggtccgagcggtggcaccagcgcagttacaccgctgatggcatgtctggttgcacgtctgaatcaggcatttgaaaaaaatctgggttttgtgaatccgctgctgtatgcaaatgcacaggcatttaccgatattacccagggcaccaatggtattaatcagaccattgaaggttatccggcaggtaaaggttgggatgcatgtaccggtctgggtgcaccgattggcaccgttctgctgcaggcactgggtaaatga

SEQ ID NO: 20 - protein, 544 aa, Variovorax sp.
MANGKSTSPASQWVPLPGSNRQLLPQSVPIGPADLKATVALTVKVRSRGKLAELDDAVKKESAKPLKERTYISREELAQRYGADADDLDKVELYANKHHLRVADRDEATRRVVLKGTLEDALSAFHADVHMYQHASGPYRGRRGEILVPAELKDVVTGIFGFDTHPKHRAPRRLMGTSSGTATNLGEFASEFATRYQFPTSSSSTKLDGTGQCIALIELGGGYSNNDLKIFFSEAGVPMPKVVAVSIDHGANHPTPQGLADGEVMLDIEVAGVVAPGAKLAVYFAPNSDSGFQDAIRAAVHDGARKPSVVSISWGEPDDFLTAQSVQSYHEIFTEAAALGVTVCAASGDHGVADLDALHWDKRIHVNHPSSDPLVLCCGGTQIDKNVDVVWNDGTPFDPQVFGGGGWASGGGISPVFGVPDYQKGLPMPSSLSTSQPGRGCPDIAMTADNYRTRVHGVDGPSGGTSAVTPLMACLVARLNQAFEKNLGFVNPLLYANAQAFTDITQGTNGINQTIEGYPAGKGWDACTGLGAPIGTVLLQALGK

SEQ ID NO: 21 - DNA, 1581 bp, synthetic construct
  misc_feature 1..1581: note = Description of Artificial Sequence: Synthetic polynucleotide
  source 1..1581: mol_type = other DNA
gaaataattttgtttaactttaagaaggagatatacatatgaaaaccagcaacaaagttgcactggcaggtagctacaaaaaagcacatagcggtgaaaccaccgccaaaattaaccgtaatacctttattgaagtgaccctgcgtattcgtcgcaaaaaaagcattgaaagcctgctgaatgcaggtaaacgtgttgatcatgccgattacgaaaaagaatttggtgcaagccagaaagatgcagatcaggttgaagcatttgcacgtcagtataaactgagcaccgttgaagttagcctgagccgtcgtagcgttattctgcgtggtagcattgcaaatatggaagcagcatttgatgtgaatctgagcaaagcagttgatagccatggtgatgatattcgtgttcgtaaaggcgatatctatattccggaagcactgaaagatgttgtggaaggtgtttttggtctggataatcgtaaagcagcacgtccgctgtttaaactgctgaaaaaagcagatggtattagtccgcaggcaagcgttagcagcagctttaccccgaatcagctggcaggcatttatggttttccggcaggttttaatggtaaaggtcagaccattgccattattgaattaggtggtggttatcgtaccaccgatctgaccaattatttcaaaaaactgggcatcaaaaaaccgtccattaaagccattctggtggacaaaggtaaaaacaatccgagcaatgcaaatagcgcagatggtgaagttatgctggatattgaagttgccggtgcagttgcaagcggtgcaaaaattgttgtgtattttagcccgaataccgacaaaggttttctggatgcaattaccaaagccgttcatgataccacacataaaccgagcgttgttagcattagctggggtggtggtgaagcagtttggacccagcagagcctgaatagttttaatgaagcctttaaagcagccgcagttctgggtgttaccgtttgtgcagcagccggtgataatggtagcagtgatggcctgaccgataatagcgttcatgttgattttccagcaagcagcccgtatgttctggcatgtggtggtacaaccctgaaagtgaaaaacaatgttattaccagcgaaaccgtttggcatgatagcaatgatagcgcaaccggtggtggcgttagcaatgtttttccgctgccggattatcagaaaaatgccggtgttccggcagcaattggcaccaactttattggtcgtggtgtgccggatgttgcaggtaatgcagatccgaatacaggttataatgttctggttgatggtcagcagctggttattggtggcaccagcgcagtggcaccgctgtttgcaggtctgattgcatgtctgaatcagaaaagcggtaaatggtcaggttttatcaatccgacactgtatgcagcaaatccgagcgtttgtcgtgatattaccgttggtaataatcgtaccgccaccggtaatgccggttatgatgcacgtgttggttgggatccgtgtaccggtctgggtgtgtttagcaaactgctga

SEQ ID NO: 22 - protein, 513 aa, Mucilaginibacter sp.
MKTSNKVALAGSYKKAHSGETTAKINRNTFIEVTLRIRRKKSIESLLNAGKRVDHADYEKEFGASQKDADQVEAFARQYKLSTVEVSLSRRSVILRGSIANMEAAFDVNLSKAVDSHGDDIRVRKGDIYIPEALKDVVEGVFGLDNRKAARPLFKLLKKADGISPQASVSSSFTPNQLAGIYGFPAGFNGKGQTIAIIELGGGYRTTDLTNYFKKLGIKKPSIKAILVDKGKNNPSNANSADGEVMLDIEVAGAVASGAKIVVYFSPNTDKGFLDAITKAVHDTTHKPSVVSISWGGGEAVWTQQSLNSFNEAFKAAAVLGVTVCAAAGDNGSSDGLTDNSVHVDFPASSPYVLACGGTTLKVKNNVITSETVWHDSNDSATGGGVSNVFPLPDYQKNAGVPAAIGTNFIGRGVPDVAGNADPNTGYNVLVDGQQLVIGGTSAVAPLFAGLIACLNQKSGKWSGFINPTLYAANPSVCRDITVGNNRTATGNAGYDARVGWDPCTGLGVFSKL

SEQ ID NO: 23 - DNA, 1724 bp, synthetic construct
  misc_feature 1..1724: note = Description of Artificial Sequence: Synthetic polynucleotide
  source 1..1724: mol_type = other DNA
gaaataattttgtttaactttaagaaggagatatacatatggcaccgaaaaccagcgttccgcattttaccacacagagccgtaccgttctgagcggtagcgaaaaagcaccggttgccgaagcacgtggtgcaaaaccggcaccgctggcagcacgtattaccgttagcgttattgttcgtcgtaaaacaccgctgaaagcagcccatattaccggtgaacagcgtctgacccgtgcacagtttaatgcaagccatgcagcagatccggcagcagttaaactggttcagggttttgccaaagaatttggtctgaccgttgatccgggtactccggcaccgggtcgtcgtaccatgaaactgaccggtacagtggcaaatatgcagcgtgcatttggtgttagcctggcacataaaaccatggatggtgttacctatcgtgttcgtgaaggtagcattaatctgcctgcagaactgcagggttatgttgttgcagttttaggtctggataatcgtccgcaggcagaaccgcattttcgtattctgggtgaacagggtgcagttgcagcacaggcagcacaaggtcagggctttgcaggtccgcatgccggtggtagcaccagctatacaccggttcaggttggtgaactgtatcagtttccgcgtggtagcagcgcaagcaatcagaccattggtattattgaattaggtggtggttttcgccagaccgatattgcagcatactttaaaaccctgggtcagaaaccgcctcaggttattgcagttccgattggtaatggtaaaaacaatccgaccaatagcaatagcgcagatggtgaagttatgctggatattgaagttgccggtgccgttgcaccgggtgcacgtattgttgtttattttgcaccgaataccgatcagggtttcgttgatgcaattgcccatgcaattcatgataccacctataaaccgagcgttattagcattagctggggtagcgcagaagttaattggaccgttcaggcaatggcagcactggatgcagcatgtcagagcgcagcagccctgggtattacaattaccgcagcaagcggtgataatggtagcagtgatgcagttgccgatggtgaaaatcatgttgattttccggcaagcagtccgcatgttctggcatgtggtggcaccaatctgcaaggtagcggtagtaccattagtgcagaaaccgtttggaatgcacagccgcaaggtggtgcgaccggtggtggtgtgagcaacatttttccgctgccgacctggcaggcaagcagcaaagttccgaaaccgacacatccgagcggtggtcgtggtgttccggatgttgcgggtgatgccgatccggcaagtggttatgtggttcgtgttgatggtcagacctttgttattggtggtacaagcgcagttgcaccgctgtgggcaggcctgattgcagttgcgaatcagcagaatggtaaatcagcaggttttattcagcctgcaatttatgcaggtcagggtaaaccggcatttcgtgataccgtgcagggtagcaatggtagctttgcagcaggcgcaggttgggatgcatgcaccggtctgggtagcccgattgcactgcagctgattaacgcaatcaaaccggcaagctcaaaaagcaaaagcaaagcgattgcagcaaaacgcaaaaccattatccgtaccaaaaaatga

SEQ ID NO: 24 - protein, 561 aa, Paraburkholderia sacchari
MAPKTSVPHFTTQSRTVLSGSEKAPVAEARGAKPAPLAARITVSVIVRRKTPLKAAHITGEQRLTRAQFNASHAADPAAVKLVQGFAKEFGLTVDPGTPAPGRRTMKLTGTVANMQRAFGVSLAHKTMDGVTYRVREGSINLPAELQGYVVAVLGLDNRPQAEPHFRILGEQGAVAAQAAQGQGFAGPHAGGSTSYTPVQVGELYQFPRGSSASNQTIGIIELGGGFRQTDIAAYFKTLGQKPPQVIAVPIGNGKNNPTNSNSADGEVMLDIEVAGAVAPGARIVVYFAPNTDQGFVDAIAHAIHDTTYKPSVISISWGSAEVNWTVQAMAALDAACQSAAALGITITAASGDNGSSDAVADGENHVDFPASSPHVLACGGTNLQGSGSTISAETVWNAQPQGGATGGGVSNIFPLPTWQASSKVPKPTHPSGGRGVPDVAGDADPASGYVVRVDGQTFVIGGTSAVAPLWAGLIAVANQQNGKSAGFIQPAIYAGQGKPAFRDTVQGSNGSFAAGAGWDACTGLGSPIALQLINAIKPASSKSKSKAIAAKRKTIIRTKK

SEQ ID NO: 25 - protein, 523 aa, synthetic construct
  REGION 1..523: note = Description of Artificial Sequence: Synthetic polypeptide
MSEPVPAAARRTIPGSERPPVDTAAAARQAVPADTRVEATVVLRRRAELPDGPGLLTPAELAERHGADPADVELVTRTLTGLGVEVTAVDAASRRLRVAGPAGVLAEAFGTSLAQVSTPDPSGAQVTHRYRAGALSVPAELDGVVTAVLGLDDRPQARARFRVATAAAASAGYTPIELGRVYSFPEGSDGSGQTIAIIELGGGFAQSELDTYFAGLGISGPTVTAVGVDGGSNVAGRDPQGADGEVLLDIEVAGALAPGADVVVYFAPNTDAGFLDAVAQAAHATPTPAAISISWGGSEDTWTGQARTAFDAALADAAALGVTTTVAAGDDGSTDRATDGKSHVDFPASSPHALACGGTHLDANATTGAVTSEVVWNNGAGKGATGGGVSTVFAQPSWQASAGVPDGPGGKPGRGVPDVSAVADPQTGYRIRVDGQDLVIGGTSAVAPLWAALVARLVQAGRAKLGLLQPKLYAAPTAFRDITEGDNGAYRAGPGWDACTGLGVPVGTALASALSLEHHHHHH

SEQ ID NO: 26 - protein, 539 aa, synthetic construct
  REGION 1..539: note = Description of Artificial Sequence: Synthetic polypeptide
MADDSSPTTAADRPTLPGSARRPVAAAQAAGPLDDAAPLEVTLVLRRRTALPAGTGRPAPMGRAEFAETHGADPADAETVTAALTAEGLRITAVDLPSRRVQVAGDVATFSRVFGVSLSRVESPDPVADRLVPHRQRSGDLAVPAPLAGVVTAVLGLDDRPQARALFRPAAAVDTTFTPLELGRVYRFPSGTDGRGQRLAILELGGGYTQADLDAYWTTIGLADPPTVTAVGVDGAANAPEGDPNGADGEVLLDIEVAGALAPGADLVVYFAPNTDRGFLDALSTAVHADPTPTAVSISWGQNEDEWTAQARTAMDEALADAAALGVTVCAAAGDDGSTDNAPDGQAHVDFPASSPHALACGGTTLRADPDTGEVSSETVWFHGTGQGGTGGGVSAVFAVPDWQDGVRVPGDADTGRHGRGVPDVSADADPSTGYQVRVDGTDAVFGGTSAVSPLWSALTCRLAEALGQRPGLLQPLIYAGLSAGEVAAGFRDVTSGSNGAYDAGPGWDPCTGLGVPDGEALLVRLRTALGLEHHHHHH

SEQ ID NO: 27 - protein, 532 aa, synthetic construct
  REGION 1..532: note = Description of Artificial Sequence: Synthetic polypeptide
MDYQILRGSERSPLPGCTDTGKFPAAHRLRVLLALRQPELDAAAARLLDTAGDELPAPLSRDAFATRFAAAADDLRAVEAFATQHGLSMEQTLAHAGVAILEGSVQQFDRAFQVDLRDYRKDDLRYRGRTGAVSIPTALHGVVSAVLGLDDRPQAHTLPQAQDAPAPAGAAAPIARYTPPQLAELYGFPEHDGAGQCIGIIALGGGYERAQLAAYFTELGLPMPQIVDVLLAGARNQPGGQGRKADIEVQMDVQIAGAIAPGAKLVVYFAPNTDNGFLEAIVSAIHDRAHAPDVIAISWGFTETLWTAQSRAAYNRALQAAALMGITVCIASGDDGASDGQPGLNVCFPASSPFVLACGGTRLQVDVQAQHEQAWSGTGGGQSRVFARPRWQQALTLHGTQQTAQPLSMRGVPDVAANADAETGYYVHIDGRPAVMGGTSAAAPVWAALLARVYGLNGGRRVFLPPRLYAVADVCRDIVDGGNGGFVASPGWDACTGLGVPDGGRIAAALGAGPGAKPAITPTGLEHHHHHH

SEQ ID NO: 28 - protein, 558 aa, synthetic construct
  REGION 1..558: note = Description of Artificial Sequence: Synthetic polypeptide
MTRHPVSDSGASNEHPVPAGAQCMGACDPAEHFNVVVIVRRQSERAFRELVERIATGAPGAQPISREQYEQRFSADAADVARVEAFAKTHGLVVVKADRDTRRVVLSGTVQQYNAAFGVDLQRFEHQVGKLKQHFRQPTGPVHLPEDLHEVITAVVGLDSRAKVQPHFRIDSQTPATPPEKASQPGDGVVHAPIRAARAVSRSFTPLQLAELYDFPPGDGKGQCIALIEMGGGYAQSDLDAYFSALGVTRPRVEAVSVDQATNAPSGDPNGPDAEVTLDVEIAGALAPGALIAVYFAPNSEAGFVDAVSAALHDSQRKAAIISISWGAPESIWSQQTLGALNDALQTAVALGVTVCCASGDSGSSDGVTDGADHVDFPASSPYALGCGGTQLTAANGRITRETVWGSGANGATGGGVSATFAVPAWQKGLKVSRGSGAARALALARRGVPDVAADADPATGYEVHIGGMDTVVGGTSAVAPLWAALVARINAGSGKAAGFINAKLYARPGAFNDITSGSNGDYAARPGWDACTGLGTPVGTRVAAAIGSALEHHHHHH

SEQ ID NO: 29 - protein, 540 aa, synthetic construct
  REGION 1..540: note = Description of Artificial Sequence: Synthetic polypeptide
MVRHPLRGSERTIPEDARILGDAHPAEQIRALVQLRRPNEAELDVRLSGFVHAHAAGTPSPTPLTREEWAAQFGAATDDIDAVRTFAREHGLQVAEVNVAAATVMLEGSVEQFCRAFDTHLHRVAHGGSEYRGRSGPLRLPESLQDVVVAVLGLDSRPQAAPHFRFVPLPTGSVEPGGIRPARAAPTASYTPVQLAQLYGFPQGDGAGQCIAFVELGGGYREDDLRAYFQEVGMPMPTVTAIPVGQGANRPTGDPSGPDGEVMLDLEVAGAAAPGATLAVYFTVNTDAGFVQAINAAIHDTKLRPSVVSISWGAPESAWTPQAMQAVNAALQSAATMGVTVCAASGDSGSSDGQPDRVDHVDFPASSPYALACGGTSVRASGNRIAEETVWNDGARGGAGGGGVSTVFALPSWQQGLAAQQTGGDSVPLARRGVPDVSADADPLTGYVVRVDGESGVVGGTSAAAPLWAALIARINAIKGRPAGYLHARLYQNPGAFNDIKQGNNGAFAAAPGWDACTGLGSPKGDAIANLFLEHHHHHH

SEQ ID NO: 30 - protein, 522 aa, synthetic construct
  REGION 1..522: note = Description of Artificial Sequence: Synthetic polypeptide
MPTFLLPGSEQTCPPGARCVGKADPSARFEVTLVVRQPAQDAFARHLEALHDVTRRPPALTREAYAAQYSAAADDFAAVEQFAASEGLQVVRRDAAQRTIVLSGTVAQFNHAFEIDLQKIEHEGKSYRGRVGPVHLPQHLKTVVDAVLGLEDLPLARTHFRLQPAARSAAGFTPLELASIYQFPAGAGKGQAIALIELGGGVKTSDLTTYFSQLGVTPPQVTAVSVDQATNSPTGDPNGPDGEVTLDVEITGAIAPEAHIVLYFAPNTEAGFFNAVSAAVHDTTHRPTVISISWGGPEAAWTRQSLDAFDRALQAAAAMGVTVCAASGDSGSSGSPGNGSPQVDFPASSPHVLACGGTRLHASANRRDAESVWNDGAGGGASGGGVSAAFALPSWQEGLQVTAADGTSQALTQRGVPDVAGDASPASGYDVVVDAQATIVGGTSAVAPLWAGLIARLNASLGKPLGYLNPILYQHPGVLNDITQGDNGEFSAAPGWDACTGLGSPNGQKIAGVALEHHHHHH

SEQ ID NO: 31 - protein, 561 aa, synthetic construct
  REGION 1..561: note = Description of Artificial Sequence: Synthetic polypeptide
MRHRFGLSILFLVLVSSAVAQVIVPPTSVRRPGERPGTAHTNYRIYIGPWRFPSVDSPFPELAAAHGPAAGQTIPGYHPADIRAAYNVPPNLGTQAIAIVDAFDLPTSLNDFNFFSAQFGLPTEPSGVATASTNRVFQVVYASGTKPATNADWGGEIALDIEWAHAMAPNAKIYLIEADSDSLLDLLAAVRVAATQLSNVRQISMSFGANEFTNESASDSTFLGTNKVFFASSGDASNLVSYPAASPNVVGVGGTRLALSNGSVVSETAWSSAGGGPSSREPRPTYQNSVSGVVGSARGTPDIAAIADPETGVAVYDSTPIPGTGVGWFVVGGTSLACPVCAGITNARGYFTASSFSELTRLYGLAGTSFFRDITSGTSGQFSARVGYDFVTGLGSLLGIFGPFATSPSSLSVVSGTAVAGVPSNMVAKDGHDYVVRSASPAGGGQVATVQGTFASHPPAKAVQFGASVTVTAMRTSGTTTLKLFNQATSAFESVANLTLGTTNTTVTVPIPNAPKYFASDGTTKFQLTTTGPGTTQIRFGVDQVLLTLTPTGLEHHHHHH

SEQ ID NO: 32 - protein, 560 aa, synthetic construct
  REGION 1..560: note = Description of Artificial Sequence: Synthetic polypeptide
MSDMEKPWKEEEKREVLAGHARRQAPQAVDKGPVTGDQRISVTVVLRRQRGDELEAHVERQAALAPHARVHLEREAFAASHGASLDDFAEIRKFAEAHGLTLDRAHVAAGTAVLSGPVDAVNQAFGVELRHFDHPDGSYRSYVGDVRVPASIAPLIEAVLGLDTRPVARPHFRLRRRAEGEFEARSQSAAPTAYTPLDVAQAYQFPEGLDGQGQCIAIIELGGGYDETSLAQYFASLGVSAPQVVSVSVDGATNQPTGDPNGPDGEVELDIEVAGALAPGAKIAVYFAPNTDAGFLNAITTAVHDPTHKPSIVSISWGGPEDSWAPASIAAMNRAFLDAAALGVTVLAAAGDSGSTDGEQDGLYHVDFPAASPYVLACGGTRLVASAGRIERETVWNDGPDGGSTGGGVSRIFPLPSWQERANVPPSANPGAGSGRGVPDVAGNADPATGYEVVIDGETTVIGGTSAVAPLFAALVARINQKLGKPVGYLNPTLYQLPPEVFHDITEGNNDIANRARIYQAGPGWDPCTGLGSPIGIRLLQALLPSASQAQPLEHHHHHH

SEQ ID NO: 33 - protein, 596 aa, synthetic construct
  REGION 1..596: note = Description of Artificial Sequence: Synthetic polypeptide
MMKSSAAKQTVLCLNRYAVVALPLAIASFAAFGASPASTLWAPTDTKAFVTPAQVEARSAAPLLELAAGETAHIVVSLKLRDEAQLKQLAQAVNQPGNAQFGKFLKRRQFLSQFAPTEAQVQAVVAHLRKNGFVNIHVVPNRLLISADGSAGAVKAAFNTPLVRYQLNGKAGYANTAPAQVPQDLGEIVGSVLGLQNVTRAHPMLKVGERSAAKTLAAGTAKGHNPTEFPTIYDASSAPTAANTTVGIITIGGVSQTLQDLQQFTSANGLASVNTQTIQTGSSNGDYSDDQQGQGEWDLDSQSIVGSAGGAVQQLLFYMADQSASGNTGLTQAFNQAVSDNVAKVINVSLGWCEADANADGTLQAEDRIFATAAAQGQTFSVSSGDEGVYECNNRGYPDGSTYSVSWPASSPNVIAVGGTTLYTTSAGAYSNETVWNEGLDSNGKLWATGGGYSVYESKPSWQSVVSGTPGRRLLPDISFDAAQGTGALIYNYGQLQQIGGTSLASPIFVGLWARLQSANSNSLGFPAASFYSAISSTPSLVHDVKSGNNGYGGYGYNAGTGWDYPTGWGSLDIAKLSAYIRSNGFGHLEHHHHHH

SEQ ID NO: 34 - protein, 552 aa, synthetic construct
  REGION 1..552: note = Description of Artificial Sequence: Synthetic polypeptide
MANGKSTSPASQWVPLPGSNRQLLPQSVPIGPADLKATVALTVKVRSRGKLAELDDAVKKESAKPLKERTYISREELAQRYGADADDLDKVELYANKHHLRVADRDEATRRVVLKGTLEDALSAFHADVHMYQHASGPYRGRRGEILVPAELKDVVTGIFGFDTHPKHRAPRRLMGTSSGTATNLGEFASEFATRYQFPTSSSSTKLDGTGQCIALIELGGGYSNNDLKIFFSEAGVPMPKVVAVSIDHGANHPTPQGLADGEVMLDIEVAGVVAPGAKLAVYFAPNSDSGFQDAIRAAVHDGARKPSVVSISWGEPDDFLTAQSVQSYHEIFTEAAALGVTVCAASGDHGVADLDALHWDKRIHVNHPSSDPLVLCCGGTQIDKNVDVVWNDGTPFDPQVFGGGGWASGGGISPVFGVPDYQKGLPMPSSLSTSQPGRGCPDIAMTADNYRTRVHGVDGPSGGTSAVTPLMACLVARLNQAFEKNLGFVNPLLYANAQAFTDITQGTNGINQTIEGYPAGKGWDACTGLGAPIGTVLLQALGKLEHHHHHH

SEQ ID NO: 35 - protein, 521 aa, synthetic construct
  REGION 1..521: note = Description of Artificial Sequence: Synthetic polypeptide
MKTSNKVALAGSYKKAHSGETTAKINRNTFIEVTLRIRRKKSIESLLNAGKRVDHADYEKEFGASQKDADQVEAFARQYKLSTVEVSLSRRSVILRGSIANMEAAFDVNLSKAVDSHGDDIRVRKGDIYIPEALKDVVEGVFGLDNRKAARPLFKLLKKADGISPQASVSSSFTPNQLAGIYGFPAGFNGKGQTIAIIELGGGYRTTDLTNYFKKLGIKKPSIKAILVDKGKNNPSNANSADGEVMLDIEVAGAVASGAKIVVYFSPNTDKGFLDAITKAVHDTTHKPSVVSISWGGGEAVWTQQSLNSFNEAFKAAAVLGVTVCAAAGDNGSSDGLTDNSVHVDFPASSPYVLACGGTTLKVKNNVITSETVWHDSNDSATGGGVSNVFPLPDYQKNAGVPAAIGTNFIGRGVPDVAGNADPNTGYNVLVDGQQLVIGGTSAVAPLFAGLIACLNQKSGKWSGFINPTLYAANPSVCRDITVGNNRTATGNAGYDARVGWDPCTGLGVFSKLLEHHHHHH

SEQ ID NO: 36 - protein, 569 aa, synthetic construct
  REGION 1..569: note = Description of Artificial Sequence: Synthetic polypeptide
MAPKTSVPHFTTQSRTVLSGSEKAPVAEARGAKPAPLAARITVSVIVRRKTPLKAAHITGEQRLTRAQFNASHAADPAAVKLVQGFAKEFGLTVDPGTPAPGRRTMKLTGTVANMQRAFGVSLAHKTMDGVTYRVREGSINLPAELQGYVVAVLGLDNRPQAEPHFRILGEQGAVAAQAAQGQGFAGPHAGGSTSYTPVQVGELYQFPRGSSASNQTIGIIELGGGFRQTDIAAYFKTLGQKPPQVIAVPIGNGKNNPTNSNSADGEVMLDIEVAGAVAPGARIVVYFAPNTDQGFVDAIAHAIHDTTYKPSVISISWGSAEVNWTVQAMAALDAACQSAAALGITITAASGDNGSSDAVADGENHVDFPASSPHVLACGGTNLQGSGSTISAETVWNAQPQGGATGGGVSNIFPLPTWQASSKVPKPTHPSGGRGVPDVAGDADPASGYVVRVDGQTFVIGGTSAVAPLWAGLIAVANQQNGKSAGFIQPAIYAGQGKPAFRDTVQGSNGSFAAGAGWDACTGLGSPIALQLINAIKPASSKSKSKAIAAKRKTIIRTKKLEHHHHHH

SEQ ID NO: 37 - protein, 8 aa, synthetic construct
  REGION 1..8: note = Description of Artificial Sequence: Synthetic peptide
LEHHHHHH

SEQ ID NO: 38 - protein, 16 aa, synthetic construct
  REGION 1..16: note = Description of Artificial Sequence: Synthetic peptide
EFSWGAAGDDDGGTSA

SEQ ID NO: 39 - protein, 16 aa, synthetic construct
  REGION 1..16: note = Description of Artificial Sequence: Synthetic peptide
EFSWGASGDDCGGTSA

SEQ ID NO: 40 - protein, 16 aa, synthetic construct
  REGION 1..16: note = Description of Artificial Sequence: Synthetic peptide
EFSWGASGDSDGGTSA

SEQ ID NO: 41 - protein, 16 aa, synthetic construct
  REGION 1..16: note = Description of Artificial Sequence: Synthetic peptide
ELSFGSSGDASGGTSL

SEQ ID NO: 42 - protein, 16 aa, synthetic construct
  REGION 1..16: note = Description of Artificial Sequence: Synthetic peptide
EFSWGAAGDSDGGTSA

SEQ ID NO: 43 - protein, 16 aa, synthetic construct
  REGION 1..16: note = Description of Artificial Sequence: Synthetic peptide
ELSLGSSGDESGGTSL

SEQ ID NO: 44 - protein, 16 aa, synthetic construct
  REGION 1..16: note = Description of Artificial Sequence: Synthetic peptide
EFSWGASGDHNGGTSA

SEQ ID NO: 45 - protein, 16 aa, synthetic construct
  REGION 1..16: note = Description of Artificial Sequence: Synthetic peptide
EFSWGAAGDNDGGTSA

SEQ ID NO: 46 - protein, 16 aa, synthetic construct
  REGION 1..16: note = Description of Artificial Sequence: Synthetic peptide
EFSWGASGDNDGGTSA